(19) United States
(12) Patent Application Publication
(10) Pub. No.: US 2013/0254945 A1
(43) Pub. Date: Sep. 26, 2013

(54) SOYBEAN APHID RESISTANCE GENE RAG2

(71) Applicants: Ki-Seung Kim, Urbana, IL (US); Curtis B. Hill, Champaign, IL (US); Glen L. Hartman, Urbana, IL (US); David L. Hyten, Johnston, IA (US); Matthew E. Hudson, Champaign, IL (US); Brian W. Diers, Urbana, IL (US); Tong Geon Lee, Urbana, IL (US)

(72) Inventors: Ki-Seung Kim, Urbana, IL (US); Curtis B. Hill, Champaign, IL (US); Glen L. Hartman, Urbana, IL (US); David L. Hyten, Johnston, IA (US); Matthew E. Hudson, Champaign, IL (US); Brian W. Diers, Urbana, IL (US); Tong Geon Lee, Urbana, IL (US)

(73) Assignees: United States of America as represented by the Secretary of Agriculture, Washington, DC (US); THE BOARD OF TRUSTEES OF THE UNIVERSITY OF ILLINOIS, Urbana, IL (US)

(21) Appl. No.: 13/605,576

(22) Filed: Sep. 6, 2012

Related U.S. Application Data
(63) Continuation-in-part of application No. PCT/US11/28726, filed on Mar. 16, 2011.
(60) Provisional application No. 61/314,982, filed on Mar. 17, 2010.

(51) Int. Cl. C12N 15/82

(52) U.S. Cl. CPC C12N 15/8286
USPC 800/302; 530/300; 536/23.6; 435/419; 435/69.1; 435/6.11; 536/24.3

(57) ABSTRACT

Provided herein are isolated nucleic acid molecules representing a genetically defined region of the genome of the aphid resistant soybean plant (Glycine max) cultivar PI 200538 that confers resistance to soybean aphid (Aphis glycines). Within the region is a gene encoding the aphid resistance protein Rag2. Also provided herein are methods for conferring aphid resistance on a plant or enhancing aphid resistance in a plant by transforming it to contain and express such nucleic acid sequences encoding Rag2 aphid resistance or introgressing DNA encoding the trait into the plant by plant breeding. Further provided are polymorphic markers useful for identifying plant germplasm containing aphid resistance, and methods for using such markers.

### CROSS-REFERENCE TO RELATED APPLICATIONS

This application claims priority to PCT Application No. PCT/US2011/028726, a continuation-in-part of U.S. Provisional Patent Application Ser. No. 61/314,982, filed Mar. 17, 2010, both of which are incorporated herein by reference to the extent not inconsistent herewith.

### STATEMENT OF GOVERNMENT SUPPORT

This invention was made with government support under contract number #AG2006-34488-16915 and 2010-34488-21109, awarded by the USDA CSREES. The government has certain rights in this invention.

### BACKGROUND

This disclosure relates to nucleic acid molecules, polypeptides and methods related to a gene which determines resistance to aphids in a plant. Although the soybean aphid (SA: Aphis glycines Matsumura) has a short history in the USA, the aphid has caused significant damage and has spread to most soybean growing states since its discovery in the country in 2000 (Voegtlin 2008). There is a need to identify new sources of SA resistance as diversity in SA was shown to occur in North America. Michel et al. (2009) showed the presence of diversity in SA based on simple sequence repeat (SSR) diversity and Kim et al. (2008) showed that at least two different SA biotypes exit in North America. These biotypes are biotype 1, which was collected in Illinois and is controlled by the SA-resistance genes Rag1 from Dowling and Rag in ‘Jackson’, and biotype 2, which originated from Ohio and can overcome these genes. Evaluations with both SA biotypes 1 and 2 show that resistance in plant introduction (PI) 200538 is not defeated by either SA biotype and thus this PI is a potential source of new, useful SA-resistance gene(s) (Kim et al. 2008). Recently, a new SA biotype was collected from the overwintering host glossy buckthorn (Frangula alnus) that can readily colonize plants with Rag2 (Hill et al. 2010). This has been named biotype 3 and it demonstrates the continued necessity to identity new SA-resistance genes and to stack the genes in soybean cultivars.

Several research groups have identified soybean genotypes with SA resistance and have mapped resistance genes onto soybean chromosomes. Hill et al. (2004a, b) first discovered nine SA-resistant genotypes. Of the nine genotypes, Dowling (PI 548663), Jackson (PI 548657), and ‘Sugao Zarai’ (PI 200538) were later shown as having SA resistance characterized as antibiosis by Li et al. (2004). Hill et al. (2006a, b) identified that a SA-resistance gene named Rag1 was present in Dowling and a gene named Rag was present in Jackson. These were both dominant resistance genes and they were mapped to the same region between Satt463 and Satt435 on soybean chromosome 7 [linkage group (LG) M] (Li et al. 2007). Mensah et al. (2008) identified two PIs with antibiosis resistance and two PIs with antixenosis resistance to SA. Zhang et al. (2009) further evaluated PI 567541B, one of the sources of antibiosis resistance, and mapped two recessive quantitative trait loci (QTL) from this source. They mapped one QTL onto chromosome 7 (LG M) and another onto chromosome 13 (LG F). The genetic location of the QTL on chromosome 7 was the same as the location of Rag1 from Dowling, however, the QTL on chromosome 7 was recessive in contrast to the dominant resistance found for Rag1. They also identified a significant interaction between two genes (Zhang et al. 2009). Mian et al. (2008a) identified that PI 243540, PI 567301B, and PI 567324 are resistant to SA biotype 2 collected in Ohio. Kang et al. (2008) showed that strong antibiosis resistance in PI 243540 was controlled by a single dominant gene that was mapped to chromosome 13 (LG F) by Mian et al. (2008b) and this gene was subsequently named Rag2. Hill et al. (2009) recently reported that PI 200538 carries a single dominant gene conferring resistance to both SA biotype 1 and 2 and this gene maps to the same genomic region as the Rag2 allele from PI 243540.

All publications referred to herein are incorporated by reference to the extent not inconsistent herewith for all purposes, including enablement and written description. References cited herein reflect the state of the art relevant to what is disclosed and claimed herein.

### SUMMARY

This disclosure provides nucleic acid molecules representing a genetically defined region of the genome of the aphid resistant soybean plant (Glycine max) cultivar PI 200538 that confers resistance to soybean aphid (Aphis glycines). Within the region is a gene encoding the aphid resistance protein Rag2. Also provided herein are methods for conferring aphid resistance on a plant or enhancing aphid resistance in a plant by transforming it to contain and express such nucleic acid sequences encoding Rag2 aphid resistance or introgressing DNA encoding the trait into the plant by plant breeding. Further provided are polymorphic markers useful for identifying plant germplasm containing aphid resistance, and methods for using such markers.

An isolated, synthetic or recombinant DNA molecule is provided herein selected from the group consisting of: DNA molecules having a sequence selected from the group consisting of: the Rag2 Interval nucleic acid sequence [SEQ ID NO:65]; and base pairs 1294-1696 (Gene 1); 7377-7976 (Gene 2); 9421-11097 (Gene 3); 16204-17803 (Gene 4); 19326-22276 (Gene 5); 42008-42295 (Gene 6); 43619-44180 (Gene 7); 46451-47240 (Gene 8); and 48415-48738 (Gene 9) of said SEQ ID NO:65; and RNA molecules having sequences of RNA molecule expressed by said DNA molecules; which DNA and RNA molecules encode a polypeptide that is capable of conferring aphid resistance to a soybean plant or participating in conferring or enhancing aphid resistance to a soybean plant when expressed by the plant; complements of said DNA and RNA molecules; and polypeptide molecules encoded by said DNA and RNA molecules, and sequences having about 95% to 100% homology to the foregoing. The sequence is that of aphid-resistant soybean variety PI 200538. Other aphid-resistance sequences falling within the foregoing definition are found in other aphid-resistant soybean plants such as PI 243540.

In an embodiment, the DNA molecule has the sequence of Gene 2 or Gene 3, or a sequence having about 95% to 100% homology thereto.

In embodiments, RNA and polypeptide molecules expressed by the foregoing molecules are also provided herein.

DNA molecules as described above operably linked upstream at the 5′ end to a promoter capable of causing expression of said molecule in a heterologous host plant are also provided. Constructs comprising such DNA molecules can be used to transform host cells, such as plant cells, including cells of aphid-susceptible plants including soybean. Such transformed cells can be used in methods for producing transgenic plants capable of producing aphid-resistant progeny.

In embodiments, a polypeptide synthesized using the sequences of polypeptides expressed by the nucleic acid molecules capable of conferring aphid resistance on a plant, or an isolated polypeptide expressed by such DNA molecules can be applied to a plant, such as by spraying or coating, to confer aphid resistance on the plant.

Also provided herein is a method for producing an aphid-resistant soybean crop in a field comprising planting the field with crop seeds or plants that are aphid-resistant as a result of being derived from transformed cells comprising DNA expressing the Rag2 aphid resistance gene.

Also provided herein is a method for determining the presence or absence of a gene for aphid resistance in soybean germplasm comprising analyzing said germplasm by marker-assisted selection (MAS) to (a) detect a Rag2 aphid-resistance locus that maps to soybean chromosome 13 of said soybean germplasm, wherein said Rag2 locus shows allelic polymorphism between aphid-resistant and aphid-susceptible soybean and is flanked on opposite sides by markers KS5 and KS9-3, wherein the Rag2 locus comprises allelic DNA sequences that control aphid resistance; and (b) determine the presence or absence of an allelic form of DNA linked to the Rag2 gene coding for resistance to Aphis glycines in said germplasm; said method comprising: (1) making a first PCR-amplified polymorphic marker fragment from said soybean germplasm; (2) making a second PCR-amplified polymorphic marker fragment from soybean germplasm of a plant having aphid resistance conferred by said Rag2 gene, (i) wherein the second fragment is made by PCR amplification of the same marker that was used to make said first fragment, and (ii) wherein said second fragment has a size substantially the same as that of a PCR-amplified polymorphic marker fragment of germplasm of aphid-resistant soybean variety PI200538 made using the same marker used to make said first and second fragments; wherein said gene coding for Rag2 resistance is present in said soybean germplasm when said first fragment is substantially the same size as said second fragment, and wherein said gene is not present in said germplasm when said first fragment is not substantially the same size as said second fragment.

Further provided herein is a kit for selecting at least one soybean plant by marker-assisted selection of a quantitative trait locus associated with resistance to Aphis glycines comprising: (a) primers and/or probes for detecting at least one Aphis glycines resistance-associated marker locus selected from the group consisting of primers or probes comprising at last about 10 or at least about 75 base pairs of at least two markers or genes selected from the group consisting of Markers #1, #9, #20, #23, #24. #25, #34, KS2, KS4, KS5, KS7, KS12, KS14, KS16, #1485, and KS9-3 and markers mapping within about 5 to about 10 cM thereof; and Genes 1-9; and (b) instructions for using the primers and/or probes for detecting genes and/or marker loci and correlating the genes and/or loci with predicted aphid resistance.

### BRIEF DESCRIPTION OF THE DRAWINGS

FIG. 1 provides genetic and physical maps of the interval Rag2 located on soybean chromosome 13 (LG F): (a) Linkage map of the interval Rag2 is located using the same population of 95 F2:3 lines as Hill et al. (2009). The numbers between the markers are the linkage distances in centiMorgans (cM); (b) Linkage map of the interval Rag2 is located using SNP markers developed by re-sequencing of STSs using the same Hill et al. (2009) population; (c) High-resolution physical map of the interval Rag2 is located using eight recombinant lines and SNP markers developed by re-sequencing in the target interval. Numbers between the markers show physical distances in kilo bases (kb) on the Williams 82 89 assembly (Glyma1) available at http://www.phytozome.net (Schmutz et al. 2010). The interval containing Rag2 was narrowed down to a 54-kb region between the markers KS9-3 and S5.

FIG. 2. A and B, first and last portions of the Rag2 interval from the soybean aphid (SA) resistant line. Protein-coding genes were represented with the numerical index (1 through 9, gray blocks) that was followed by the predicted gene functions. Empty triangles represent SNP marker positions flanking the Rag 2 interval.

### DETAILED DESCRIPTION

This disclosure provides an isolated, synthetic or recombinant nucleic acid molecule that is at least about or 95% to about 100%, or at least about or 90% to about 100%, or at least about or 85% to about 100%, or at least about or 80% to about 100%, or at least about 75% to about 100% (or any subrange or integer within the foregoing ranges) identical to the Glyma13g26000 nucleic acid sequence [SEQ ID NO:64] or the Rag2 interval [SEQ ID NO:65] or Genes 1-9, which encodes a polypeptide that is capable of conferring or contributing to conferring Rag2 aphid resistance to a host plant when expressed by the plant and a complement of any of the foregoing. The nucleic acid molecule codes for aphid resistance and has polymorphisms compared to the Glyma13g26000 nucleic acid sequence derived from Glycine max cv. Williams 82 aphid susceptible cultivar or a sequence identical to SEQ ID NO:65.

Also provided is an isolated, synthetic or recombinant nucleic acid molecule encoding a gene for aphid resistance that is at least about or 95% to about 100%, or at least about or 90% to about 100%, or at least about or 85% to about 100%, or at least about or 80% to about 100%, or at least about 75% to about 100% (or any subrange or integer within the foregoing ranges) identical to a gene of soybean accession Nos. PI 200538 or PI 243540 that maps between markers KS9-3 and KS5 on chromosome 13, and which encodes a polypeptide that is capable of conferring or contributing to conferring Rag2 aphid resistance to a host plant when expressed by the plant, or a complement of the foregoing.

Sequence identity can be determined using a sequence comparison algorithm comprising a BLAST program with default parameters.

Further provided herein is an isolated, synthetic or recombinant nucleic acid molecule having a sequence that encodes a polypeptide capable of conferring, or participating in conferring, aphid resistance to a soybean plant wherein the complement of said sequence hybridizes under highly stringent conditions to: (a) a second nucleic acid molecule having a sequence of SEQ ID NO:64 or 65, or (b) a third nucleic acid molecule encoding a polypeptide having an amino acid sequence of SEQ ID NO:63 (Glyma13g26000.1; Genomic Span: 4378 bp; Position: Gm13:29226207-29230584 (+strand).

Highly stringent conditions comprise a hybridization under conditions can comprise a buffer comprising 50% formamide at about 37 C to 42° C.; or, 42° C. in 50% formamide, 5×SSPE, 0.3% SDS, and a wash step comprising use of a buffer comprising 0.15 NaCl for 15 min at 72° C.

Further provided is an isolated, synthetic or recombinant nucleic acid molecule encoding a Rag2 gene from aphid-resistant soybean variety PI 200538 or PI 243540.

A fragment of any the foregoing nucleic acid molecules that is capable of conferring or participating in conferring aphid resistance on a plant into which it is transformed is also provided. The nucleic acid molecules hereof can be RNA or DNA molecules.

Provided herein is a substantially purified, isolated, recombinant or synthetic polypeptide having at least about or 95% to about 100%, or at least about or 90% to about 100%, or at least about or 85% to about 100%, or at least about or 80% to about 100%, or at least about 75% to about 100% (or any subrange or integer within the foregoing ranges) identity or similarity to a polypeptide encoded by a nucleic acid molecule of described above, wherein the polypeptide is capable of conferring or participating in conferring Aphis glycines resistance on a soybean plant. The isolated, recombinant or synthetic polypeptide can have at least about or 95% to about 100%, or at least about or 90% to about 100%, or at least about or 85% to about 100%, or at least about or 80% to about 100%, or at least about 75% to about 100% (or any subrange or integer within the foregoing ranges) identity or similarity to a polypeptide having a sequence set forth in SEQ ID NO:63 or a sequence encoded by a nucleic acid sequence of SEQ ID NO:65. The polypeptide is capable of conferring or participating in conferring aphid resistance on a plant and has sequence differences compared to the Glyma13g26000 polypeptide sequence derived from the Glycine max cv. Williams 82 aphid susceptible cultivar. Also provided herein is a fragment of such a polypeptide that is capable of conferring or contributing to conferring aphid resistance on a plant. Such polypeptides can be applied to the surfaces of plants to make them unattractive to aphids. Isolated, synthetic or recombinant nucleic acid molecules encoding such polypeptides are also provided herein.

Further provided is a recombinant host cell comprising a nucleic acid molecule as described hereinabove, wherein said nucleic acid molecule is non-native to said host cell. This recombinant cell can be a microbial cell such as a bacterial, fungal or yeast cell or it can be a transgenic plant cell including a transgenic plant cell that is part of a transgenic plant or progeny of the transgenic plant. A transgenic plant comprising a recombinant nucleic acid as described herein, made by transforming a plant susceptible to aphid infestation, which is a soybean, alfalfa, clover, pea, bean, lentil, lupin, mesquite, carob, peanut, apple, apricot, peach, pear, plum, blackberry, blueberry, strawberry, cranberry, lemon, orange, maize, wheat, rye, barley, oat, buckwheat, sorghum, rice, sunflower, canola, cotton, linseed, cauliflower, asparagus, lettuce, tobacco, mustard, sugarbeet, potato, sweet potato, carrot, turnip, celery, tomato, eggplant, cucumber, or squash. Other plants can also be transformed with the nucleic acid molecules described herein, including Solanaceae, Legumes, Brassicas and ornamental plants, for example, roses or columbines, among others.

Also provided are primers and probes useful for determining if germplasm is from a Rag2 aphid-resistant plant and developing markers mapping to the Rag2 locus on soybean chromosome 13. These are isolated, recombinant or synthetic nucleic acid molecules that are at least about or 95% to about or 100%, or at least about or 90% to about or 100%, or at least about or 85% to about or 100%, or at least about or 80% to about or 100%, or at least about 75% to about or 100% (or any subrange or integer within the foregoing ranges) identical to a nucleic acid sequence selected from the group consisting of a sequence of SEQ ID NOS:1-62, 64 and 65, and primers and probes made from sequences of at least about 10 or at least about 75 base pairs of Genes 1-9, and in embodiments, of Genes 2 or 3. Such primers and probes can be used to amplify DNA in germplasm of plants that are known or that are not known to possess Rag2 aphid resistance, and then the sequences of the amplified DNA can be compared with the sequences provided herein in methods to determine whether or not the plant possesses Rag2 resistance, e.g., by sequence analysis or fragment size analysis. In embodiments, the probe or primer consists of the first and/or last 10 to 75 consecutive base pairs of Gene 2 or Gene 3. Sequences 1-62 and 64 are from the Rag2 interval of aphid-susceptible Williams 82 soybean, while sequence 65 is from the Rag2 interval of Soybean Accession No. PI 200538, an aphid-resistant soybean. Amplified sequences identical to SEQ ID NO:65 are from aphid-resistant germplasm, while amplified sequences identical to SEQ ID NO:64 are from aphid-susceptible germplasm.

Further provided are cloning vectors, such as a viral vector, a plasmid, a phage, a phagemid, a cosmid, a fosmid, a bacteriophage, an artificial chromosome, an adenovirus vector, a retroviral vector, or an adeno-associated viral vector, as well as expression vectors capable of replicating in a host cell comprising the nucleic acid molecules described above. Host cells can be cells of prokaryotes, eukaryotes, funguses, yeasts, bacteria, plants and non-human metabolically rich hosts, and any other such host cells, capable of being transformed with the above-described nucleic acid molecules. In embodiments, the nucleic acid molecule is non-native to the host cell, such as a transgenic plant cell comprising the nucleic acid molecule.

Also provided herein is an array comprising an immobilized nucleic acid having a sequence as described above.

Transgenic plants and seeds which are progeny of the transgenic plant cells are also provided, such as aphid-resistant plants of elite lines such as elite soybean lines, or other plants transformed to contain above-described nucleic acid molecules. In embodiments the transgenic plants are made from plants that are otherwise susceptible to aphid infestation.

Methods of producing a recombinant polypeptides are provided herein, said methods comprising the steps of introducing a nucleic acid molecule described above into a host cell under conditions that allow expression of the polypeptide. The nucleic acid molecule can be linked to a promoter. The transformed cells produced by these methods can express the recombinant polypeptide.

A method of generating one or more nucleic acid molecules capable of conferring or contributing to conferring Aphis glycines resistance on a soybean or other plant is also provided, the method comprising: obtaining the nucleic acid molecule described above and modifying one or more nucleotides in said nucleic acid to another nucleotide, deleting one or more nucleotides in said nucleic acid molecule, or adding one or more nucleotides to said nucleic acid molecule to obtain a modified nucleic acid molecule, wherein the modified nucleic acid molecule encodes an polypeptide capable of conferring or contributing to conferring Aphis glycines resistance on a soybean plant. In embodiments such modified nucleic acids are tested for their ability to confer or contribute to conferring aphid resistance on a plant, and those that are capable of conferring or contributing to conferring resistance are selected. Suitable modification methods are known to the art, including: effor-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturation mutagenesis (GSSM) and any combination of these methods.

A method for comparing a first sequence of germplasm from an aphid-resistant plant to a second sequence is also provided herein, comprising the steps of: electronically encoding the first sequence and the second sequence in a computer program which compares sequences; and operating the computer program to determine differences between the first sequence and the second sequence, wherein said first sequence comprises a nucleic acid sequence as described above. In embodiments, the step of determining differences between the first sequence and the second sequence further comprises the step of identifying polymorphisms between the first and second sequences that are diagnostic and/or effective in conferring or contributing to conferring Aphis glycines resistance on a plant.

The nucleic acid molecules provided herein and fragments thereof can be used as probes and/or primers for identifying a nucleic acid encoding a polypeptide conferring or capable of contributing to conferring aphid resistance on a plant. The probe or primer can comprise at least about or 10-12 consecutive bases, and in embodiments least about or 70 or at least or about 75 consecutive bases of the nucleic acid sequences described above, including molecules having the sequences of SEQ ID NOS:1-62 and portions or all of SEQ ID NOS:64 or 65, including Genes 1-9 and portion thereof. Probes hereof are capable of identifying nucleic acid molecules encoding polypeptides described above by hybridization, such as hybridization under highly stringent conditions. Such probes can comprise at least one polymorphism that is different between corresponding sequences in aphid-resistant and aphid-susceptible plant germplasm.

The probes provided herein are useful in a method for isolating or recovering a nucleic acid encoding a polypeptide with the ability to confer or contribute to conferring aphid resistance to a plant, from a sample comprising germplasm of a plant, said method comprising. (a) providing a nucleic acid probe as described herein; (b) isolating a nucleic acid from the sample or treating the sample such that nucleic acid in the sample is accessible for hybridization to the probe; (c) combining the isolated nucleic acid or the treated sample of step (b) with the nucleic acid probe; and (d) isolating a nucleic acid molecule that specifically hybridizes with the probe, which nucleic acid molecule encodes a polypeptide having at least about or 95% to about 100%, or at least about or 90% to about 100%, or at least about or 85% to about 100%, or at least about or 80% to about 100%, or at least about 75% to about 100% (or any subrange or integer within the foregoing ranges) identity or similarity or similarity to SEQ ID NO:63 or is identical to a polypeptide encoded by a DNA molecule having the sequence of SEQ ID NO:65, and has the ability to confer or contribute to conferring aphid resistance to a plant; thereby isolating or recovering a nucleic acid molecule encoding a polypeptide with the ability to confer or contribute to conferring aphid resistance to a plant from the sample.

The nucleic acid molecules provided herein are also useful for identifying or isolating further nucleic acid molecules capable of conferring or contributing to conferring Aphis glycines resistance to a soybean plant by a method comprising hybridizing a probe comprising a nucleic acid molecule provided herein, a fragment thereof having at least about 10 or at least or about 75 base pairs, or a nucleic acid molecule having a fully complementary sequence to said nucleic acid molecule, to germplasm of a soybean. In embodiments, the hybridization is done under highly stringent hybridization conditions. The probe can further comprise a detectable isotopic label, or can comprise a detectable non-isotopic label such as a fluorescent molecule, a chemiluminescent molecule, an enzyme, a cofactor, an enzyme substrate, a hapten, and other labels known to the art.

A method for producing a recombinant polypeptide using nucleic acid molecules described above is provided comprising the steps of introducing the nucleic acid molecule encoding the polypeptide into an isolated host cell under conditions that allow expression of the polypeptide, thereby producing a recombinant polypeptide.

A gene capable of conferring or contributing to conferring aphid resistance to a plant transformed to contain said gene, wherein said gene comprises a nucleic acid molecule encoding one or more polypeptides capable of conferring or contributing to conferring aphid resistance on a plant, and encoded by DNA having the sequence selected from that of Genes 1-9 is also provided. In embodiments, the DNA has a sequence of Genes 2 or 3.

Further provided is a computer storage medium having recorded thereon a sequence selected from the group consisting of SEQ ID NOS:1-65 and Genes 1-9, together with information identifying each of said sequences.

A method of selecting a plant or plant germplasm with aphid resistance from one or more plants or plant germplasms is provided. The method comprises: (a) detecting, by marker-assisted selection (MAS), in the plant(s) or germplasm(s) the presence of at least one allele in at least one marker locus that is associated with an aphid resistance locus flanked by KS5 and KS9-3 or markers mapping within about 5 cM or about 10 cM thereof; and (b) selecting the plant(s) or germplasm(s) comprising the at least one allele in said at least one marker locus, thereby selecting a plant having aphid resistance. In embodiments, the plant or germplasm is a soybean plant or germplasm.

In embodiments, the plant or germplasm is from a plant species or genus having members which are aphid-susceptible. The presence of the marker locus, in embodiments, is determined by means of a marker provided herein, such as a marker selected from the group consisting of markers #1, #9, #20, #23, #24, #25, #34, KS2, KS4, KS5, KS7, KS12, KS14, KS16, #1485, and KS9-3, and markers mapping within about 5 cM to about 10 cM thereof, or other markers made by methods described herein using sequences from SEQ ID NO:65, and Genes 1-9. In embodiments, the presence of at least one allele associated with aphid resistance on soybean chromosome 13 is detected in a DNA interval having the sequence of SEQ ID NO:65, or Genes 1-9. In embodiments the presence of said allele is detected in Gene 2 or 3. This method is useful as part of further breeding to improve a plant's resistance to Aphis glycines. The further breeding can include the steps of: crossing a plant selected by said method to have aphid resistance with other lines or hybrids to form first progeny plants, backcrossing said plant with said first progeny plants or progeny of said first progeny plants, and/or self-crossing said plants, and combinations thereof. Plants selected by the foregoing methods are also provided herein.

A method is disclosed herein for producing one or more primers for markers associated with Rag2 Aphis glycines resistance, said method comprising: (a) providing a first nucleotide sequence of soybean chromosome 13 from a plant having Rag2 resistance to Aphis glycines wherein said first nucleotide sequence maps to a DNA interval between markers KS9-3 and KS5, or between markers within about 5 to about 10 cM of either or both of said markers; (b) providing a second nucleotide, sequence corresponding to said first sequence from a plant known to lack Rag2 Aphis glycines resistance; (c) selecting at least one forward and reverse marker primer pair with oligonucleotide lengths between at least about 10 and at least about, or about, 75 to 100 base pairs from said first nucleotide sequence; (d) separately amplifying genomic DNA from said primers in media containing said susceptible and said resistant plants, respectively, to form amplification products; (e) selecting amplification products which are the only amplification products produced by said primers in each medium; (f) determining the presence of polymorphisms between the selected amplification products from the susceptible and resistant soybean DNA; and (g) selecting primers that produce polymorphic amplification products as primers for markers associated with Rag2 aphid resistance. In embodiments, the plant is a soybean or other aphid-susceptible plant. The presence of polymorphisms can determined by direct sequencing or by melt-curve analysis or other means known to the art. Typically, the amplification products are between about 200 and 1000 kb in length. The probe sequence can be selected from a DNA interval between said primer sequences containing a said polymorphism.

Also provided are nucleic acid probes having a nucleotide sequence comprised in a nucleic acid molecule as described above, and comprising at least one polymorphism compared to a corresponding sequence from a soybean variety not having Rag2 resistance.

Abbreviations used herein include: INDEL Insertion and deletion; kb Kilobase pair; MAS Marker-assisted selection; MCA Melting curve assay; NBS-LRR Nucleotide-binding site-leucine-rich repeat; SNP Single nucleotide polymorphism; STS Sequence-tagged site; SA soybean aphid.

### The PI200538 Aphid Resistance Gene is Referred to as Rag2 Herein.

The aphid-resistance gene sequence or a subsequence thereof or a sequence complementary to such sequence or subsequence can be fully or partially chemically synthesized by means known to the art, e.g., as described in Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989); or Ausubel 1993, Current Protocols in Molecular Biology, Wiley, NY. DNA sequences can be synthesized by phosphoramidite chemistry in an automated DNA synthesizer. In addition, a sequence of the aphid sensitive coding sequence can be modified, for example, by site directed mutagenesis techniques such as mutagenic polymerase chain reaction, or by transformation with a mutagenic oligonucleotide to achieve the desired result.

The DNA constructs of the invention can be used to transform any type of plant cells. DNA segments encoding a specific gene can be introduced into recombinant host cells and employed for expressing a specific structural or regulatory protein. Alternatively, through the application of genetic engineering techniques, subportions or derivatives of selected genes can be employed. Upstream regions containing regulatory regions such as promoter regions can be isolated and subsequently employed for expression of the selected gene.

Where an expression product is to be generated, the nucleic acid sequences can be varied so long as they retain the ability to encode the same product. Reference to the known codon preferences permit those of skill in the art to design any nucleic acid encoding for the product of a given nucleic acid in a desired host.

A genetic marker can be used for selecting transformed plant cells (“a selection marker”). Selection markers typically allow transformed cells to be recovered by negative selection (i.e., inhibiting growth of cells that do not contain the selection marker) or by screening for a product encoded by the selection marker. In certain embodiments, DNA fragments can be introduced into the cells of interest by the use of a vector, so as to bring the incorporation into the genome, replication and/or expression to the attached segment. A vector can have one or more restriction endonuclease recognition sites at which the DNA sequences can be cut in a determinable fashion without loss of an essential biological function of the vector. Vectors can further provide primer sites (e.g. for PCR), transcriptional and/or translational initiation and/or regulation sites, recombinational signals, replicons, selectable markers, etc. Examples of vectors include plasmids, phages, cosmids, phagemid, yeast artificial chromosome (YAC), bacterial artificial chromosome (BAC), human artificial chromosome (HAC), virus, virus based vector, and other DNA sequences which are able to replicate or to be replicated in vitro or in a host cell, or to convey a desired DNA segment to a desired location within a host cell. Polynucleotides can be joined to a vector containing a selectable marker for propagation in a host. If the vector is a virus, it can be packaged in vitro using an appropriate packaging cell line and then transduced into host cells. If the vector is a virus, it can be packaged in vitro using an appropriate packaging cell line and then transduced into host cells.

As indicated, the expression vectors can include at least one selectable marker. Exemplary markers can include, but are not limited to, G418, glutamine synthase, herbicide resistance or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g.; Saccharomyces cerevisiae or Pichia pastoris (ATCC Accession No. 201178)); insect cells such as Drosophila S2 and Spodoptera Sf9 cells; and plant cells. Appropriate culture media and conditions for the particular host cells are known in the art.

Polynucleotide inserts can be operatively linked to an appropriate promoter, such as the phage lambda PL promoter, the E. coli lac, trp, phoA and tac promoters, and the plant-expressible promoters disclosed herein and/or known to the art. The expression constructs can further contain sites for transcription initiation, termination, and, in the transcribed region, a ribosome binding site for translation. The coding portion of the transcripts expressed by the constructs can include a translation initiating codon at the beginning and a termination codon (UAA, UGA or UAG) appropriately positioned at the end of the sequence to be translated.

In embodiments hereof, various whole-genome methods can be used to analyze nucleic acids. The methods usually in involve the detection of hybridization of genetic segments to detect the presence and level of the segments in the sample. Microarrays can be used, either spotted or synthesized on a surface. Methods involving beads, microbeads, magnetic beads or fiber bundles can also be employed. Commercial whole-genome gene expression microarrays can be obtained from Applied Biosystems, Affymetrix, Agilent, GE Healthcare, and Illumina.

Procedures used to detect the presence of nucleic acids capable of hybridizing to the detectable probe include well known techniques such as Southern blotting, Northern blotting, dot blotting, colony hybridization, plaque hybridization, and PCR. In some applications, the nucleic acid capable of hybridizing to the labeled probe can be cloned into vectors such as expression vectors, sequencing vectors, or in vitro transcription vectors to facilitate the characterization and expression of the hybridizing nucleic acids in the sample. For example, such techniques can be used to isolate and clone sequences in a genomic library or cDNA library which are capable of hybridizing to the detectable probe as described herein. A commonly used selectable marker gene for plant transformation is neomycin phosphotransferase II (nptII) which, when placed under the control of plant expression control signals, confers resistance to kanamycin. Fraley et al., Proc. Natl. Acad. Sci. USA, 80:4803 (1983). Another selectable marker gene is the hygromycin phosphotransferase gene which confers resistance to the antibiotic hygromycin. Vanden Elzen et al., Plant Mol. Biol., 5:299 (1985). Additional selectable marker genes of bacterial origin that confer resistance to antibiotics include gentamycin acetyl transferase, streptomycin phosphotransferase, aminoglycoside-3′-adenyl transferase, and the bleomycin resistance determinant (Hayford et al. 1988. Plant Physiol. 86:1216, Jones et al. 1987. Mol. Gen. Genet. 210:86; Svab et al. 1990. Plant Mol. Biol. 14:197, Hille et al. 1986. Plant Mol. Biol. 7:171). Other selectable marker genes confer resistance to herbicides such as glyphosate, glufosinate or bromoxynil (Comai et al. 1985. Nature 317:741-744, Stalker et al. 1988. Science 242:419-423, Hinchee et al. 1988. Bio/Technology 6:915-922, Stalker et al. 1988. J. Biol. Chem. 263:6310-6314, and Gordon-Kamm et al. 1990. Plant Cell 2:603-618). Other selectable markers useful for plant transformation include, without limitation, mouse dihydrofolate reductase, plant 5-enolpyruvylshikimate-3-phosphate synthase, and plant acetolactate synthase (Eichholtz et al. 1987. Somatic Cell Mol. Genet. 13:67, Shah et al. 1986. Science 233:478, Charest et al. 1990. Plant Cell Rep. 8:643; EP 154,204). Commonly used reporters for screening presumptively transformed cells include but are not limited to β-glucuronidase (GUS), β-galactosidase, luciferase, and chloramphenicol acetyltransferase (Jefferson, R. A. 1987. Plant Mol. Biol. Rep. 5:387, Teeri et al. 1989. EMBO J. 8:343, Koncz et al. 1987. Proc. Natl. Acad. Sci. USA 84:131, De Block et al. 1984. EMBO J. 3:1681), green fluorescent protein (GFP) (Chalfie et al. 1994. Science 263:802, Haseloff et al. 1995. TIG 11:328-329 and PCT application WO 97/41228). Another approach to the identification of relatively rare transformation events has been use of a gene that encodes a dominant constitutive regulator of the Zea cans anthocyanin pigmentation pathway (Ludwig et al. 1990. Science 247:449).

For applications in which the nucleic acid segments of the present invention are incorporated into vectors, such as plasmids, these segments can be combined with other DNA sequences, such as promoters, polyadenylation signals, restriction enzyme sites, multiple cloning sites, other coding segments, and others known to the art, such that their overall length can vary considerably. It is contemplated that a nucleic acid fragment of almost any length can be employed, with the total length preferably being limited by the ease of preparation and use in the intended recombinant DNA protocol.

Plasmid preparations and replication means are well known in the art. See for example, U.S. Pat. Nos. 4,273,875 and 4,567,146 incorporated herein their entirety. Some embodiments of the present invention include providing a portion of genetic material of a target cell and inserting the portion of genetic material of a target cell into a plasmid for use as an internal control plasmid.

By knowing the nucleotide sequences of Rag2 genetic material in a cell and in an internal control, specific primer sequences can be designed. In an embodiment, at least one primer of a primer pair used to amplify a portion of genomic material of a cell is in common with one of the primers of a primer pair used to amplify a portion of genetic material of an internal control such as an internal control plasmid. In an embodiment, a primer is about, but not limited to 5 to about 50 oligonucleotides long, or about 10 to 40 oligonucleotides long or about 10 to about 30 oligonucleotides long. A number of template-dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety. Suitable primer sequences for amplification can be readily synthesized by one skilled in the art or are readily available from third party providers such as Life Technologies, Inc., Integrated DNA Technologies, and Qiagen Operon, and other suppliers known to the art. Other reagents, such as DNA polymerases and nucleotides, which are useful for nucleic acid sequence amplification such as PCR are also commercially available. Nucleic acids used as a template for amplification can be isolated from cells contained in a biological sample, according to standard methodologies. The nucleic acid can be genomic DNA or fractionated or whole cell RNA. Where RNA is used, it can be desired to convert the RNA to a complementary cDNA. In one embodiment, the RNA is whole cell RNA and is used directly as the template for amplification.

Pairs of primers that selectively hybridize to nucleic acids corresponding to specific markers are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection can be performed by visual means. Alternatively, the detection can involve indirect identification of the product via chemiluminescence, radioactive scintilography of incorporated radiolabel or fluorescent label or even via a system using electrical or thermal impulse signals (Affymax technology; Bellus, 1994).

A reverse transcriptase PCR amplification procedure can be performed in order to quantify the amount of mRNA amplified. Methods of reverse transcribing RNA into cDNA are well known. Alternative methods for reverse transcription utilize thermostable DNA polymerases. These methods are described in WO 90/07641 filed Dec. 21, 1990. Polymerase chain reaction methodologies are well known in the art. Other amplification methods are known in the art besides PCR such as LCR (ligase chain reaction), disclosed in European Application No. 320 308, incorporated herein by reference in its entirety.

An isothermal amplification method, in which restriction endonucleases and ligases are used to achieve the amplification of target molecules that contain nucleotide 5′-[alpha-thio]-triphosphates in one strand of a restriction site can also be useful in the amplification of nucleic acids herein. Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated. Still other amplification methods known in the art can be used with the methods described herein.

Following amplification, it can be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989. Alternatively, chromatographic techniques can be employed to effect separation of amplified product or other molecules. There are many kinds of chromatography which can be used: adsorption, partition, ion-exchange and molecular sieve, and many specialized techniques for using them including column, paper, thin-layer and gas chromatography.

Amplification products should be visualized (detected) in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation. Probes can be labeled with radioactive, fluorescent or other labels known to the art. In an embodiment hereof, the described methods use a fluorescence resonance energy transfer (FRET) labeled probe as an internal hybridization probe. In an embodiment, an internal hybridization probe is included in the PCR reaction mixture so that product detection occurs as the PCR amplification product is formed, thereby reducing post-PCR processing time. Roche Lightcycler PCR instrument (U.S. Pat. No. 6,174,670) or other real-time PCR instruments can be used in this embodiment of the present invention, e.g., see U.S. Pat. No. 6,814,934. PCR amplification of a genetic material increases the sensitivity. In some instances, real-time PCR amplification and detection significantly reduce the total assay time so that test results can be obtained in about 12 hours. Accordingly, methods herein provide rapid and/or highly accurate results relative to the conventional methods and these results are verified by an internal control. In embodiments involving hybridization, one can employ nucleic acid sequences or fragments or complements thereof as disclosed herein in combination with a detectable signal, such as a label, for determining hybridization. A wide variety of appropriate detectable agents are known in the art, including fluorescent, radioactive, enzymatic or other ligands, such as avidin/biotin, which are capable of being detected. One can employ a fluorescent label or an enzyme tag such as urease, alkaline phosphatase or peroxidase, instead of radioactive or other environmentally undesirable reagents. In the case of enzyme tags, calorimetric indicator substrates are known which can be employed to allow detection visible to the human eye or spectrophotometrically, to identify specific hybridization with complementary nucleic acid-containing samples.

In an embodiment, visualization is achieved indirectly. Following separation of amplification products, a labeled, nucleic acid probe is brought into contact with the amplified marker sequence. The probe can be conjugated to a chromophore or can be radiolabeled. In another embodiment, the probe is conjugated to a binding partner, such as an antibody or biotin, where the other member of the binding pair carries a detectable moiety. In one embodiment, detection is by Southern blotting and hybridization with a labeled probe. The techniques involved in Southern blotting are well known to those of skill in the art and can be found in many standard books on molecular biological protocols.

Embodiments hereof include providing conditions that facilitate amplification of at least a portion of a target genetic material. However, it should be appreciated that the amplification conditions are not necessarily 100% specific. The embodiments include any method for amplifying at least a portion of a cell's genetic material (such as polymerase chain reaction (PCR), real-time PCR (RT-PCR), and NASBA (nucleic acid sequence based amplification)). In an embodiment, real time PCR (RT-PCR) is the method used for amplifying at least a portion of a cell's genetic material while simultaneously amplifying an internal control for verification of the outcome of the amplification of a cell's genetic material.

Amplification of a genetic material, e.g. DNA, is well known in the art. Methods include providing conditions that would allow co-amplification of an internal control portion of a cell's genetic material and a portion of the cell's genetic material of a test sample, if the target sequence is present in the sample. In this manner, detection of the amplification products by a specific probe for each product of the internal control portion of a cell's genetic material and a portion of the cell's genetic material is indicative of the presence of the Rag2 sequence in the sample and that the conditions for the amplification are working.

Thus, a negative result indicative of absence of a target Rag2 sequence can be confirmed. Typically, to verify the working conditions of PCR techniques, positive and negative external controls are performed in parallel reactions to the sample tubes to test the reaction conditions, for example using a control nucleic acid sequence for amplification. In some embodiments, an internal control can be used to determine if the conditions of the RT-PCR reaction is working in a specific tube for a specific target sample. Alternatively, in some embodiments, an internal control can be used to determine if the conditions of the RT-PCR reaction are working in a specific tube at a specific time for a sample.

The presence or absence of PCR amplification product can be detected by any of the techniques known to one skilled in the art. In one particular embodiment, methods of the present invention include detecting the presence or absence of the PCR amplification product using a probe that hybridizes to a particular Rag2 sequence. By designing the PCR primer sequence and the probe nucleotide sequence to hybridize different portions of the Rag2 genetic material, one can increase the accuracy and/or sensitivity of the methods disclosed herein.

In general, it is envisioned that the hybridization probes described herein are useful both as reagents in solution hybridization, as in PCR, for detection of presence of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions depend on the particular circumstances based on the particular criteria required (depending, for example, on the G+C content, type of target nucleic acid, source of nucleic acid, size of hybridization probe, and other conditions known to the art). Following washing of the hybridized surface to remove non-specifically bound probe molecules, hybridization is detected, or even quantified, by means of the label.

Methods disclosed herein are not limited to the particular probes disclosed and particularly are intended to encompass at least nucleic acid sequences that are hybridizable to the disclosed sequences or are functional sequence analogs of these sequences. For example, a partial sequence can be used to identify a structurally-related gene or the full length genomic or cDNA clone from which it is derived. Those of skill in the art are well aware of the methods for generating cDNA and genomic libraries which can be used as a target for the above-described probes.

Certain embodiments involve incorporating a label into a probe, primer and/or target nucleic acid to facilitate its detection by a detection unit. A number of different labels can be used, such as Raman tags, fluorophores, chromophores, radioisotopes, enzymatic tags, antibodies, chemiluminescent, electroluminescent, affinity labels, etc. One of skill in the art will recognize that these and other label moieties not mentioned herein can be used in the disclosed methods.

Fluorescent labels of use can include, but are not limited to, Alexa 350, Alexa 430, AMCA (7-amino-4-methylcoumarin-3-acetic acid), BODIPY (5,7-dimethyl-4-bora-3a,4a-diaza-s-indacene-3-propionic acid) 630/650, BODIPY 650/665, BODIPY-FL (fluorescein), BODIPY-R6G (6-carboxyrhodamine), BODIPY-TMR (tetramethylrhodamine), BODIPY-TRX (Texas Red-X), Cascade Blue, Cy2 (cyanine), Cy3, Cy5,6-FAM (5-carboxyfluorescein), Fluorescein, 6-JOE (2′7′-dimethoxy-4′5′-dichloro-6-carboxyfluorescein), Oregon Green 488, Oregon Green 500, Oregon Green 514, Pacific Blue, Rhodamine Green, Rhodamine Red, ROX (6-carboxy-X-rhodamine), TAMRA (N,N,N′,N′-tetramethyl-6-carboxyrhodamine), Tetramethylrhodamine, and Texas Red. Fluorescent or luminescent labels can be obtained from standard commercial sources, such as Molecular Probes (Eugene, Oreg.). Examples of enzymatic labels include urease, alkaline phosphatase or peroxidase. Colorimetric indicator substrates can be employed with such enzymes to provide a detection means visible to the human eye or spectrophotometrically. Radioisotopes of potential use include ¹⁴-carbon, ³hydrogen, ¹²⁵iodine, ³²phosphorous, ³³phosphorous and ³⁵sulphur.

As described herein, an aspect of the present disclosure concerns isolated nucleic acids and methods of use of isolated nucleic acids. In certain embodiments, the nucleic acid sequences disclosed herein have utility as hybridization probes or amplification primers. These nucleic acids can be used, for example, in diagnostic evaluation of plant tissue samples. In certain embodiments, these probes and primers consist of oligonucleotide fragments. Such fragments should be of sufficient length to provide specific hybridization to an RNA or DNA tissue sample. The sequences typically will be 10-20 nucleotides, but can be longer. Longer sequences, e.g., 40, 50, 100, 500 and even up to full length, can be used for certain embodiments.

Nucleic acid molecules having contiguous stretches of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75 80, 85, 90, 95, 100, 125, 150, 175, 200, 250, 300, 400, 500, 600, 750, 1000, 1500, 2000, 2500 or more nucleotides from a sequence selected from the nucleic acid sequences set forth herein are contemplated. Molecules that are complementary to the above-mentioned sequences and that bind to these sequences under high stringency conditions also are contemplated. These probes are useful in a variety of hybridization embodiments, such as Southern and Northern blotting.

The use of a hybridization probe of between about 14 and 100 nucleotides in length allows the formation of a duplex molecule that is both stable and selective. Molecules having complementary sequences over stretches greater than 20 bases in length are generally preferred in order to increase stability and selectivity of the hybrid and thereby improve the quality and degree of particular hybrid molecules obtained. One generally designs nucleic acid molecules having stretches of 20 to 30 nucleotides, or even longer where desired by directly synthesizing the fragment by chemical means or by introducing selected sequences into recombinant vectors for recombinant production.

Accordingly, the nucleotide sequences herein can be used for their ability to selectively form duplex molecules with complementary stretches of genes or RNAs or to provide primers for amplification of DNA or RNA from tissues. Depending on the application envisioned, one can desire to employ varying conditions of hybridization to achieve varying degrees of selectivity of probe towards target sequences.

### DEFINITIONS

As used herein, the terms “recombinant polynucleotide” and “recombinant nucleic acid molecule.” are used interchangeably to refer to linear or circular, purified or isolated polynucleotides that have been artificially designed and which comprise at least two nucleotide sequences that are not found as contiguous nucleotide sequences in their initial natural environment. As noted above, this disclosure provides a recombinant nucleic acid construct or expression vector that facilitates the expression of the Rag2 nucleic acid sequence discussed herein in plants. As used herein, the term “nucleic acid construct” (or “DNA construct”) refers to nucleic acid fragments assembled through genetic engineering techniques operatively linked in a functional manner to direct the expression of a nucleic acid sequence of interest, such as the Rag2 nucleic acid sequence discussed herein. The construct can also include additional sequence(s) or gene(s) of interest. As used herein, the terms “recombinant polynucleotide” and “recombinant nucleic acid molecule,” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

As used herein, “nucleic acid” molecules refer to DNA and RNA molecules.

The terms “Aphis glycines” and “aphid” are used synonymously herein.

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the considered polynucleotide.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

The terms “participating in conferring” and “contributing to conferring” with respect to polynucleotides and polypeptides that can confer or help confer Aphis glycines resistance on a plant mean that aphid resistance already present in the plant is enhanced, or that the sequences provided herein can cooperate with other sequences already present in the plant or inserted into the plant genome to confer or enhance aphid resistance in the plant, or can be triggered by environmental conditions in order to confer aphid resistance to the plant, such as by being linked to promoters that respond to such environmental conditions. The terms “participating in conferring” and “contributing to conferring” are used synonymously herein and are not meant to indicate that polynucleotides or polypeptides that contribute or participate in conferring resistant must necessarily interact.

The term “array” includes a device known to the art as a “microarray” or “biochip” or “chip” that comprises a plurality of target elements, each target element comprising a defined amount of one or more polypeptides (including antibodies) or nucleic acids immobilized onto a defined area of a substrate surface, for example as defined in U.S. Pat. No. 7,592,434, incorporated herein by reference to the extent not inconsistent herewith.

The terms “computer,” “computer program” and “processor” are used herein in their broadest general contexts and incorporate all such devices known to the art.

A “coding sequence of or a “sequence that encodes” a particular polypeptide or protein, is a nucleic acid sequence that is capable of being is transcribed and translated into the polypeptide or protein when placed under the control of appropriate regulatory sequences.

Sequence “homology,” “identity,” and “similarity” can be measured using sequence analysis software, known to the art, for example, as described in U.S. Pat. No. 7,592,434. Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence, e.g., a sequence described and claimed herein, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window”, as used herein, includes reference to a segment of any one of the numbers of contiguous residues. For example, in aspects hereof, contiguous residues ranging anywhere from 20 bp or amino acid residues to the full length of an exemplary polypeptide or nucleic acid sequence of the invention are compared to a reference sequence of the same number of contiguous positions after the two sequences are optimally aligned. If the reference sequence has the requisite sequence identity to an exemplary polypeptide or nucleic acid sequence described herein, e.g., 85%, 86%, 87%, 88%, 89%, 90%, 91%, 92%, 93%, 94%, 95%, 96%, 97%, 98%, 99%, or more sequence identity to a sequence hereof, that sequence is within the scope of the claims hereof so long as it is capable performing the function(s) recited in the claims. In alternative embodiments, subsequences ranging from about 20 bp to 600 bp, about 50 bp or amino acid residues to 200 bp or amino acid residues, and about 100 to 150 bp or amino acid residues are compared to a reference sequence of the same number of contiguous positions after the two sequences are optimally aligned. Methods of alignment of sequence for comparison are well known in the art.

As used herein, the term “sequence identity” refers to amino acid or nucleic acid sequences that, when compared using the local homology algorithm of Smith and Waterman in the BestFit program (Wisconsin Package Version 10.0, Genetics Computer Group (GCG), Madison, Wis., 1981), are exactly alike. “Sequence identity” between two nucleotide or protein sequences can be determined by using programs such as a BLAST program (Altschul et al., Nucleic Acids Res. 25:3389 3402; 1997) using the following parameters:
p Program Name=blastx, blastn or blastp
d Database=nr
e Expectation value (E)=10
F Filter query sequence (DUST with blastn, SEG with others=T
G Cost to open a gap=−1
E Cost to extend a gap=−1
X X dropoff value for gapped alignment (in bits)=blastn 30, tblastx 0, all others -q Penalty for a nucleotide mismatch (blastn only)=−3
r Reward for a nucleotide match (blastn only)=1
f Threshold for extending hits, defaultblastp 11, blastn 0, blastx 12, tblastn 13 tblastx 13-g Perform gapped alignment (not available with tblastx)=T−Q Query Genetic code to use=1−D DB Genetic code (for tblast[nx] only)=1
M Matrix=BLOSUM62-W Word size=blastn 11, megablast 28, all others 3-z Effective length of the database (use zero for the real size)=0−K Number of best hits from a region to keep. As is known to one of skill in the art, other default settings can be used.

As used herein, the term “sequence similarity” refers to amino acid sequences that, when compared using the local homology algorithm of Smith and Waterman in the BestFit program (Wisconsin Package Version 10.0, Genetics Computer Group (GCG), Madison, Wis. 1981), match when conservative amino acid substitutions are considered.

As used herein to describe sequences capable of conferring aphid resistance on a plant, the term “at least about 100%” with respect to identity to SEQ ID NOS:63 and 64 from Glycine Max cv. Williams 82 means sequences having less than 100% identity to these amino acid and nucleic acid coding sequences. Sequence differences that are found in the corresponding sequences from aphid resistance cultivars are required to confer the claimed ability of the sequences to contribute to or confer aphid resistance on a plant.

As used herein, a “coding sequence,” “structural nucleotide sequence” or “gene” or “structural gene” is a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, and recombinant nucleotide sequences.

The “complement” of a nucleic acid sequence as used herein is a nucleic acid sequence that forms a double-stranded structure with another nucleic acid fragment by following base-pairing rules (for DNA, A pairs with T and C with G; for RNA A pairs with U and C pairs with G). The complementary sequence to GTAC for example, is CATG.

As used herein, a “recombinant” DNA, structure, or organism is one that does not exist in nature, and is produced only by genetic engineering techniques, such as isolation of nucleic acid, transforming an organism with DNA that the organism does not naturally contain, or does not naturally contain in the location where it is placed, synthesizing nucleic acids, adding to or deleting nucleic acids from pre-existing nucleic acids, mutating nucleic acids in vivo or in vitro, or artificially producing the “recombinant” element by any means known to the art.

As used herein, “expression” refers to the transcription and stable accumulation of mRNA derived from nucleic acid molecules or regions, e.g., nucleic acid molecules or regions described herein. “Expression” can also refer to translation of mRNA into a polypeptide. Provided are DNA constructs comprising the aphid resistance coding sequence hereof operatively linked to plant gene expression control sequences. “DNA constructs” are defined herein to be constructed (non-naturally occurring) DNA molecules useful for introducing DNA into host cells, and the term includes chimeric genes, expression cassettes, and vectors.

As used herein “operatively linked” refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art.

“Expression control sequences” are DNA sequences involved in any way in the control of transcription or translation. Suitable expression control sequences and methods of making and using them are well known in the art and can be used with plants transformed to contain the nucleic acid constructs disclosed herein. The expression control sequences should include a promoter. The promoter can be any DNA sequence which shows transcriptional activity in the chosen plant cells, plant parts, or plants. The promoter can be inducible or constitutive. It can be naturally-occurring, can be composed of portions of various naturally-occurring promoters, or can be partially or totally synthetic. Guidance for the design of promoters is provided by studies of promoter structure, such as that of Harley and Reynolds, Nucleic Acids Res., 15, 2343-61 (1987). Also, the location of the promoter relative to the transcription start can be optimized. Many suitable promoters for use in plants are well known in the art.

As used herein, a “genotype” refers to the genetic constitution, latent or expressed, of all the genes present in an individual organism such as a plant. As used herein, a “phenotype” of an organism such as a plant is any of one or more characteristics of a plant (e.g. male sterility, yield, quality improvements, etc.), as contrasted with the genotype. A change in genotype or phenotype can be transient or permanent.

As used herein, an “analog” of a first nucleotide sequence refers to a second nucleic acid sequence that is functionally the same as the first nucleotide sequence. For example, an “analog” of the Rag2 nucleic acid sequence is a nucleotide sequence from a plant species that encodes a polypeptide that is functionally equivalent to the polypeptide expressed by the Rag2 nucleic acid sequence in conferring aphid resistance on a plant carrying it and that has substantial amino acid sequence identity or similarity to the Rag2 polypeptide from soybean, for example at least about or 95% or at least about or 97% or at least about or 98% or at least about or 99% sequence identity or similarity.

As used herein, “hybridization” with respect to nucleic acids refers to a strand of nucleic acid joining with a complementary strand via base pairing. Hybridization occurs when complementary sequences in the two nucleic acid strands bind to one another.

As used herein, an “isolated” nucleic acid molecule is one that is separate from or purified away from other nucleic acid sequences in the cell of the organism in which the nucleic acid naturally occurs, i.e., such as by conventional nucleic acid-purification methods. The term embraces naturally-occurring nucleic acid sequences, recombinant nucleic acid sequences and chemically synthesized nucleic acid sequences.

As used herein, the term “isolated polypeptide” refers to a polypeptide separate from other polypeptides that are naturally present in an organism or cell, e.g., produced by expression of an isolated nucleic acid molecule described herein or produced by chemical synthesis. The term can also refer to a polypeptide that has been sufficiently separated from other polypeptides or proteins with which it would naturally be associated, so as to exist in substantially pure form.

As used herein, a “functionally equivalent fragment” of a larger polypeptide refers to a polypeptide that lacks at least one residue of the larger polypeptide, e.g., lacks at least one residue from an end of the larger polypeptide. Such a fragment retains a functional activity of the full-length polypeptide when expressed in a transgenic plant and/or possesses a characteristic functional domain or an immunological determinant characteristic of the native larger polypeptide. Immunologically active fragments typically have a minimum size of 7 or 17 or more amino acids, for example 10 amino acids. Useful Rag2 fragments are generally at least 10 amino acids in length.

As used herein, “combinations of” polypeptide fragments or “combinations of nucleotide sequences encoding combinations of polypeptide fragments” can refer to separate fragments or to single nucleotide or polypeptide molecules in which the component fragments are bonded together, either in the order in which they occur naturally in the Rag2 interval, or in any rearranged order that functions to produce aphid resistance in a transformed plant.

As used herein, the term “native” with respect to a nucleic acid sequence or polypeptide refers to a naturally-occurring (“wild type”) nucleic acid sequence or polypeptide.

As used herein, a “percentage of sequence identity” is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the window of comparison and multiplying the result by 100 to yield the percentage of sequence identity. The percentage of sequence identity can be determined by using programs such as a BLAST program (Altschul et al., Nucleic Acids Res. 25:3389 3402, 1997) using the default parameters.

As used herein, “plant” means plant cells, plant protoplast, plant cell or tissue culture from which soybean plants can be regenerated, plant calli, plant clumps and plant cells that are intact in plants or parts of plants, such as seeds, pods, flowers, cotyledons, leaves, stems, buds, roots, root tips and other suitable plant parts.

As used herein, a “polymorphism” is a change or difference between two related nucleic acids. A “nucleotide polymorphism” refers to a nucleotide which is different in one sequence when compared to a related sequence when the two nucleic acids are aligned for maximal correspondence. A “genetic nucleotide polymorphism” refers to a nucleotide which is different in one sequence when compared to a related sequence when the two nucleic acids are aligned for maximal correspondence, where the two nucleic acids are genetically related, i.e., homologous, for example, where the nucleic acids are isolated from different varieties of a soybean plant, or from different alleles of a single variety.

“Marker assisted selection” means the process of selecting a desired trait or desired traits in a plant or plants by detecting one or more nucleic acids from the plant, where the nucleic acid is linked to the desired trait.

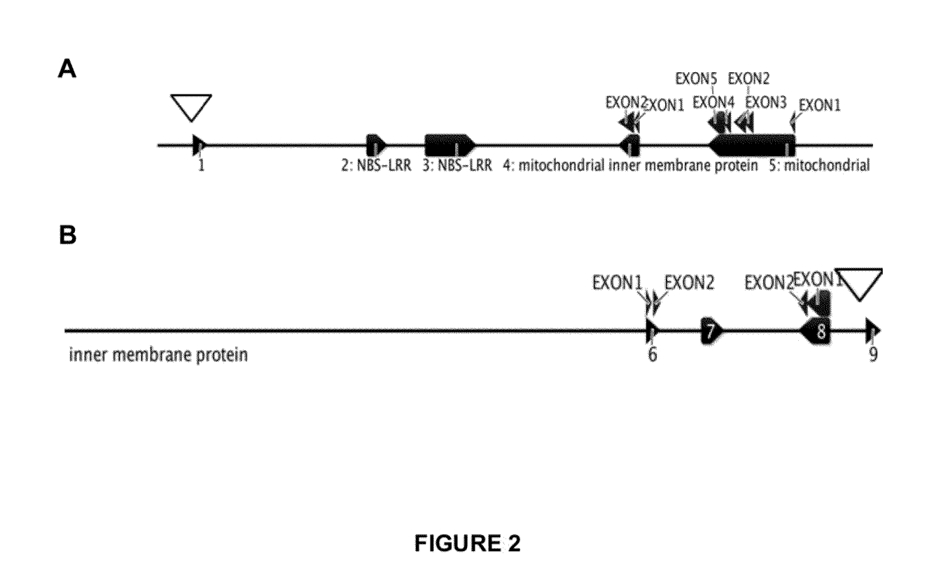

As used herein, “trait locus” refers to a chromosomal region where contains or is genetically linked (e.g., maps close to, such as within about 5 cM or about 10 cM of) a selected polymorphic nucleic acid or trait determinant. A “marker” for a particular trait is a DNA sequence that hybridizes to a trait locus. A “marker locus” is the location in DNA of an organism that hybridizes to an amplified DNA sequence, e.g., a PCR-amplified DNA sequence made from primers having a sequence in or mapping near the trait locus that contains a polymorphic trait determinant. Two loci or nucleic acid sequences on a chromosome are “genetically linked” when there is limited recombination between them during breeding. For example, if two loci are 5 cM apart on a linkage map such as shown in FIG. 2, there is a 5% chance that they will be separated by recombination during breeding. If they are 10 cM apart, there is a 10% chance they will be separated by recombination during breeding.

As used herein, a “promoter” refers to a DNA sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a DNA sequence that can stimulate promoter activity and can be an innate element of the promoter or a heterologous element inserted to enhance the expression level or tissue-specificity of a promoter. Promoters can be derived in their entirety from a native gene or be composed of different elements derived from different promoters found in nature, and/or can comprise synthetic DNA segments. It is understood by those skilled in the art that different promoters can direct the expression of a gene in different tissues or cell types, or at different stages of development, or in response to different environmental conditions. Promoters that cause a gene to be expressed in most cell types at most times are commonly referred to as “constitutive” promoters. Promoters that cause conditional expression of a structural nucleotide sequence under the influence of changing environmental conditions or developmental conditions are commonly referred to as “inducible promoters.”

For instance, suitable constitutive promoters for use in plants include promoters from plant viruses, such as the peanut chlorotic streak caulimovirus (PCISV) promoter (U.S. Pat. No. 5,850,019), the 35S promoter from cauliflower mosaic virus (CaMV) (Odell et al., Nature 313:810-812 (1985)), promoters of Chlorella virus methyltransferase genes (U.S. Pat. No. 5,563,328), and the full-length transcript promoter from figwort mosaic virus (FMV) (U.S. Pat. No. 5,378,619); the promoters from such genes as rice actin (McElroy et al., Plant Cell 2:163-171 (1990)), ubiquitin (Christensen et al., Plant Mol. Biol. 12:619-632 (1989) and Christensen et al., Plant Mol. Biol. 18:675-689 (1992)), pEMU (Last et al., Theor. Appl. Genet. 81:581-588 (1991)), MAS (Velten et al., EMBO J. 3:2723-2730 (1984)), maize H3 histone (Lepetit et al., Mol. Gen. Genet. 231:276-285 (1992) and Atanassova et al., Plant Journal 2(3):291-300 (1992)), Brassica napus ALS3 (PCT application WO 97/41228); and promoters of various Agrobacterium genes (see U.S. Pat. Nos. 4,771,002, 5,102,796, 5,182,200 and 5,428,147). Others are also known to the art, including The promoter can be any plant-expressible promoter of any of a number of known plant disease and herbivore resistance genes, genes of soybean cv. Williams 82, promoters of ubiquitin, MADS domain transcription factor, cellulase, polygalacturonase, nopaline synthase (NOS), octopine synthase (OCS), mannopine synthase (MAS), cauliflower mosaic virus 19S and 35S (CaMV19S, CaMV35S), enhanced CaMV (eCaMV), ribulose 1,5-bisphosphate carboxylase (ssRUBISCO), figwort mosaic virus (FMV), CaMV derived AS4, tobacco RB7, wheat POX1, tobacco EIF-4, lectin protein (Lel), or rice RC2.

Finally, promoters composed of portions of other promoters and partially or totally synthetic promoters can be used. See, e.g., Ni et al., Plant J., 7:661-676 (1995) and POT WO 95/14098 describing such promoters for use in plants.

The promoter can include, or be modified to include, one or more enhancer elements. Preferably, the promoter will include a plurality of enhancer elements. Promoters containing enhancer elements provide for higher levels of transcription as compared to promoters that do not include them. Suitable enhancer elements for use in plants include the PCISV enhancer element (U.S. Pat. No. 5,850,019), the CaMV 35S enhancer element (U.S. Pat. Nos. 5,106,739 and 5,164,316) and the FMV enhancer element (Maiti et al., Transgenic Res., 6, 143-156 (1997)). See also PCT WO 96/23898 and Enhancers And Eukaryotic Expression (Cold Spring Harbor Press; Cold Spring Harbor, N.Y., 1983).

A 5′ untranslated sequence is also employed adjacent to the end of the coding sequence. The 5′ untranslated sequence is the portion of an mRNA which extends from the 5′ CAP site to the translation initiation codon. This region of the mRNA is useful for translation initiation in plants and plays a role in the regulation of gene expression. Suitable 5′ untranslated regions for use in plants include those of alfalfa mosaic virus, cucumber mosaic virus coat protein gene, and tobacco mosaic virus, among others.

For efficient expression, the coding sequences are preferably also operatively linked to a 3′ untranslated sequence. The 3′ untranslated sequence will include a transcription termination sequence and a polyadenylation sequence. The 3′ untranslated region can be obtained from the flanking regions of genes from Agrobacterium, plant viruses, plants or other eukaryotes. Suitable 3′ untranslated sequences for use in plants include those of the cauliflower mosaic virus 35S gene, the phaseolin seed storage protein gene, the pea ribulose biphosphate carboxylase small subunit E9 gene, the soybean 7S storage protein genes, the octopine synthase gene, mannopine synthase gene and the nopaline synthase gene.

As used herein, a “vector” is a composition which can transduce, transform or infect a cell, thereby causing the cell to express nucleic acids carried by the vector, and, optionally, proteins other than those native to the cell, or in a manner not native to the cell. A vector includes a nucleic acid (ordinarily RNA or DNA) to be expressed by the cell (a “vector nucleic acid”). A vector optionally includes materials to aid in achieving entry of the nucleic acid into the cell, such as a retroviral particle, liposome, protein coating or the like. The vector and/or other construct can also include, within the coding region of interest, a nucleic acid sequence that acts, in whole or in part, to terminate transcription of that region. For example, such termination sequences include the Tr7 3′ sequence and the nos 3′ sequence (Ingelbrecht et al., The Plant Cell 1:671680, 1989; Bevan et al., Nucleic Acids Res. 11:369 385, 1983) and the like. The vector and/or other construct can also include regulatory elements. Examples of such regulatory elements include the Adh intron 1 (Callis et al., Genes and Develop. 1:1183 1200, 1987), the sucrose synthase intron (Vasil et al., Plant Physiol. 91:1575 1579, 1989), and the TMV omega element (Gallie et al., The Plant Cell 1:301311, 1989). The vector and/or other construct can also include a selectable marker, a screenable marker and/or other elements as appropriate. Examples of these elements and markers mentioned herein are known in the art and can be readily used without undue experimentation in the methods and constructs described herein. The vector can contain one or more replication systems which allow it to replicate in host cells. Self-replicating vectors include plasmids, artificial chromosomes, cosmids and viral vectors. Alternatively, the vector can be an integrating vector which allows the integration into the host cell's chromosome of heterologpis DNA sequences. The vector desirably also has unique restriction sites for the insertion of DNA sequences. If a vector does not have unique restriction sites, it can be modified to introduce or eliminate restriction sites to make it more suitable for further manipulations.

As used herein “conservative amino acid substitutions” are those that result in variants and equivalents that retain their functionality, for example, the substitution of one or more amino acids by similar amino acids, e.g., the substitution of an amino acid within the same general class, such as an acidic amino acid, a basic amino acid, or a neutral amino acid, by another amino acid within the same class.

As used herein, “probe” means an oligonucleotide or short fragment of DNA designed to be sufficiently complementary to a sequence in a denatured nucleic acid to be probed and to be bound under selected stringency conditions.

As used herein, a “stringent condition” is functionally defined with regard to hybridization of a nucleic-acid probe to a target nucleic acid (i.e., to a particular nucleic acid sequence of interest) by the specific hybridization procedure discussed in Sambrook et al. (Molecular Cloning: A Laboratory Manual, Cold Spring Harbor Laboratory Press, Cold Spring Hobart, 1989, at 9.52 9.55). Regarding the amplification of a target nucleic acid sequence (e.g., by PCR) using a particular amplification primer pair, “stringent conditions” are conditions that permit the primer pair to hybridize substantially only to the target nucleic acid sequence to which a primer having the corresponding wild-type sequence (or its complement) would bind so as to produce a unique amplification product. For hybridization of a probe or primer from one plant species to a polynucleotide of another plant species in order to identify homologs, preferred hybridization and washing conditions are as discussed in Sambrook et al (supra, at 9.47 9.57, wherein “highly stringent conditions” include hybridization at 65° C. in a hybridization solution that includes 6 times SSC and washing for 1 hour at 65° C. in a wash solution that includes 0.5 times SSC, 0.5% SOS. “Moderate stringency” conditions are similar except that the temperature for the hybridization and washing steps is a lower temperature at which the probe is specific for a target sequence, such as at least 42° C., or at least 50° C., or at least 55° C., or at least 60° C. Alternatively, “highly-stringent conditions can include hybridization under conditions comprising a buffer comprising 50% formamide at about 37° C. to 42° C.; or, 42° C. in 50% formamide, 5× SSPE, 0.3% SDS, and a wash step comprising use of a buffer comprising 0.15 NaCl for 15 min at 72° C.

Two nucleic acid sequences are “genetically linked” when the sequences are in linkage disequilibrium.

As used herein, a “tissue sample” is any sample that comprises more than one cell. In a preferred aspect, a tissue sample comprises cells that share a common characteristic (e.g., derived from a leaf, root, or pollen, or from an abscission layer, etc.).

As used herein, a “3′ untranslated region” or “3′ untranslated nucleic acid sequence” or “3′ transcriptional termination signal” refers to the 3′ end of a piece of transcribed but untranslated nucleic acid sequence that functions in a plant cell to cause transcriptional termination and/or the addition of polyadenylate nucleotides to the 3′ end of the RNA sequence being produced. Typically, a DNA sequence located from four to a few hundred base pairs downstream of the polyadenylation site serves to terminate transcription. The region is required for efficient polyadenylation of transcribed messenger RNA (mRNA). RNA polymerase transcribes a coding DNA sequence through a site where polyadenylation occurs.

As used herein, “transformation” refers to the transfer of a nucleic acid sequence into the genome of a host organism such as a host plant, resulting in genetically stable inheritance. Transformed host plants containing the nucleic acid sequences are referred to as “transgenic plants.”

The term “is associated with” as used herein in the context of the Aphis glycines resistance trait being “associated with” a marker, means that the trait locus has been found, using marker-assisted analysis, to be present in soybean plants showing Aphis glycines resistance in live bioassays as described herein.

The term, “modification of the nucleic acid sequencer” refers to modification of a nucleic acid sequence, such as the Rag2 nucleic acid sequence described herein, by techniques such as site-directed mutagenesis. Such techniques allow one or more of the amino acids encoded by a nucleic acid molecule to be altered (e.g. a Cysteine to be replaced by a Tyrosine). Specific techniques include cassette mutagenesis (Wells et al., Gene 34:315 23, 1985), primer extension (Gilliam et al., Gene 12:129 137, 1980; Zoller and Smith, Methods Enzymol. 100:468 500, 1983; Dalbadie-McFarland et al. Proc. Natl. Acad. Sci. (U.S.A.) 79:6409 6413, 1982) and methods based upon PCR (Scharf et al., Science 233:1076 1078, 1986; Higuchi et al., Nucleic Acids Res. 16:7351 7367, 1988). Site-directed mutagenesis strategies have been applied to plants in vitro as well as in vivo.

An “allele” is any of one or more alternative forms of a gene, all of which alleles relate to one trait or characteristic. In a diploid cell or organism, the two alleles of a given gene occupy corresponding loci on a pair of homologous chromosomes. The Rag1 and Rag2 genes can be allelic to each other.

“Germplasm” means the genetic material with its specific molecular and chemical makeup that comprises the physical foundation of the hereditary qualities of an organism. As used herein, germplasm includes seeds and living tissue from which new plants can be grown; or, another plant part, such as leaf, stem, pollen, or cells, that can be cultured into a whole plant. Germplasm resources provide sources of genetic traits used by plant breeders to improve commercial cultivars.

“Hybrid plant” means a plant offspring produced by crossing two genetically dissimilar parent plants.

“Inbred plant” means a member of an inbred plant strain that has been highly inbred so that all members of the strain are nearly genetically identical.

“Introgression” means the entry or introduction by hybridization of a gene or trait locus from the genome of one plant into the genome of another plant that lacks such gene or trait locus.

“Molecular marker” is a term used to denote a nucleic acid or amino acid sequence that is sufficiently unique to characterize a specific locus on the genome. Examples include restriction fragment length polymorphisms (RFLPs) and single sequence repeats (SSRs). RFLP markers occur because any sequence change in DNA, including a single base change, insertion, deletion or inversion, can result in loss (or gain) of a restriction endonuclease recognition site. The size and number of fragments generated by one such enzyme is therefore altered. A probe that hybridizes specifically to DNA in the region of such an alteration can be used to rapidly and specifically identify a region of DNA that displays allelic variation between two plant varieties. SSR markers occur where a short sequence displays allelic variation in the number of repeats of that sequence. Sequences flanking the repeated sequence can serve as polymerase chain reaction (PCR) primers. Depending on the number of repeats at a given allele of the locus, the length of the DNA segment generated by PCR will be different in different alleles. The differences in PCR-generated fragment size can be detected by gel electrophoresis. Other types of molecular markers are known. All are used to define a specific locus on the soybean genome. Large numbers of these have been mapped. Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming, requiring growing up of plants to a stage where the trait can be expressed.

“Linkage” is defined by classical genetics to describe the relationship of traits that co-segregate through a number of generations of crosses. Genetic recombination occurs with an assumed random frequency over the entire genome. Genetic maps are constructed by measuring the frequency of recombination between pairs of traits or markers. The closer the traits or markers lie to each other on the chromosome, the lower the frequency of recombination, the greater the degree of linkage. Traits or markers are considered herein to be linked if they generally co-segregate. A 1/100 probability of recombination per generation is defined as a map distance of 1.0 centiMorgan (10 cM). Preferably markers useful for screening for the presence of Aphis glycines resistance map to within about 20 cM of the trait, or within about 10 cM of the trait or within about 5 cM of the trait. A second marker that maps to within about 10 cM of a first marker that co-segregates with the Rag2 trait and generally co-segregates with the Rag2 trait is considered equivalent to the first marker. Any marker that maps within 10 cM or 5 cM of the Rag2 trait belongs to the class of preferred markers for use in screening and selection of soybean germplasm having the Rag2 Aphis glycines resistance trait. A number of markers are known to the art to chromosome 13 on which the Rag2 gene is found. A number of markers are proprietary markers known only to certain of those skilled in the art of soybean plant breeding. A proprietary marker mapping within about 10 cM, or about 5 cM, of any publicly known-marker specified herein is considered equivalent to that publicly-known marker.

“Linkage group” refers to traits or markers that generally co-segregate. A linkage group generally corresponds to a chromosomal region containing genetic material that encodes the traits or markers.

“Rag2 resistance” or “Rag2-derived resistance” means resistance in a soybean germplasm to Aphis glycines that is provided by the heterozygous or homozygous expression of the Rag2 gene by soybean germplasm, as demonstrated by resistance to Aphis glycines after inoculation with same according to the methods described herein.

“Self-crossing or self-pollination” is a process through which a breeder crosses hybrid progeny with itself, for example, a second generation hybrid F2 with itself to yield progeny designated F2:3.

“Locus” means a chromosomal region where a polymorphic nucleic acid or trait determinant or gene is located.

As used herein, “regeneration” refers to the process of growing a plant from a plant cell or tissue (e.g., plant protoplast or explant). The regeneration, development, and cultivation of plants such as soybean plants from transformants or from various transformed explants containing a foreign, exogenous gene that encodes a protein of interest are well known in the art (Weissbach and Weissbach, In: Methods for Plant Molecular Biology, Eds, Academic Press, Inc. San Diego, Calif., 1988). This regeneration and growth process can include the steps of selection of transformed cells containing exogenous Rag2 genes and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

A “modified Rag2 nucleic acid molecule” is used herein to describe embodiments in which the Rag2 nucleic acid molecules are modified by site-directed mutagenesis strategies or other means known to the art. They can be used to confer or contribute to conferring aphid resistance to plants lacking such resistance, or they can be used as nucleic acid molecules to target other nucleic acid molecules, e.g., for further modification. The Rag2 protein that is encoded by the modified Rag2 nucleic acid is referred to as a “modified Rag2 protein.” It is understood that mutants with more than one altered nucleotide can be constructed using techniques that practitioners skilled in the art are familiar with such as isolating restriction fragments and ligating such fragments into an expression vector (see, for example, Sambrook et al., Molecular Cloning: A Laboratory Manual, Cold Spring Harbor Press, 1989). Modified Rag2 nucleic acids and amino acids that can confer or contribute to conferring Rag2 resistance on a plant are equivalents of the Rag2 nucleic acids specifically described herein, and are included within the scope of the claims hereof. The coding sequence of the Rag2 gene hereof can be extensively altered, for example, by fusing part of it to the coding sequence of a different gene to produce a novel hybrid gene that encodes a fusion protein or chimeric protein. A chimeric protein can be made by a conventional method available in the art, see, for example, Sambrook et al., Molecular Cloning: A Laboratory Manual, Cold Spring Harbor Press, 1989. A chimeric protein hereof can be made by combining any two available aphid-resistance nucleic acid sequences that encode aphid resistance proteins. In one example hereof, the chimeric protein can be produced by fusing all or part of the soybean Rag2 nucleic acid sequence that encodes C-terminal portion of the soybean Rag2 protein to all or parts of nucleic acid sequences that encode aphid resistance in other plants or all or parts of nucleic acid sequences encoding other aphid resistance proteins in soybean (see, e.g., Hill, C. B. et al, “Inheritance of Resistance to the Soybean Aphid in Soybean PI 200538,” Crop Sci. 49:1193-1200 (2009)). All such constructs are included within the scope of the claims hereof.

The following examples further demonstrate several preferred embodiments hereof. Those skilled in the art will recognize numerous equivalents to the specific embodiments described herein. Such equivalents are intended to be within the scope hereof. Although methods and materials similar or equivalent to those described herein can be used in the practices or testing described herein, suitable methods and materials are described below. Standard recombinant DNA and molecular cloning techniques used herein are well known in the art and/or described herein.

### Examples

### Fine Mapping of the Soybean Aphid Resistance Gene Rag2 in Soybean PI 200538

The discovery of biotype diversity of soybean aphid [SA: Aphis glycines Matsumura] in North America emphasizes the necessity to identify new aphid resistance genes. The soybean [Glycine max (L.) Merr.] plant introduction (PI) 200538 is a promising source of soybean aphid resistance because it shows a high level of resistance to a soybean aphid biotype that can overcome the soybean aphid resistance gene Rag1 from soybean variety Dowling (PI 548663). The soybean aphid resistance gene Rag2 was previously mapped using soybean accession No. PI 200538 to a 10 centiMorgan (cM) marker interval on soybean chromosome 13 (formerly linkage group F (LG F). To fine map Rag2, high resolution linkage analysis was carried out using lines derived from 6,160 F2 plants at different levels of backcrossing that were screened with flanking genetic markers for the presence of recombination in the Rag2 interval. Fifteen single nucleotide polymorphism (SNP) markers and two dominant polymerase chain reaction (PCR) based markers near Rag2 were developed by re-sequencing target intervals and sequence tagged sites (STSs). These efforts resulted in the mapping of Rag2 to a 54-kilobase (kb) interval on the Williams 82 8× assembly (Glyma1). This Williams 82 interval contains seven predicted genes including one nucleotide binding site-leucine rich repeat (NBS-LRR) gene. SNP marker and gene information identified in this study are an important resource in marker-assisted selection (MAS) for aphid resistance and for cloning the gene.

### EXAMPLES

Previous research mapped Rag2 to a 10-cM interval from PI 200538 and a 4.5-cM interval from PI 243540 (Hill et al. 2009; Mian et al. 2008b) resulting in large gaps between the gene and markers on these linkage maps. The objective of this study was to fine map the location of Rag2 from PI 200538 through the identification of additional genetic recombinants close to the gene and the development of single nucleotide polymorphism (SNP) markers by re-sequencing sequence-tagged sites (STSs) and target regions based on the Williams 82 89 draft assembly (Glyma1) (Schmutz et al. 2010). Fine mapping and high resolution linkage analysis of the region containing Rag2 facilitateS soybean aphid resistance breeding because SNP markers developed during this process that are closely linked to or within Rag2 can be used for marker-assisted selection (MAS).

### Materials and Methods

### Plant Material

To fine map Rag2, three sources of soybean populations were used. The first was a population of 95 F_{2:3} lines from the three-way cross LD02-4485 9 (Ina 9 PI 200538) that was originally used to genetically map Rag2 (Hill et al. 2009). The population was phenotyped for aphid resistance and genotyped with SSR markers in the F₂ and F_{2:3} generations as described by Hill et al. (2009). PI 200538 (Sugao Zairai) is a maturity group (MG) VIII soybean accession originating from Japan (USDA-ARS germplasm Resources Information Network, http://www.ars-grin.gov/npgs/; accessed 26 Oct. 2009). Ina is a MG IV (relative maturity 4.5) soybean cyst nematode (SCN) (Heterodera glycines Ichinohe)-resistant cultivar that is susceptible to SA (Nickell et al. 1999; Hill et al. 2004a). LD02-4485 is a high yielding MG II experimental line developed by the University of Illinois that is SA susceptible and SCN resistant.

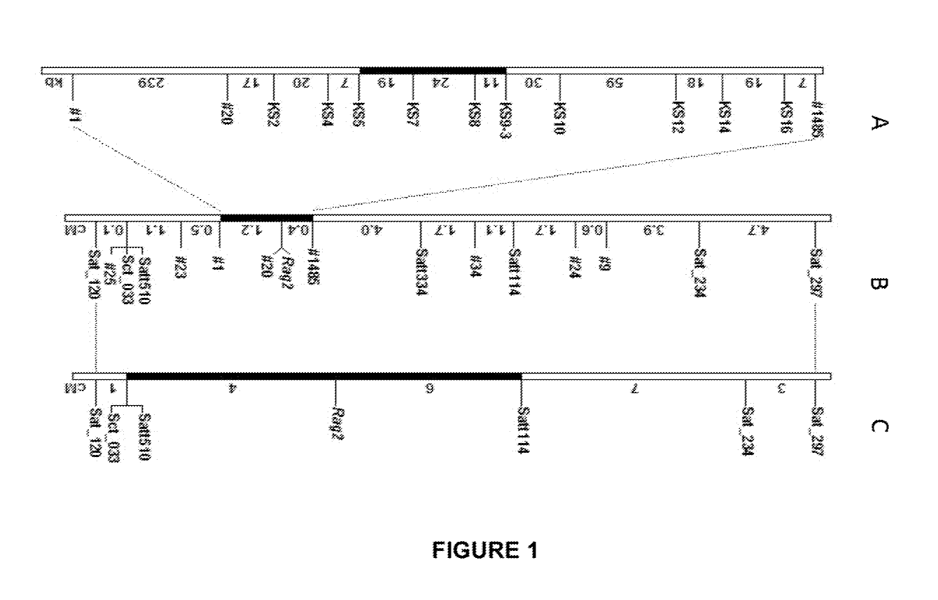

The second source of germplasm used in the Rag2 fine mapping was a set of BC₂F_{2:3} BC₃F_{2:3}, and F_{2:3} lines derived from plants selected for having putative genetic recombination in the interval containing Rag2 (Table 1). These were selected from a total of 3,151 BC₂F₂, BC₃F₂, and F₂ plants segregating for Rag2 that were grown in the field at Urbana. These plants were screened with the SSR markers Satt510 and Satt114 (Song et al. 2004), which flanked the gene (Hill et al. 2009). One hundred and eighty-five plants with recombination events between the markers were selected and harvested. These selected recombinant lines were tested with three SNP markers (#1485, #20, and #1) that map close to the Rag2 region to identify which lines have recombination events near the gene (Tables 2, 3; FIG. 1b). From the marker screening, 12 lines with recombination events in the marker intervals were identified and five were selected for SA resistance testing. Selected lines 18 and 32 are BC₂F_{2:3} lines with the pedigree LD03-6566 (3) 9 [LD02-4485 9 (Ina 9 PI 200538)] (Table 1). Line 86 is a F_{2:3} line with the pedigree L005-3230 9 [LD02-4485 (3) 9 (Ina 9 PI 200538)]. Lines 162 and 181 are BC3F2:3 lines that both have the pedigree LD02-4485 (4) 9 (Ina 9 PI 200538). These last two lines were also tested with all SNP markers in the region except KS9-3 (Table 2). Both LD05-3230 and LD03-6566 are high yielding, SA susceptible experimental lines developed by the University of Illinois soybean breeding program.

The third source of germplasm was a set of 58 lines (K1-K58) selected for having putative genetic recombination in the interval containing Rag2 (Table 1) from a total of 2,632 BC3F2, F2, and BC4F2 plants segregating for Rag2 that were grown in the field at Urbana, Ill. These plants were first screened with the SNP markers #20 and #1485, which flanked the gene (Table 2; FIG. 1b). Fifty-eight plants with recombination between the markers were selected and screened with SNP markers KS7 and KS12 to identify which plants had recombination events close to the gene (Table 2).

From the marker screening, three plants with recombination events between KS7 and KS12 were identified, and lines were derived from the three plants and used in progeny tests. Selected line K16 is a BC3F2:3 line with the pedigree L003-6566 (4) 9 [LD02-4485 9 (Ina 9 PI 200538)]. Lines K31 and K37 are in the F2:3 generation and have the pedigree LD03-6566 9 [L002-4485 (4) 9 (Ina 9 PI 200538)]. Progeny plants from the three recombinant lines were tested with all SNP markers.

TABLE 1
Pedigree information for 243 recombinant lines identified
during the fine mapping of Rag2 from PI 200538

| | No. of tested | | |
|---|---|---|---|
| | recombinant | | Generation |
| Line No. | lines | Pedigree of Lines | of line |
| 1-8 | 8 | LD02-8782 × LD02-4485 (3) × | F_{2:3} |
| | | (Ina × PI 200538) | |
| 9-36 | 28 | LD03-6566 (3) × [LD02-4485 × | BC₂ F_{2:3} |
| | | (Ina × PI 200538)] | |
| 37-52 | 16 | LD04-8782 × LD03-6566 (2) × | F_{2:3} |
| | | [LD02-4485 × (Ina × | |
| | | PI 200538)] | |
| 53-103 | 51 | LD05-3230 × [LD02-4485 (3) × | F_{2:3} |
| | | (Ina × PI 200538)] | |
| 104-156 | 53 | LD05-3230 × LD03-6566 (2) × | F_{2:3} |
| | | [LD02-4485 × (Ina × | |
| | | PI 200538)] | |
| 157-185 | 29 | LD02-4485 (4) × (Ina × | BC₃ F_{2:3} |
| | | PI 200538) | |
| K1-K29 | 29 | LD03-6566 (4) × [LD02-4485 × | BC₃ F_{2:3} |
| | | (Ina × PI 200538)] | |
| K30-K48 | 19 | LD03-6566 × [LD02-4485 (4) × | F_{2:3} |
| | | (Ina × PI 200538)] | |
| K49-K58 | 10 | LD02-4485 (5) × (Ina × | BC₄ F_{2:3} |
| | | PI 200538) | |

TABLE 2
SNP marker genotypes of eight recombinant lines and their reaction to aphid biotype 2

| Physical | | | | | | | | | | | | | | |
|---|---|---|---|---|---|---|---|---|---|---|---|---|---|---|
| position | | | | | | | | | | | | | | |
| (Mb)^{a} | | 29.097 | 29.104 | 29.128 | 29.141 | 29.182 | 29.212 | 29.223 | 29.247 | 29.266 | 29.273 | 29.293 | 29.310 | 29.549 |
| | Pheno- | | | | | | | | | | | | | |
| Line | type^{f} | #1485 | KS16 | KS14 | KS12 | KS10 | KS9-3 | KS8 | KS7 | KS5 | KS4 | KS2 | #20 | #1 |
| 18 | Segre- | H^{g} | NT | NT | NT | NT | NT | NT | NT | NT | NT | NT | H | S |
| | gation | | | | | | | | | | | | | |
| 32 | Resist- | R | NT | NT | NT | NT | NT | NT | NT | NT | NT | NT | R | H |
| | ance | | | | | | | | | | | | | |
| 86 | Segre- | H | NT | NT | NT | NT | NT | NT | NT | NT | NT | NT | H | R |
| | gation | | | | | | | | | | | | | |
| 162 | Resist- | R | R | R | R | R | NT | R | R | H | H | H | H | H |
| | ance | | | | | | | | | | | | | |
| 181 | Segre- | R | R | R | H | H | NT | H | H | H | H | H | H | H |
| | gation | | | | | | | | | | | | | |
| K37 | Segre- | S | S | S | S | S | H | H | H | H | H | H | H | H |
| | gation | | | | | | | | | | | | | |
| K16 | Resist- | H | H | H | H | H | H | R | R | R | R | R | R | R |
| | ance | | | | | | | | | | | | | |
| K31 | Resist- | H | H | H | H | H | H | R | R | R | R | R | R | R |
| | ance | | | | | | | | | | | | | |

| | | Marker used | | No. of | Aphid numbers^{c} | | | | |
|---|---|---|---|---|---|---|---|---|---|
| Line | Phenotype^{f} | in F test | Test No.^{b} | plants tested | R | H | S | P > F^{d} | R^{3e} |
| 18 | Segregation | #20 | 1 | 42 | 24 | 22 | 114 | <0.001 | 0.91 |
| 32 | Resistance | #1 | 1 | 43 | 23 | 25 | 29 | 0.22 | 0.07 |
| 86 | Segregation | #20 | 2 | 38 | 349 | 337 | 2774 | <0.001 | 0.97 |
| 162 | Resistance | KS5 | 2 | 67 | 74 | 104 | 93 | 0.44 | 0.02 |
| 181 | Segregation | KS12 | 2 | 66 | 107 | 151 | 2252 | <0.001 | 0.93 |
| K37 | Segregation | KS9-3 | 3 | 40 | 54 | 72 | 1088 | <0.001 | 0.99 |
| K16 | Resistance | KS9-3 | 3 | 41 | 61 | 55 | 46 | 0.41 | 0.04 |
| K31 | Resistance | KS9-3 | 3 | 40 | 22 | 24 | 23 | 0.86 | 0.01 |

^{a}Physical position of the markers based on the Williams 82 8x assembly (Glyma1) available at http://www.phytozome.net (Schmutz et al. 2010). The mega base (Mb) positions of the SNP markers correspond to the locations of each SNF and the positions of the dominant markers are the locations of the end sequences of the reverse primers
^{b}SA resistance test. Test 1 was conducted in a plant growth chamber maintained at 22-25° C. with 14 h illumination with 30 μmol m⁻²s⁻¹ PAR irradiation. Test 2 was conducted in a plant growth chamber maintained at 22-25° C. with 14 h illumination with 300 μmol m⁻²s⁻¹ PAR irradiation, and Test 3 was conducted in a greenhouse maintained at 22-25° C. with 14 h illumination with 1,000 W high pressure sodium vapor lights.
^{c}Mean number of aphids on each plant predicted to be homozygous resistant (R), heterozygous (H), and homozygous susceptible (S) for Rag2 based on the segregation of the marker listed three columns to the left
^{d}Significance level of the marker association
^{e}R³ value of the marker association
^{f}Phenotype of the line based on aphid numbers and the marker association test
^{g}Marker genotypes of the recombinant plants that formed the recombinant lines; R homozygous for the allele from Pt 200548, H heterozygous, S homozygous for the allele from the susceptible percent
NT not tested. Italicized letters are placed at the genetic interval containing inferred recombination event.

### Aphid Culture

The SA biotype 2 was established at the Ohio Agricultural Research and Development Center (OARDC), Wooster, Ohio during the summer of 2005 by collecting aphids from nearby soybean fields. Biotype 2 was maintained on a continuous supply of plants of L005-1 6611, an experimental line developed by the University of Illinois, which has Rag1 from Dowling. The SAs were maintained in a growth chamber at 22° C. and under 16-h irradiation and 70% relative humidity (Hill et al. 2004a).

### Soybean Aphid Biotype 2 Resistance Evaluation

The 95 F2:3 lines from the LD02-4485 9 (Ina 9 PI 200538) population were tested for resistance to SA biotype 2 as described by Hill et al. (2009). Briefly, a minimum of 11 F3 plants from each line were tested with SA biotype 2 in a greenhouse and rated for aphid colonization on a 1-4 scale with 1=few solitary live aphids and 4=dense colonies accompanied by plant damage. The greenhouse was maintained at 22-25° C. with 14 h ambient light supplemented by 1,000-W high pressure sodium vapor lamps positioned approximately 2 m above the greenhouse benches (Hill et al. 2004a). Progeny from the eight selected recombinant plants were tested for SA resistance using choice tests with biotype 2 in order to determine the position of the Rag2 gene relative to the recombination points in each line. Lines 18 and 32 were the first evaluated for resistance. This was followed by testing lines 86, 162, and 181, followed by testing of K16, K31, and K37. These tests included from 38 to 67 progeny plants from each selected recombinant line (Table 2). In all SA resistance tests, the photoperiod was 14 h and the temperature was between 22 and 25° C. The first SA resistance test was conducted in a plant growth chamber with 30 μmol m⁻²s⁻¹ photosynthetically active radiation (PAR) (Kim et al. 2008). The second SA resistance test was conducted in plant growth chamber with 300 μmol m⁻²s⁻¹ PAR. The third SA resistance test was conducted in a greenhouse under conditions described above.

Individual plants were grown in 60 by 60 by 60-mm plastic 48-pot inserts (Hummert Intl., Earth city. MO, USA) contained inside plastic trays without holes (Hummert Intl.) as described by Kim et al. (2008). Each 48-pot insert included 44 progeny from a line and two replications of the parents PI 200538 and LD02-4485. The 48 plants in an insert were arranged in a completely randomized design. Experiments were inoculated by placing leaves of LD05-16611 that were infested with 200-300 aphids of biotype 2 at all life stages on top of VE-stage seedlings. Ten days after inoculation, resistance was evaluated by counting the total number of aphids on each plant.

### DNA Extraction

Genomic DNA from the 95 F2 plants was extracted using young trifoliolate leaves of each plant with the CTAB (hexadecylatrimethylammonium bromide) method described by Honeycutt et al. (1992) with minor modifications (Hill et al. 2009). Because tissue samples were collected during the SA resistance evaluation experiment, aphids were eliminated from leaves with the systemic insecticide imidacloprid prior to sampling.

Genomic DNA from the 3,151 BC3F2, BC2F2, and F2 plants in 2008 and 2,632 BC3F2, F2, and BC4F2 plants was extracted using young trifoliolate leaf tissue from each plant using the quick extraction method (Bell-Johnson et al. 1998). Genomic DNA from 185 recombinant lines selected in earlier and 58 recombinant plants selected in the same year was extracted using young trifoliolate leaf tissue pooled from 12 progeny plants from each line using the CTAB method described above.

After the completion of the resistance assays, genomic DNA from each of the 377 individual progeny from the eight selected recombinant lines in these assays was extracted using the STAB method. All CTAB DNA was quantified by ND-1000 Spectrophotometer (NanoDrop Technologies, Wilmington, Del., USA) and diluted to 25 ng μl⁻¹ for SSR genotyping and 20 ng μl⁻¹ for SNP genotyping.

SSR Marker Re-Screening within the Interval Containing Rag2

All SSR markers previously mapped between Satt510 and Satt114 on chromosome 13 (LG F) were retested to determine if any were polymorphic between Pt 200538 and LD02-4485, the two parents of the first mapping population that includes 95 F2:3 lines. The primer sequences and location of the SSR markers are available from Soybean Linkage Map-2006 (http://bfgl.anri.barc.usda.gov/cgi-bin/soybean/Linkage.pl; accessed 27 May 2009). Polymerase chain reaction (PCR) was performed according to Cregan and Quigley (1997). PCR reactions consisted of 36 cycles of denaturation at 94° C. for 25 s, annealing at 46° C. for 25 s, and extension at 68° C. for 25 s with a PTC 100 Programmable Thermal Controller (MJ Research Inc., Watertown, Mass., USA) with slight modifications according to the specific annealing temperature of the primers. The PCR products were first analyzed in 3% agarose gels (BMA, Rockland, Me., USA) and then retested in 3% metaphor-agarose gels with ethidium bromide staining in 19 Tris-Borate-EDTA buffer.

### Re-Sequencing of STSs

To develop additional markers and narrow the gene interval, re-sequencing of STSs already mapped to the region on soybean chromosome 13 (LG F) where Rag2 is located was performed. Information on target amplification primer pairs for the 34 STSs (Table 3) containing SNPs from this region was obtained from Hyten et al. (2010). These STSs were first re-sequenced to determine if the SNPs previously identified were present between the parents of the recombinant lines. A target amplification primer pair for one additional STS, GF097621 (Table 3), was designed using IDT SciTools PrimerQuestSM software (Integrated DNA Technologies, Coralville, Iowa, USA). The re-sequencing was done by first PCR amplifying the STSs.

Amplification reactions were conducted with 100 ng of parental DNA, 0.25 IM of forward and reverse primer, 19 of buffer (BioLabs Inc., MA, USA), 0.25 mM of each dNTP (Applied Biosystems, Foster City, Calif., USA), and 1 U of Taq polymerase (BioLabs Inc.), in a total volume of 40 II. The reaction mixture was denatured at 95° C. for 1 min and subjected to 28 cycles of 94° C. for 30 s, annealing at 55° C. for 40 s, and extension at 68° C. for 2 min 20 s, followed by one cycle of 8 min at 68° C. using a PTC 100 Programmable Thermal Controller (MJ Research Inc.).

PCR products were resolved by gel electrophoresis in 0.9% TAE agarose gels stained with ethidium bromide. The presence of a single PCR product was verified for each primer pair. If primer pairs produced no product or multiple products, annealing temperatures or PCR cycles were modified to identify the optimum PCR condition for each primer pair (Choi et al. 2007). When single PCR products from the two parents were produced, these were purified with the QIAquick Gel Extraction Kit (Qiagen, Calif., USA). Purified PCR products were then sequenced from both ends using the same primers as for PCR amplification with the ABI BigDye Terminator v3.1 cycle sequencing kit on an ABI PRISM 3730 sequencer (Applied Biosystems) at the University of Illinois Keck Center Core Facility. To detect SNPs between two parents, ABI trace files were analyzed by Sequencher version 4.9 (Gene Codes Corporation, Ann Arbor, Mich., USA).

Saturation of the Rag2 Region with Additional SNP Markers

Once Rag2 was positioned relative to the SNPs developed from the STSs, direct re-sequencing of target regions was conducted to develop additional SNP markers that could be used to better define the genomic position of Rag2. A total of 27 target amplification primer pairs were designed every 10 kb between nucleotides 29,097 and 29,310 kb on chromosome 13 based on the Williams 82 89 draft assembly (Glyma1) (Schmutz et al. 2010). The primers were designed with IDT SciTools PrimerQuestSM software (Integrated DNA Technologies). The uniqueness of each primer pair in the genome was double-checked by BLAST analysis. Sequencing and SNP detection were conducted as described above.

### SNP Marker and Dominant Marker Genotyping

Target amplification primers and probes for TaqMan assays or melting curve assays (MCAs) were designed for the confirmed SNPs. Target amplification primers and simple probe for MCA were designed by use of the LightCycler-Probe Design Software 2.0 (Roche Diagnostics, Switzerland) and were blasted to Williams 82 89 draft assembly (Glyma1)) to check whether there was a single match of the sequences and to verify their position in the soybean genome.

If a target amplification primer or simple probe matched multiple regions of the soybean genome, the primer or probe was redesigned until it had a single match in the soybean genome. TaqMan primers and probes were designed by Assays-by-Design Service (Applied Biosystems). SNP marker genotyping using TaqMan assays or MCAs was conducted with the Roche LightCycler_{—}480 System (Roche Diagnostics, Indianapolis, Ind., USA) described by Kaczorowski et al. (2008).

The target sequencing primer pairs KS8 and KS10 produced a desired single PCR product only for the susceptible parent LD02-4485 and were used as dominant markers. These markers could distinguish homozygous resistance alleles from homozygous susceptible alleles, but could not distinguish homozygous susceptible alleles from heterozygous alleles. Therefore, plants having no PCR product were classified as homozygous resistance and those with a product were classified as heterozygous or homozygous susceptible. The forward and reverse primer sequences of KS8 were 5′-TACCCTCAAATGGACTTGGTGCCT-3′ [SEQ ID NO:1]; and 5-GGCGATGGTGATCTTGACTGTCT-3′ [SEQ ID NO:2], receptively. The forward and reverse primer sequences of KS10 were 5′-TCCCATTACGCCGTTCAGCAAGAT-3′ [SEQ ID NO:3] and 5′-GGTGTACAAGGAAAGCCCAAGACT-3[SEQ ID NO:4]. PCR condition for these markers was the same as for the SSR marker and the PCR products were analyzed in 1% agarose gels. All markers for lines with genotypes given on Table 2 were tested on each progeny plant in the aphid assays resulting in 3-13 markers tested on these progeny plants.

### Genetic Mapping and Statistical Analysis

Molecular marker and phenotype data were used to construct a genetic map with the 95 F2:3 lines from the L002-4485 9 (Ina 9 PI 200538) population. Linkage analysis was performed with JoinMap 3.0 (Van Ooijen and Voorrips 2001) using the Kosambi mapping function. A logarithm (base 10) of the odds (LOD) score of 5.0 was used as a threshold to group markers into a linkage group.

To test whether Rag2 was segregating among plants in each of the eight recombinant lines phenotyped for SA resistance, the progeny plants that had been evaluated for SA resistance were tested with a polymorphic marker in each line that maps near Rag2. Single factor analysis of variance was used to identify the associations between SA resistance and marker segregation using the PROC GLM procedure of SAS (SAS Institute 2002).

### Results

### Genetic Mapping of Rag2 Using SSR Markers and SNP Markers Developed by STS Re-Sequencing

The retesting of all SSR markers mapped between Satt114 and Satt510 on chromosome 13 (LG F) revealed that Satt334 was polymorphic between PI 200538 and LD02-4485. The polymorphism between the two soybean genotypes could not be distinguished in 3% agarose gels, so this marker was genotyped with 3% metaphor-agarose gels. Satt334 is located between Satt114 and Satt510 on the genetic map developed by Hill et al. (2009) (FIGS. 1a, b). STSs previously mapped near the Rag2 locus were re-sequenced to saturate the SSR map and to further define the location of Rag2 on chromosome 13. Thirty-four out of the 35 STSs producing a single PCR product in both parents and were sequenced (Table 3). STS #28 was the exception and failed to produce an amplification product. Sixteen of the STSs were found to have 29 SNPs and 33 small insertion/deletions (INDELs). Among these 16 STSs, 8 (#1, #9, #20, #23, #24, #25, #34, #1485) contained SNPs that were appropriate for the development of MCA or TaqMan SNP marker assays (Tables 4, 5). The eight SNP markers developed from re-sequencing the STSs were incorporated onto the genetic map developed by Hill et al. (2009). All SNP markers were mapped between Satt510 and Sat_{—}234. Rag2 was mapped between the SNP markers #1 and #1485 and co-segregated with SNP #20 (FIG. 1b). This resulted in narrowing the genetic interval containing the gene from 10 to 1.6 cM in length using the 95 F2:3 lines from the LD02-4485 9 (Ina 9 PI 200538) population (FIGS. 1a, b).

### Fine Mapping of Rag2 Using SNP Markers Developed by Re-Sequencing

To further define the position of Rag2, SNP marker analyses and SA resistance tests were conducted on progeny plants from the five lines (18, 32, 86, 162, and 181) identified in 2008 as having recombination events between SNP markers #1 and #20 or #20 and #1485. The lines 18, 32, and 86 had recombination events between #20 and #1. The SA resistance segregation among plants in the progeny tests were consistent with the segregation of #20 and #1485, but not #1, suggesting that Rag2 is likely between #20 and #1485 (Table 2).

Direct re-sequencing in the interval between #20 and #1485 was then done to identify additional SNPs that could be used to refine the map position of Rag2. Although one target amplification primer pair was designed for each 10 kb within the interval, some regions did not have sufficient sequence information suitable to design target amplification primers as a result of the draft quality of the soybean genome at the time. A total of 27 primer pairs were designed with thirteen of the 27 primer pairs producing a single PCR product for both parents. Through sequencing of the PCR products from both parents, SNP markers were developed for seven primer pairs. In addition, the two primer pairs KS8 and KS10 produced a single PCR product from only LD02-4485 DNA and failed to amplify a product from PI 200538 and were used as a dominant marker in genotyping the recombinant lines. By testing all progeny plants from the greenhouse resistance evaluations with these dominant markers, lines derived from heterozygous and homozygous susceptible plants could be distinguished (Table 2).

Individual plants from the lines 162 and 181, which were identified as having recombination between SNP markers #20 and #1485, were genotyped with the nine new markers (Table 2). Line 162 was segregating for SNP markers from KS5 to #1 alleles and homozygous resistant from KS7 to #1485. No significant association between the segregation of aphid resistance and the SNP marker KS5 was observed for this line (Table 2) and all tested plants had a resistant phenotype. The results for line 162 show that Rag2 must be to the left of KS5 in the genetic map.

The left, border of the position of Rag2 was refined by analysis of line 181. This line was segregating for SNP markers from KS12 to #1 and homozygous resistant for markers from #1485 to KS14. There was a highly significant association (P\0.0001) between aphid resistance and the segregation of the SNP marker KS12 in the line (Table 2), showing that Rag2 was segregating and therefore to the right of KS14. A BLAST analysis indicated that the SNP detected by the marker KS5 is located at 29,266,469 bp and the SNP detected by the marker KS14 is located at 29,123,397 bp on chromosome 13. Therefore, the genomic region containing the gene was narrowed to an interval approximately 143 kb in length (Table 2; FIG. 1c) based on the Williams 82 sequence.

To further refine the position of Rag2 within the 143-kb region, re-sequencing was done to identify additional DNA markers within the region. Nine target amplification primer pairs were designed for re-sequencing between KS7 and KS12. Of the nine primer pairs, three did not produce a PCR product from PI 200538 and two did not produce a PCR product from both parents. This poor success rate was likely the result of low sequence homology between the Williams 82 sequence, which was used to design the primers, and LD00-4485 and PI 200538, the parent used to test the primers. Four primer pairs produced products from both parents and these products were sequenced. The sequences of three products contained too many SNPs and INDELs which made it impossible to develop SNP assays.

A SNP assay was successfully developed from only the remaining SNP marker (KS9-3). A total of 2,632 BC3F2, F2, and BC4F2 plants grown in the field at Urbana, Ill. were tested with the markers KS7 and KS12 to find additional recombination events within the interval. Three plants were identified with recombinations in the interval and the lines K16, K31, and K37 were derived from these recombinant plants. Progeny plants from these lines were genotyped with all SNP markers and tested for SA resistance. The line K37 was segregating for SNP markers from KS9-3 to #1 and was fixed for markers #1485 to KS10. There was a highly significant (P\0.0001) association between these segregating SNP markers and SA resistance, indicating that Rag2 was to the right of KS10 (Table 2). Lines KS16 and KS31 were segregating for SNP marker alleles from #1485 to KS9-3 and homozygous resistant from KS8 to #1. Because there was no significant association between aphid resistance and segregation for the SNP marker KS9-3 for these lines, Rag2 must be to the right of KS9-3. A BLAST analysis revealed that the SNP marker KS9-3 was located at 29,212,318 bp on soybean chromosome 13 (LG F). These results indicated that Rag2 maps within a 54-kb region defined by KS9-3 and KS5 on soybean chromosome 13 (Table 2; FIG. 1c).

Rag2 Interval in Aphid-Resistant Soybean Accession No. PI 200538

The region mapping between two SNP markers, KS9-3 and KS5 (Kim et al. 2010) in Soybean Accession No. PI 200538 was cloned as follows: Whole-genome shotgun sequencing of a soybean breeding line LD09-15087a, a near-isogenic line (NIL) that harbors Rag2, was conducted using Illumina technology. 1.5 μg of genomic DNA was sequenced using the IIlumina HiSeq 2000 instrument with 100 bp paired-end sequencing. De novo assembly of the genome of the Rag2-carrying NIL was done by ABySS (Simpson, J. T. et al., 2009) and candidate genes for Rag2 were selected using BLASTN analysis.

An LD09-15087a fosmid library was constructed using the CopyControl Fosmid Library Production Kit (Epicentre, USA). 20 μg of the size-fractionated DNA was used for end-repair. 35-45 kb fragment pools of DNA were cloned in the pCC1FOS™ Vector. Ligated DNA was packaged using the MaxPlax™ Lambda Packaging Extracts and transformed into the Phage T1-Resistant EPI 300™-T1R E. coli strain. Two fosmid clones of 42 and 45 kb were identified by PCR-based pool screening from the interval sequence and their end sequences. The fosmid clones were sequenced using a Roche 454/GS FLX+ system (Roche).

The genetic interval for Rag2 was completed using overlapping fosmid clones and Illumina assembly contigs.

Genes and exons were identified within the Rag2 interval as shown in Table 6 and FIG. 2. Genes 2 and 3 were identified as nucleotide-binding site-leucine-rich repeat regions, and having homologies to the Glyma13g26000 interval with rearrangements and thus are claimed herein as conferring Rag2 aphid resistance. The Rag2 interval [SEQ ID NO:65] comprising 48740 base pairs. Base pair 1 corresponds to base pair 29212274. Base pair 48740 corresponds to base pair 29266554 on chromosome 13 in Glycine max v1.0 genome. Genes 1, 2, 3, 7, and 9 do not have introns.

### DISCUSSION

The SA-resistance gene Rag2 from PI 200538 was fine mapped in this study. Mian et al. (2008b) also mapped a single dominant gene named Rag2 from PI 243540 to the same region on soybean chromosome 13 (LG F). Because these genes map to the same region and they both show resistance to SA biotype 2, it is likely that both sources have a resistance allele at the Rag2 locus. Our mapping efforts were greatly accelerated by the availability of the public sequence of the soybean genome (Schmutz et al. 2010). This sequence information was especially valuable in discovering SNPs through direct re-sequencing of target regions, determining the position of markers on the physical map, and identifying candidate genes in the region where the gene is located.

Our work identified the 54-kb region containing the Rag2 locus on the aphid susceptible Williams 82 89 assembly (Glyma1) and predicted the presence of seven candidate genes. Of these genes, Glyma13g26000 was the only nucleotide-binding site (NBS)-leucine-rich repeat (LRR) candidate gene and was therefore selected as containing the Rag2 gene based on the Williams 82 sequence. Glyma13g26000 encodes a F-Box/LRR protein and shares significant homology with Arabidopsis and soybean sequences encoding disease resistance proteins such as the coiled-coil (CC)-NBS-LRR or toll-interleukin receptor-NBS-LRR classes of genes. Wang et al. (2005) demonstrated that the soybean F-box protein gene GmCOI1 mediates Jasmonate (JA) that regulates plant defense and fertility in Arabidopsis. Li et al. (2008) suggested that JA-, ethylene-, and salicylic acid (SA)-regulated signaling pathways were at least partially activated by aphid feeding on soybean.

Our subsequent work further identified the Rag2 interval in aphid-resistance variety PI 200538. The full Rag2 interval sequence [SEQ ID NO 65] contains two NBS-LRR genes, labeled Gene 2 and Gene 3 specifically claimed herein as contributing to aphid resistance.

The majority of cloned resistance genes are members of the NBS-LRR gene family. Cloned NBS-LRR genes that confer aphid resistance include the Mi gene, which controls root-knot nematode (Meloidogyne incognita) and potato aphid (Macrosiphum euphorbiae) resistance in tomato (Lycopersion esculentum Mill.) (Milligan et al. 1998; Rossi et al. 1998), and the Vat gene, which confers resistance to A. gossypii in melon (Cucumis melo L.) (Brotman et al. 2002; Dogimont et al. 2009). In addition, an aphid-resistance gene in Medicago truncatula Gaertner was mapped to a NBS-LRR cluster region (Klingler et al. 2005).

A prominent aspect of the genome organization of the NBS-LRR gene family is that members tend to occur in localized clusters (Martin et al. 2003). Such clustering is seen both for resistance genes specific for different races of the same pathogen (Hulbert and Bennetzen 1991) and for resistance genes conferring resistance to unrelated pathogens (Witsenboer et al. 1995). For instance, three NBSLRR genes were interspersed in a 213-kb region at the MIa locus in barley, which controls resistance to multiple strains of powdery mildew (Wei et al. 1999). In the 84-kb region between KS10 and KS5, where Rag2 is located, three NBS-LRR genes are present. These include Glyma13g26000, first designated Rag2, the Rpg1-b gene, which confers resistance to Pseudomonas syringae pv. glycine (Ashfield et al. 2003), and a third NBS-LRR candidate gene of unknown function. Both Rpg1-b and the third gene are outside the 54-kb region containing Rag2, so they were not deemed to be Rag2. Our research on fine mapping Rag1 also showed that the 115-kb region including Rag1 contains two NBS-LRR genes (Kim et al. 2010).

A major peanut root-knot nematode [M. arenaria (Neal) Chitwood]-resistant QTL was previously mapped to the same region as Rag2 on soybean chromosome 13 using PI 200538 as a resistance source (Tamulonis et al. 1997). The mapping of both aphid and nematode resistance to the same region on chromosome 13 from PI 200538 suggests that Rag2 could be analogous to the Mi gene in conferring resistance to both aphids and nematodes. Further work is needed to determine whether peanut root-knot nematode resistance maps to the same position as Rag2. Tamulonis et al. (1997) also mapped a minor peanut root-knot nematode-resistance QTL to chromosome 15 (LG E) and there is evidence of homology between the Rag2 region and the region on chromosome 15 that contains this minor QTL. This homology is shown by the STS GF097621, which was used to develop the SNP marker #1485. When the SNP was developed through re-sequencing, the STS was located on soybean chromosome 13 in SoyBase (http://soybase.org/) and the Williams 82 89 draft assembly (Glyma1) (verified February 2009), and its assembly sequence was perfectly matched to chromosome 13 and partly (85%) matched chromosome 15 (LG E). SNP marker #1485 also was mapped on soybean chromosome 13 through linkage analysis in our research when we used LOD score 5.0 or higher in JoinMap 3.0. At present, however, the location of the STS has been repositioned to between 4,130,5182-4,130,5716 bp onto chromosome 15 in SoyBase (http://soybase.org/) and Phytozome (http://www.phytozome.net), which is between the SSR markers Satt491 and Satt685 (1,966,3839-4,827,3769 bp). This is the same interval that Tamulonis et al. (1997) mapped the minor peanut root-knot nematode QTL from PI 200538 (GmComposite 2003, http://soybase.org/) indicating regions that include the STS on chromosome 13 and 15 have high homology or are duplicated. This homology is supported by Shoemaker et al. (1996) who reported that an extensive region of homology on soybean chromosomes 13 and 15 exist. Hayes et al. (2000) also reported that two NBS-LRR classes of disease-resistance genes identified by the same probe were mapped on soybean chromosome 13 and 15 and these genes have strong homology. These results suggest that the minor QTL for peanut root-knot nematode resistance on chromosome 15 could be a homolog of the major QTL on chromosome 13.

The high-resolution genetic and physical map of the Rag2 locus facilitates MAS for this gene from PI 200538 in soybean breeding programs as it has resulted in the identification of SNP markers closely linked to it. The introgression of this gene into new cultivars is important as it provides resistance to SA biotypes 1 and 2. The SNP markers we developed are especially useful in MAS and pyramiding of SA-resistance genes because of their very close proximity to the gene and the availability of efficient SNP marker detection assays. In addition, the identification of the physical location of Rag2 on the soybean chromosome greatly facilitates the cloning and functional characterization of the gene. The cloning of Rag2 improves our understanding of SA defense mechanisms in soybean plants. This information also can be applied to compare the function of this gene to other SA-resistance genes such as Rag1 or cloned insect resistance genes in other species.

Our subsequent work further identified the Rag2 interval in aphid-resistance variety PI 200538. The full Rag2 interval sequence [SEQ ID NO 65] contains two NBS-LRR genes, labeled Gene 2 and Gene 3 specifically claimed herein as contributing to aphid resistance.

Methods for analysis, breeding and the like found in U.S. Pat. Nos. 7,994,389 and 7,928,286, and PCT Patent Publication No. WO 2011/097492 can be adapted for use with the markers and trait described herein.

TABLE 3
Information on the 35 STSs on chromosome 13
(LG F) (Hyten et al. 2009) that were sequenced
during the fine mapping of Rag2

| | GenBank | NCBI-dbSNP | Consensus |
|---|---|---|---|
| Marker | accession | name of SNP | Map 4.0 |
| name | # of STS | located on STS | Position (cM) |
| #1 | GF097646 | ss4969612 | 52.261 |
| #2 | GF097659 | ss4969648 | 49.924 |
| #3 | GF097679 | ss4969734 | 50.395 |
| #4 | GF091828 | ss107912850 | 51.666 |
| #5 | GF091836 | ss107914023 | 51.936 |
| #6 | GF091900 | ss107912922 | 55.317 |
| #7 | GF091960 | ss107912982 | 55.858 |
| #8 | GF092024 | ss107913046 | 56.031 |
| #9 | GF092100 | ss107913125 | 50.103 |
| #10 | GF092140 | ss107913165 | 54.149 |
| #11 | GF092174 | ss107913199 | 56.171 |
| #12 | GF092326 | ss107913351 | 52.389 |
| #13 | GF093742 | ss107917482 | 48.349 |
| #14 | GF092414 | ss107913443 | 49.424 |
| #15 | GF092564 | ss107913594 | 56.94 |
| #16 | GF092576 | ss107918675 | 52.338 |
| #17 | GF097352 | ss107912576 | 54.374 |
| #18 | GF093970 | ss107918696 | 49.932 |
| #19 | GF092628 | ss107913658 | 54.078 |
| #20 | GF094005 | ss107918836 | 56.609 |
| #21 | GF092655 | ss107913685 | 51.598 |
| #22 | GF097432 | ss107912657 | 53.202 |
| #23 | GF097440 | ss107912665 | 54.922 |
| #24 | GF094366 | ss107920248 | 49.32 |
| #25 | GF094412 | ss107920435 | 55.989 |
| #26 | GF094471 | ss107920639 | 56.243 |
| #27 | GF097393 | ss107912618 | 53.202 |
| #28 | GF094141 | ss107919283 | 51.602 |
| #29 | GF094142 | ss107919284 | 50.707 |
| #30 | GF094701 | ss107921354 | 54.078 |
| #31 | GF096278 | ss107927651 | 55.781 |
| #32 | GF096883 | ss107929664 | 53.202 |
| #33 | GF096992 | ss107929998 | 52.218 |
| #34 | GF097147 | ss107930597 | 51.896 |
| #1485 | GF097621 | ss4969627 | NA |

TABLE 4
Sequences of target amplification primers and
MCA sensor probes used for SNP genotyping

| Marker | | | |
|---|---|---|---|
| name | Type | Sequences | SEQ. ID NO. |
| #1 | Forward | CTCGAAAGGTGAACATGCACCA | SEQ. ID NO: 5 |
| | Reverse | AGAACATTAAGAGATATGGGAAGGAAGTA | SEQ. ID NO: 6 |
| | | G | |
| | Probe | Fluorescein-SPC- | SEQ. ID NO: 7 |
| | | TTAATACACATATAAATTTTGAGAGCATTT | |
| | | AAGT-Phosphate | |
| #9 | Forward | CAAACCAACCAAATGCTCAGAATACACG | SEQ. ID NO: 8 |
| | Reverse | AATGAATAATTCATATGATTAATAGG | SEQ. ID NO: 9 |
| | Probe | Fluorescein-SPC- | SEQ. ID NO: 10 |
| | | AGTGCATGCATACCTTTAGTTGCTGGATT | |
| | | TGAGAT-Phosphate | |
| #20 | Forward | ACATTGCAATCAAAATCAAGATGTAGCTG | SEQ. ID NO: 11 |
| | | G | |
| | Reverse | GACGATTTTGGTTTCTGTGATCTTACGTG | SEQ. ID NO: 12 |
| | Probe | Fluorescein-SPC- | SEQ. ID NO: 13 |
| | | TTCTGTAGCTTCTACCCAAGGGCTAGCCT | |
| | | TATCCA-Phosphate | |
| #23 | Forward | ATGCCAATCCATTCTAAAGT | SEQ. ID NO: 14 |
| | Reverse | GGATCATTGATGGCACGA | SEQ. ID NO: 15 |
| | Probe | Fluorescein-SPC- | SEQ. ID NO: 16 |
| | | TTCTGCAAACATAAACGGATCAAAATATC | |
| | | A-Phosphate | |
| #24 | Forward | CCCCATGGAAATTAAGATTCCTGC | SEQ. ID NO: 17 |
| | Reverse | GCATGAGCACAAAGTTTTTCTTGGC | SEQ. ID NO: 18 |
| | Probe | Fluorescein-SPC- | SEQ. ID NO: 19 |
| | | ATGCCCATGGTTAATTAAGTAAACACATT | |
| | | T-Phosphate | |
| #25 | Forward | GTGTGCATGTGTTTGAACTTTGAAGAGAT | SEQ. ID NO: 20 |
| | | T | |
| | Reverse | ATCACAGAGACATGGAGGTTGCTAT | SEQ. ID NO: 21 |
| | Probe | Fluorescein-SPC- | SEQ. ID NO: 22 |
| | | CTTGTCCTCCTGACTCTCTCCAGGTACTT- | |
| | | Phosphate | |
| #34 | Forward | AGAATATTATGAAGATCAAACATGAACAA | SEQ. ID NO: 23 |
| | Reverse | AATAATGTTTTGTTTAATACTACTTGG | SEQ. ID NO: 24 |
| | Probe | Fluorescein-SPC- | SEQ. ID NO: 25 |
| | | TTTCTCCTTTAAAAATAAGTAGAACCATTT | |
| | | TTTT-Phosphate | |
| KS2 | Forward | CTGCATCAGCTACTTCATGAGGAG | SEQ. ID NO: 26 |
| | Reverse | GGTCTGATTTGCTATTAAACCATCTTCCTT | SEQ. ID NO: 27 |
| | Probe | Fluorescein-SPC- | SEQ. ID NO: 28 |
| | | ACCAGTCCTCTGAAAAAGTGAAGAGAAAT | |
| | | CAACAA-Phosphate | |
| KS4 | Forward | ACCACAAAACAAGCAAATGAGTCACT | SEQ. ID NO: 29 |
| | Reverse | GTGCATGTTCGTTGTGATTTTCCCT | SEQ. ID NO: 30 |
| | Probe | Fluorescein-SPC- | SEQ. ID NO: 31 |
| | | CAATGCACAAGTAGGAAAAATCATCCAAA | |
| | | CGGGAA-Phosphate | |
| KS5 | Forward | CATGGAAGGCTGATAATACAGACATGTAC | SEQ. ID NO: 32 |
| | | C | |
| | Reverse | CGTCGAGCTTAATGCGTGAAGGAAA | SEQ. ID NO: 33 |
| | Probe | Fluorescein-SPC- | SEQ. ID NO: 34 |
| | | GGAAGAGGATGAGGACGCCATCATCGAC | |
| | | ATTCA-Phosphate | |
| KS7 | Forward | CAGGGCAAAGTGTGGAGACAAT | SEQ. ID NO: 35 |
| | Reverse | CAATCCATTATACGCTATACACTCCCCTT | SEQ. ID NO: 36 |
| | | C | |
| | Probe | Fluorescein-SPC- | SEQ. ID NO: 37 |
| | | AGCTAGTTCGATTTTATCAACAATTAGGG | |
| | | TGATGA-Phosphate | |
| KS12 | Forward | ATCAAGCTCACTCCTTATTGAATAAACCT | SEQ ID NO: 38 |
| | Reverse | ACATTGATCCATTATGTTTGCTTAACAAGT | SEQ ID NO: 39 |
| | Probe | Fluorescein-SPC- | SEQ. ID NO: 40 |
| | | AAAGGAATGCATTAGAAACTTATTGCCAC | |
| | | TCCTCA-Phosphate | |
| KS14 | Forward | CTTCCGTCATCCATTAAGAGCAATTCATTT | SEQ. ID NO: 41 |
| | Reverse | TGGATGCAGAGGTTGTGTATGTGGTTTAG | SEQ. ID NO: 42 |
| | Probe | Fluorescein-SPC- | SEQ. ID NO: 43 |
| | | AGTTGTTAACATGACAAGAGGTGAAAGAG | |
| | | ACGAGT-Phosphate | |
| KS16 | Forward | CAGGTTCTTCACTCAAGTTGTTGCT | SEQ. ID NO: 44 |
| | Reverse | AATCAGAATCAGATTGAAAACAAGACACC | SEQ. ID NO: 45 |
| | | A | |
| | Probe | Fluorescein-SPC- | SEQ. ID NO: 46 |
| | | GTCACATTTTGTTTTTGTTGTAATTTTGTT | |
| | | GGA-Phosphate | |

TABLE 5
Sequences of target amplification primers and
TaqMan probes for SNP genotyping

| Marker | | | |
|---|---|---|---|
| Name | Type | Sequence | SEQ ID NO. |
| #1 | Forward | AGAACATTAAGAGATATGGGAAGGAA | SEQ. ID NO: 47 |
| | | GTAGT | |
| | Reverse | GGAACATTACTAAAAACGATATGTCAA | SEQ. ID NO: 48 |
| | | AGTTAGAA | |
| | Probe 1 | ATTTTGAGAGCATTTAAG-VIC | SEQ. ID NO: 49 |
| | Probe 2 | AATTTTGAGAGCTTTTAAG-FAM | SEQ. ID NO: 50 |
| #20 | Forward | AAATCAAGATGTAGCTGGATGGATAA | SEQ. ID NO: 51 |
| | | GG | |
| | Reverse | GCTTTTGCACTTGAATTATTTGTTTTC | SEQ. ID NO: 52 |
| | | TGT | |
| | Probe 1 | CCCTTGGGTAGAAGC-VIC | SEQ. ID NO: 53 |
| | Probe 2 | CCCTTGGATAGAAGC-FAM | SEQ. ID NO: 54 |
| #1485 | Forward | GCATAGAAATTTACACATCCATCAACC | SEQ. ID NO: 55 |
| | | AT | |
| | Reverse | CGTTTGGGAATAGCTTACAAGCTT | SEQ. ID NO: 56 |
| | Probe 1 | ACTCTACCCTGACAATAG-VIC | SEQ. ID NO: 57 |
| | Probe 2 | CTCTACCCTCACAATAG-FAM | SEQ. ID NO: 58 |
| KS9-3 | Forward | ACGTCAAGTGATGACTTAACACTTGT | SEQ. ID NO: 59 |
| | Reverse | AGAAGTAGGAAGGACAAAACTTGAAT | SEQ. ID NO: 60 |
| | | ATAAAGAAAAA | |
| | Probe 1 | ATCATTAGAAAACGAAATAA-VIC | SEQ. ID NO: 61 |
| | Probe 2 | ATCATTAGAAAACAAAATAA-FAM | SEQ. ID NO: 62 |

TABLE 6
Rag2 Interval Genes and Exons

| | | Sequence | | Predicted gene |
|---|---|---|---|---|
| Type | Name | intervals | Direction | annotations |
| Gene | 1 | 1294-1696 | Forward | |
| Gene | 2 | 7377-7976 | Forward | NBS-LRR |
| Gene | 3 | 9421-11097 | Forward | NBS-LRR |
| Gene | 4 | 16204-17803 | Reverse | Mitochondrial Inner |
| | | | | Membrane Protein |
| Exon | EXON1 of Gene4 | 16726-16803 | Reverse | |
| Exon | EXON2 of Gene4 | 16204-16617 | Reverse | |
| Gene | 5 | 19326-22276 | Reverse | Mitochondrial Inner |
| | | | | Membrane Protein |
| Exon | EXON1 of Gene5 | 22220-22276 | Reverse | |
| Exon | EXON2 of Gene5 | 22220-22276 | Reverse | |
| Exon | EXON3 of Gene5 | 20251-20523 | Reverse | |
| Exon | EXON4 of Gene5 | 19891-20001 | Reverse | |
| Exon | EXONS of Gene5 | 19326-19795 | Reverse | |
| Gene | 6 | 42008-42295 | Forward | |
| Exon | Exon1 of Gene6 | 42008-42039 | Forward | |
| Exon | Exon2 | 42225-42295 | Forward | |
| Gene | 7 | 43619-44180 | Forward | |
| Gene | 8 | 46451-47240 | Reverse | |
| Exon | Exon1 of Gene8 | 46697-47240 | Reverse | |
| Exon | Exon2 of Gene8 | 46451-46599 | Reverse | |
| Gene | 9 | 48415-48738 | Forward | |

When a compound is claimed as a composition of matter, it should be understood that compounds known in the art including the compounds or sequences disclosed in the references disclosed herein are not intended to be claimed by themselves. When a Markush group or other grouping is used herein, all individual members of the group and all combinations and subcombinations possible of the group are intended to be individually included in the disclosure.

One of ordinary skill in the art will appreciate that methods, genetic or other elements, starting materials, molecular biological and agronomic methods, other than those specifically exemplified herein can be employed in the practice of the methods hereof without resort to undue experimentation. All art-known functional equivalents, of any such methods, genetic or other elements, starting materials, molecular and agronomic methods, are intended to be included within the scope of the claims. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive and open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising,” particularly in a description of components of a composition or in a description of elements of a device, is understood to encompass those compositions and methods consisting essentially of and consisting of the recited components or elements. The methods and constructs illustratively described herein suitably can be practiced in the absence of any element or elements, limitation or limitations which are not specifically disclosed herein.

In general the terms and phrases used herein have their art-recognized meaning, which can be found by reference to standard texts, journal references and contexts known to those skilled in the art.

Although methods and constructs have been described in detail for purposes of clarity and understanding, it will be clear to those skilled in the art that equivalent cultivars, markers, and methods can be practiced within the scope of the claims hereof. And although the description herein contains certain specific information and examples, these should not be construed as limiting the scope of the claims, but as merely providing illustrations of some of the embodiments hereof. Thus, additional embodiments are within the scope of the claims.

### REFERENCES

### Patent Publications

U.S. Pat. No. 7,994,389, Hill et al., issued Aug. 9, 2011 for Soybean Genes for Resistance to Aphis Glycines
U.S. Pat. No. 7,928,286, Hill et al., issued Apr. 19, 2011 for Soybean Gene for Resistance to Aphis glycines
PCT Patent Publication No. WO 2011/097492 Hudson, M., et al., published Aug. 11, 2011 for A DNA Sequence that Confers Aphid Resistance in Soybean

### Non-Patent Literature

Ashfield T, Bocian A, Held D, Henk A D, Marek L F, Danesh D. Penuela S, Meksem K, Lightfoot D A, Young N D, Shoemaker R C, Innes R W (2003) Genetic and physical localization of the soybean Rpg1-b disease resistance gene reveals a complex locus containing several tightly linked families of NBS-LRR genes. Mol Plant Microbe Interact 16:817-826
Bell-Johnson B, Garvey G, Johnson J, Lightfoot D, Meksem K (1998) Biotechnology approaches to improving resistance to SCN and SDS: methods for high-throughput marker-assisted selection. Soybean Genet Newsl 25:115-117
Brotman Y, Silberstein L, Kovalski I, Perin C, Dogimont C, Pitrat M, Klingler J, Thompson G A, Perl-Treves R (2002) Resistance gene homologous in melon are linked to genetic loci conferring disease and pest resistance. Theor Appl Genet. 104:1055-1063
Choi I Y, Hyten D L, Matukumalli L K, Song Q, Chaky J M, Quigley C V, Chase K. Lark K G, Reiter R S, Yoon M S, Hwang E Y, Yi S I, Young N D, Shoemaker R C, van Tassell C P, Specht J E, Cregan P B (2007) A soybean transcript map: gene distribution, haplotype and single-nucleotide polymorphism analysis. Genetics 176:685-696
Cregan P B, Quigley C V (1997) Simple sequence repeat DNA marker analysis. In: Caetano-Anolles G, Gresshoff P M (ed) DNA markers: Protocols, applications and overview. John. Wiley & Sons, New York, pp 173-185
Dogimont C. Bendahmane A., Pitrat, M, Burget-Bigeard E, Hagen L, Le Menn A, Pauquet J, Rousselle P, Caboche M, Chovelon V (2009) Gene resistant to Aphis gossypii. U.S. Pat. No. 7,576,264. Date issued: 18 Aug. 2009
Hayes A J, Yue Y G, Saghai Maroof M A (2000) Expression of two soybean resistance gene candidates shows divergence of paralogous single-copy genes. Theor Appl Genet. 101:789-795
Hill, C. B., Crull, L., Herman, T., Voegtlin, D. J., and Hartman, G. L. 2010. A new soybean aphid (Hemiptera: Aphididae) biotype identified. J Econ Entomol. 2010 April; 103(2):509-15.
Hill C B, Kim K S, Crull L, Diers B W, Hartman G L (2009) Inheritance of resistance to the soybean aphid in soybean PI 200538. Crop Sci 49:1193-1200
Hill C B, Li Y, Hartman G L (2004a) Resistance to the soybean aphid in soybean germplasm. Crop Sci 44:98-106
Hill C B, Li Y, Hartman G L (2004b) Resistance to Glycine species and various cultivated legumes to the soybean aphid (Homoptera: Aphididae). J Econ Entomol 97:1071-1077
Hill C B, Li Y, Hartman G L (2006a) A single dominant gene for resistance to the soybean aphid in the soybean cultivar Dowling. Crop Sci 46:1601-1605
Hill C B, Li Y, Hartman G L (2006b) Soybean aphid resistance in soybean Jackson is controlled by a single dominant gene. Crop Sci 46:1606-1608
Honeycutt R J, Sobral B W S, Keim P, Irvine J E (1992) A rapid DNA extraction method for sugarcane and its relatives. Plant Mol Biol Rep 10:66-72
Hulbert S H, Bennetzen J L (1991) Recombination at the RP1 locus of maize. Mol Gen Genet. 226:377-382
Hyten D L, Choi I-Y, Song Q, Specht J E, Carter T E, Shoemaker R C, Hwang E-Y, Matukumalli L K, Cregan P B (2009) A high density integrated genetic linkage map of soybean and the development of a 1,536 Universal Soy Linkage Panel for QTL mapping, Crop Science. 50:960-968
Kaczorowski K A, Kim K S, Diers B W, Hudson M E (2008) Microarray-based genetic mapping using soybean near-isogenic lines and generation of SNP markers in the Rag1 aphid-resistance interval. Plant Genome 1:89-98
Kang S T, Mian M A R, Hammond R B (2008) Soybean aphid resistance in PI 243540 is controlled by a single dominant gene. Crop Sci 48:1744-1788
Kim K S, Hill C B, Hartman G L, Mian M A R, Diers B W (2008) Discovery of soybean aphid biotypes. Crop Sci 48:923-928
Kim K S, Bellendir S. Hudson K A, Hill C B, Hartman G L, Hyten D, Hudson M E, Diers B W (2010) Fine mapping the soybean aphid resistance gene Rag1 in soybean. Theor Appl Genet. 120(5):1063-71
Kim, K S, Hill C B, Hartman G L, Hyten D, Hudson M E, Diers B W (2010) Fine mapping of the soybean aphid-resistance gene Rag2 in soybean PI 200538 Theor Appl Genet. 121:599-610
Klingler J, Creasy R, Gao L L, Nair R M, Calix A S, Jacob H S, Edwards O R, Singh K B (2005) Aphid resistance in Medicago truncatula involves antixenosis and phloem-specific, inducible antibiosis, and maps to a single locus flanked by NBS-LRR resistance gene analogs. Plant Physiol 137:1445-1455
Kobe B, Kajava A V (2001) The leucine-rich repeat as a protein recognition motif. Curr Opin Struct Biol 11:725-732
Kobe B, Deisenhofer J (1994) The leucine-rich repeat: a versatile binding motif. Trends Biochem Sci 19:415-421
Li Y, Hill C B, Carlson S R, Diers B W, Hartman G L (2007) Soybean aphid resistance in the soybean cultivars Dowling and Jackson map to linkage group M. Mol Breed 19:25-34
Li Y. Hill C B, Hartman G L (2004) Effect of three resistant soybean genotypes on the fecundity, mortality, and maturation of soybean aphid (Homoptera: Aphididae). J Econ Entomol 97:1106-1111
Li Y, Zou J, Li M, Bilgin D D, Vodkin L O, Hartman G L, Clough S J (2008) Soybean defense responses to the soybean aphid. New Phytologist 179(1):185-195
Martin G B, Bogdenove A J, Sessa G (2003) Understanding the functions of plant disease resistance proteins. Annu Rev of Plant Biol 54:23-61
Mensah C, DiFonzo C, Wang D (2008) Inheritance of soybean aphid resistance in PI 567541B and PI 567598B. Crop Sci 48:1759-1763
Mian M A R, Hammond R B, St Martin S K (2008a) New plant introductions with resistance to the soybean aphid. Crop Sci 48:1055-1061
Mian M A R, Kang S T, Beil S E, Hammond R B (2008b) Genetic linkage mapping of the soybean aphid resistance gene in PI 243540. Theor Appl Genet. 117:955-962
Michel A P, Zhang W., Jung J K, Kang, S T, Mian M A R (2009) Population genetic structure of Aphis Glycines, Environ Entomol 38:1301-1311
Milligan S B, Bodeau J, Yaghoobi J, Kaloshian I, Zabel P, Williamson V M (1998). The root knot nematode resistance gene Mi from tomato is a member of the leucine zipper, nucleotide binding, leucine-rich-repeat family of plant genes. Plant Cell 10:1307-1320
Nickell C D, Noel G R, Cary T R, Thomas D J, Leitz R A (1999) Registration of ‘Ina’ Soybean. Crop Sci 39:1533
Preez F B (2005) Tracking nucleotide-binding-site-leucine-rich-repeat resistance gene analogues in the wheat genome complex. Dissertation. University of Pretoria
Rossi M, Goggin F L, Milligan S B, Kaloshian I, Ullman D E, Williamson V M (1998) The nematode resistance gene Mi of tomato confers resistance against the potato aphid. Proc Natl Acad Sci USA 95: 9750-9754
SAS Institute (2002) The SAS system for Windows. Release 9.00. SAS Institute, Cary Schmutz J, Cannon S, Schlueter J, Ma J, Mitros T, Nelson W, Hyten D, Song Q, Thelen J, Cheng J, Xu D, Helisten U, May G, Yu Y, Sakurai T, Umezawa T, Bhattacharyya M, Sandhu D, Valliyodan B, Lindquist E, Peto M, Grant D, Shu S, Goodstein D, Barry K, Futrell-Griggs M, Du J, Tian Z, Zhu L, Gill N, Joshi T, Libault M, Sethuraman A, Zhang X-C, Shinozaki K, Nguyen H, Wing R, Cregan P, Specht J, Grimwood J, Rokhsar D, Stacey G, Shoemaker R, Jackson S (2010) Genome seqeunce of the paleopolyploid soybean. Nature. (463): 178-183.
Shoemaker R C, Polzin K, Labate J, Specht J, Brummer E C, Olson T, Young N, Concibido V, Wilcox J. Tamulonis J P (1996) Genome duplication in soybean (Glycine subgenus soja). Genetics 144:329-338
Simpson, J T, K. Wong, S. D. Jackman, J. E. Schein, S. J. M. Jones, I. Birol (2009), Genome Res 19, 1117-1123
Song Q J, Marek L F, Shoemaker R C, Lark K G, Concibido V C, Delannay X, Specht J E, Cregan P B (2004) A new integrated genetic linkage map of the soybean. Theor Appl Genet. 109:122-128
Tamulonis J P, Luzzi B M, Hussey R S, Parrott W A, Boemia H R (1997) DNA marker analysis of loci conferring resistance to peanut root-knot nematode in soybean. Theor Appl Genet. 95:664-670
Traut T W (1994) The functions and consensus motifs of nine types of peptide segments that form different types of nucleotide-binding sites. Eur J Biochem 222:9-19
Van Ooijen J W, Voorrips RE (2001) JoinMap 3.0 software for the calculation of genetic linkage maps. Plant Research Internation, Wageningen
Voegtlin D J (2008) United States Soybean Aphid Commentary [Online]. Available at http://sba.ipmpipe.org/cgi-bin/sbr/public.cgi?host=All%20Legumes/Kudzu&pest=soybean_aphid (verified 18 Jun. 2009)
Wang Z, Dai L, Jiang Z, Peng W, Zhang L, Wang G, Xie D (2005) GmCOI1, a soybean F-box protein gene, shows ability to mediate jasmonate-regulated plant defense and fertility in Arabidopsis. Mol Plant Microbe Interact 18:1285-1295
Wei F, Gobelman-Wemer K, Morroll S M, Kurth J, Mao L, Wing R, Leister D, Schulze-Lefert P, W is RP (1999) The MIa (powdery mildew) resistance cluster is associated with three NBS-LRR gene families and suppressed recombination within a 240-kb DNA interval on chromosome 55 (1 HS) of barley. Genetics 153:1929-1948
Witsenboer H, Kesseli R V, Fortin M G, Stanghellini M, Michelmore R W (1995) Sources and genetic structure of a cluster of genes for resistance to three pathogens in lettuce. Theor Appl Genet. 91:178-188
Zhang G, Gu C, Wang D (2009) Molecular mapping of soybean aphid resistance genes in PI 567541B. Theor Appl Genet. 118:473-482

1. An isolated synthetic or recombinant DNA molecule selected from the group consisting of:
DNA molecules having a sequence selected from the group consisting of: the Rag2 Interval nucleic acid sequence [SEQ ID NO:65]; and base pairs 1294-1696 (Gene 1); 7377-7976 (Gene 2); 9421-11097 (Gene 3); 16204-17803 (Gene 4); 19326-22276 (Gene 5); 42008-42295 (Gene 6); 43619-44180 (Gene 7); 46451-47240 (Gene 8); and 48415-48738 (Gene 9) of said SEQ ID NO:65; and RNA molecules having sequences of RNA molecule expressed by said DNA molecules; which DNA and RNA molecules encode a polypeptide that is capable of conferring aphid resistance to a soybean plant or participating in conferring or enhancing aphid resistance to a soybean plant when expressed by the plant;
complements of said DNA and RNA molecules; and
polypeptide molecules encoded by said DNA and RNA molecules.

2. A polypeptide molecule of claim 1.

3. The DNA or RNA molecule of claim 1 encoding a Rag2 gene from soybean variety PI 200538 or PI 243540.

4. The molecule of claim 1 having the DNA sequence of: Gene 2, an RNA expressed by Gene 2, or a polypeptide expressed by Gene 2.

5. The molecule of claim 1 having the DNA sequence of: Gene 3, an RNA expressed by Gene 3, or a polypeptide expressed by Gene 3.

6. A DNA molecule of claim 1 operably linked upstream at the 5′ end to a promoter capable of causing expression of said molecule in a heterologous host plant.

7. A recombinant host cell comprising a nucleic acid molecule of claim 1 wherein said nucleic acid molecule is non-native to said host cell.

8. The recombinant host cell of claim 7 which is a transgenic plant cell.

9. A transgenic plant comprising the transgenic plant cell of claim 8.

10. A method of producing a recombinant polypeptide capable of conferring or participating in conferring aphid resistance on a soybean plant comprising the steps of introducing a nucleic acid molecule of claim 1 into a host cell under conditions that allow expression of the polypeptide.

11. A method for producing an aphid-resistant soybean crop in a field comprising planting the field with crop seeds or plants that are aphid-resistant as a result of said seeds or plants being derived from transformed cells comprising a nucleic acid or polypeptide sequence of claim 1.

12. A method for identifying, in germplasm of a plant, a DNA sequence capable of conferring or contributing to conferring, or enhancing, aphid resistance on said plant, said method comprising amplifying DNA from said germplasm with a nucleic acid probe comprising at least about 10 or at least about 75 consecutive bases of a nucleic acid molecule having a sequence selected from the group consisting of SEQ ID NOS:7, 10, 13, 16, 19, 22, 25, 28, 31, 34, 37, 40, 43, 46, 49, 50, 53, 54, 57, 58, 61, 62, 65, and Genes 1-9, to obtain an amplified DNA molecule capable of hybridizing to a DNA molecule of claim 1,
wherein when the sequences of said amplified DNA molecule are identical to sequences of a DNA molecule of claim 1, said amplified DNA molecule is capable of conferring or contributing to conferring, or enhancing, aphid resistance in said plant.

13. The method of claim 12 wherein said probe consists of the first and/or last 10 to 75 consecutive base pairs of Gene 2.

14. The method of claim 12 wherein said probe consists of the first and/or last 10 to 75 base pairs of Gene 3.

15. The method of claim 12 wherein the germplasm is a soybean germplasm.

16. The method of claim 12 wherein the germplasm is that of a plant species or genus having members which are aphid-susceptible.

17. A nucleic acid probe for use in identifying a nucleic acid encoding a polypeptide conferring or capable of participating in conferring or enhancing aphid resistance on a plant wherein the probe comprises at least about 10 or at least about 75 consecutive bases of a nucleic acid molecule having a sequence selected from the group consisting of SEQ ID NOS:7, 10, 13, 16, 19, 22, 25, 28, 31, 34, 37, 40, 43, 46, 49, 50, 53, 54, 57, 58, 61, 62, 64, 65, and Genes 1-9.

18. A computer storage medium having recorded thereon one or more sequences selected from the group consisting of SEQ ID NOS:1-65 and Genes 1-9, together with information identifying said sequences.

19. A method for determining the presence or absence of a gene for aphid resistance in soybean germplasm comprising analyzing said germplasm by marker-assisted selection (MAS) to:
a. detect a Rag2 aphid-resistance locus that maps to soybean chromosome 13 of said soybean germplasm, wherein said Rag2 locus shows allelic polymorphism between aphid-resistant and aphid-susceptible soybean and is flanked on opposite sides by markers KS5 and KS9-3, wherein the Rag2 locus comprises allelic DNA sequences that control aphid resistance; and
b. determine the presence or absence of an allelic form of DNA linked to the Rag2 gene coding for resistance to Aphis glycines in said germplasm;
said method comprising:
(1) making a first PCR-amplified polymorphic marker fragment from said soybean germplasm; and
(2) making a second PCR-amplified polymorphic marker fragment from soybean germplasm of a plant having aphid resistance conferred by said Rag2 gene,
(i) wherein the second fragment is made by PCR amplification of the same marker that was used to make said first fragment, and
(ii) wherein said second fragment has a size substantially the same as that of a PCR-amplified polymorphic marker fragment of germplasm of aphid-resistant soybean variety PI200538 made using the same marker used to make said first and second fragments;
wherein said gene coding for Rag2 resistance is present in said soybean germplasm when said first fragment is substantially the same size as said second fragment, and wherein said gene is not present in said germplasm when said first fragment is not substantially the same size as said second fragment.

20. A kit for selecting at least one soybean plant by marker-assisted selection of a quantitative trait locus associated with aphid resistance comprising:
(a) primers and/or probes for detecting at least one Aphis glycines resistance-associated marker locus selected from the group consisting of primers or probes comprising at last about 10 or at least about 75 base pairs of at least two markers or genes selected from the group consisting of Markers #1, #9, #20, #23, #24, #25, #34, KS2, KS4, KS5, KS7, KS12, KS14, KS16, #1485, and KS9-3 and Genes 1-9; and markers mapping within about 5 to about 10 cM thereof; and
(b) instructions for using the primers and/or probes for detecting genes and/or marker loci and correlating the genes and/or loci with predicted aphid resistance.


SEQUENCE LISTING (65 sequences)

SEQ ID NO: 1 - DNA, 24 bp, Glycine max
taccctcaaatggacttggtgcct

SEQ ID NO: 2 - DNA, 23 bp, Glycine max
ggcgatggtgatcttgactgtct

SEQ ID NO: 3 - DNA, 24 bp, Glycine max
tcccattacgccgttcagcaagat

SEQ ID NO: 4 - DNA, 24 bp, Glycine max
ggtgtacaaggaaagcccaagact

SEQ ID NO: 5 - DNA, 22 bp, Glycine max
ctcgaaaggtgaacatgcacca

SEQ ID NO: 6 - DNA, 30 bp, Glycine max
agaacattaagagatatgggaaggaagtag

SEQ ID NO: 7 - DNA, 34 bp, Glycine max
ttaatacacatataaattttgagagcatttaagt

SEQ ID NO: 8 - DNA, 28 bp, Glycine max
caaaccaaccaaatgctcagaatacacg

SEQ ID NO: 9 - DNA, 26 bp, Glycine max
aatgaataattcatatgattaatagg

SEQ ID NO: 10 - DNA, 35 bp, Glycine max
agtgcatgcatacctttagttgctggatttgagat

SEQ ID NO: 11 - DNA, 30 bp, Glycine max
acattgcaatcaaaatcaagatgtagctgg

SEQ ID NO: 12 - DNA, 29 bp, Glycine max
gacgattttggtttctgtgatcttacgtg

SEQ ID NO: 13 - DNA, 35 bp, Glycine max
ttctgtagcttctacccaagggctagccttatcca

SEQ ID NO: 14 - DNA, 20 bp, Glycine max
atgccaatccattctaaagt

SEQ ID NO: 15 - DNA, 18 bp, Glycine max
ggatcattgatggcacga

SEQ ID NO: 16 - DNA, 30 bp, Glycine max
ttctgcaaacataaacggatcaaaatatca

SEQ ID NO: 17 - DNA, 24 bp, Glycine max
ccccatggaaattaagattcctgc

SEQ ID NO: 18 - DNA, 25 bp, Glycine max
gcatgagcacaaagtttttcttggc

SEQ ID NO: 19 - DNA, 30 bp, Glycine max
atgcccatggttaattaagtaaacacattt

SEQ ID NO: 20 - DNA, 30 bp, Glycine max
gtgtgcatgtgtttgaactttgaagagatt

SEQ ID NO: 21 - DNA, 25 bp, Glycine max
atcacagagacatggaggttgctat

SEQ ID NO: 22 - DNA, 29 bp, Glycine max
cttgtcctcctgactctctccaggtactt

SEQ ID NO: 23 - DNA, 29 bp, Glycine max
agaatattatgaagatcaaacatgaacaa

SEQ ID NO: 24 - DNA, 30 bp, Glycine max
aataatgttttgtttaatacttttacttgg

SEQ ID NO: 25 - DNA, 34 bp, Glycine max
tttctcctttaaaaataagtagaaccattttttt

SEQ ID NO: 26 - DNA, 24 bp, Glycine max
ctgcatcagctacttcatgaggag

SEQ ID NO: 27 - DNA, 30 bp, Glycine max
ggtctgatttgctattaaaccatcttcctt

SEQ ID NO: 28 - DNA, 35 bp, Glycine max
accagtcctctgaaaaagtgaagagaaatcaacaa

SEQ ID NO: 29 - DNA, 26 bp, Glycine max
accacaaaacaagcaaatgagtcact

SEQ ID NO: 30 - DNA, 25 bp, Glycine max
gtgcatgttcgttgtgattttccct

SEQ ID NO: 31 - DNA, 35 bp, Glycine max
caatgcacaagtaggaaaaatcatccaaacgggaa

SEQ ID NO: 32 - DNA, 30 bp, Glycine max
catggaaggctgataatacagacatgtacc

SEQ ID NO: 33 - DNA, 25 bp, Glycine max
cgtcgagcttaatgcgtgaaggaaa

SEQ ID NO: 34 - DNA, 33 bp, Glycine max
ggaagaggatgaggacgccatcatcgacattca

SEQ ID NO: 35 - DNA, 22 bp, Glycine max
cagggcaaagtgtggagacaat

SEQ ID NO: 36 - DNA, 30 bp, Glycine max
caatccattatacgctatacactccccttc

SEQ ID NO: 37 - DNA, 35 bp, Glycine max
agctagttcgattttatcaacaattagggtgatga

SEQ ID NO: 38 - DNA, 29 bp, Glycine max
atcaagctcactccttattgaataaacct

SEQ ID NO: 39 - DNA, 30 bp, Glycine max
acattgatccattatgtttgcttaacaagt

SEQ ID NO: 40 - DNA, 35 bp, Glycine max
aaaggaatgcattagaaacttattgccactcctca

SEQ ID NO: 41 - DNA, 30 bp, Glycine max
cttccgtcatccattaagagcaattcattt

SEQ ID NO: 42 - DNA, 29 bp, Glycine max
tggatgcagaggttgtgtatgtggtttag

SEQ ID NO: 43 - DNA, 35 bp, Glycine max
agttgttaacatgacaagaggtgaaagagacgagt

SEQ ID NO: 44 - DNA, 25 bp, Glycine max
caggttcttcactcaagttgttgct

SEQ ID NO: 45 - DNA, 30 bp, Glycine max
aatcagaatcagattgaaaacaagacacca

SEQ ID NO: 46 - DNA, 33 bp, Glycine max
gtcacattttgtttttgttgtaattttgttgga

SEQ ID NO: 47 - DNA, 31 bp, Glycine max
agaacattaagagatatgggaaggaagtagt

SEQ ID NO: 48 - DNA, 35 bp, Glycine max
ggaacattactaaaaacgatatgtcaaagttagaa

SEQ ID NO: 49 - DNA, 18 bp, Glycine max
attttgagagcatttaag

SEQ ID NO: 50 - DNA, 19 bp, Glycine max
aattttgagagcttttaag

SEQ ID NO: 51 - DNA, 28 bp, Glycine max
aaatcaagatgtagctggatggataagg

SEQ ID NO: 52 - DNA, 30 bp, Glycine max
gcttttgcacttgaattatttgttttctgt

SEQ ID NO: 53 - DNA, 15 bp, Glycine max
cccttgggtagaagc

SEQ ID NO: 54 - DNA, 15 bp, Glycine max
cccttggatagaagc

SEQ ID NO: 55 - DNA, 29 bp, Glycine max
gcatagaaatttacacatccatcaaccat

SEQ ID NO: 56 - DNA, 24 bp, Glycine max
cgtttgggaatagcttacaagctt

SEQ ID NO: 57 - DNA, 18 bp, Glycine max
actctaccctgacaatag

SEQ ID NO: 58 - DNA, 17 bp, Glycine max
ctctaccctcacaatag

SEQ ID NO: 59 - DNA, 26 bp, Glycine max
acgtcaagtgatgacttaacacttgt

SEQ ID NO: 60 - DNA, 37 bp, Glycine max
agaagtaggaaggacaaaacttgaatataaagaaaaa

SEQ ID NO: 61 - DNA, 20 bp, Glycine max
atcattagaaaacgaaataa

SEQ ID NO: 62 - DNA, 20 bp, Glycine max
atcattagaaaacaaaataa

SEQ ID NO: 63 - protein, 1294 aa, Glycine max
MALELVGGALLSAFLQAAFQKLASHQIRDFFRGRKLDQKLLNNLEIKLNSIQALADDAELKQFRDPRVRNWLLKVKDAVFDAEDLLDEIQHEISKCQVDAEAEAESQTCTCKVPNFFKSSPVSSFYKEIKSRMEQVLEDLENLASQSGYLGLKNASGVGSGFGGAVSQQSQSTSLLVERVIYGRDDDKEMIFNWLTSDIDNCNKPSIFSIVGMGGLGKTTLAQHVFNDPRIENKFDIKAWVCVSDEFDVFNVTRTILEAVTKSTDDSRNREMVQGRLKEKLTGKRFFLVLDDVWNRNQKEWEALQTPLNDGAPGSKIVVTTRDKKVASIVGSNKTHCLELLQDDHCWQLLAKHAFQDDSHQPNADFKEIGTKIVAKCKGLPLALTTIGSLLHQKSSISEWEGILKSEIWEFSEEDSSIVPALALSYHHLPSRLKRCFAYCALFPKDYRFGKEGLIQLWMAENFLQCHQQSRSPEEVGEQYFNDLLSRSFFQQSSNIEGKPFVMHDLLNDLAKYVCGDFCFRLEDDQPKHIPKTTRHFSVASNHVKCFDGFGTLYNAERLRTFMSLSEETSFHNYSRWYCKMSTRELFSKFKFLRVLSVSDYSNLTELPDSVGNLKYLHSLDLSNTGIEKLPESTCSLYNLQILKLNGCKHLKELPSNLHKLTDLHRLELMYTGVRKVPAHLGKLEYLQVLMSSFNVGKSREFSIQQLGELNLHGSLSIENLQNVENPSDALAVDLKNKTHLVELELEWDSDWNPDDSTKERDEIVIENLQPSKHLEKLTMRNYGGKQFPSWLSDNSSLNVVSLSLRNCQSCQRLPPLGLLPFLKELSIEGLDGIVSINADFFGSSSCSFTSLESLRFSNMKEWEEWECKGVTGAFPRLQRLSIGYCPKLKGLPPLGLLPFLKELSIEGLDGIVSINADFFGSSSCSFTSLESLKFSDMKEWEEWECKGVTGAFPRLQRLSIRYCPKLKGLPPLGLLPFLKELSIQRLDGIVSINADFFGSSSCSFTSLESLDFYDMKEWEEWECKGVTGAFPRLQRLSIYNCPKLKWHLPEQLSHLNRLGISGWDSLTTIPLDIFPILRELDIRECLNLQGISQGQTHNHLQRLSMRECPQLESLPEGMHVLLPSLDYLGIIRCPKVEMFPEGGLPSNLKNMHLYGSYKLMSSLKSALGGNHSLETLRIGGVDVECLPEEDISHCEDLKRLDYKGLCHLSSLKELTLWNCRRLQCLPEEGLPKSISTLTIRRCGFLKQRCREPQGEDWPKIAHIEDVDIRCFSIACNLLINYSFNSSIFFNKID

SEQ ID NO: 64 - DNA, 3885 bp, glycine max
atggcactagaattggtcggtggtgctcttctttctgctttccttcaggctgcgtttcagaagcttgcttctcatcaaattcgggacttctttcgtggaagaaagcttgatcaaaagctgctcaacaacttggaaatcaagctgaactccatacaggctctggctgatgatgcagaactaaagcagttcagagatccacgtgtcaggaactggcttcttaaggtcaaagatgctgtgtttgacgcagaggatctcttggatgaaatacaacatgagatctccaaatgccaggtcgacgctgaagctgaagctgagtctcaaacctgcacttgcaaggtaccaaatttcttcaaatcttctcctgttagttccttttacaaggaaattaagtcaaggatggaacaggtccttgaggaccttgaaaatcttgctagccaaagtggctatctaggtttgaaaaatgcaagtggtgttggatcaggatttggtggtgcagtatcacagcaatcacaatcaacatctttgcttgttgaacgtgttatttatggcagagatgatgacaaagaaatgatctttaattggctcacatctgacattgacaattgtaacaaaccatcaatattttctattgtgggcatgggtgggttgggtaagaccacacttgcccaacatgtattcaatgacccaaggatagaaaataaatttgatatcaaagcttgggtctgtgtttcagatgaatttgatgttttcaatgtaacaagaacaattcttgaggcagtcactaagtccactgatgatagtagaaaccgagaaatggttcagggaaggttgaaagaaaaattgacaggaaagagattttttcttgttttggatgatgtttggaacagaaatcaaaaggaatgggaagctttgcaaactcctcttaatgatggggctccagggagtaaaattgttgtcactacacgtgacaagaaagttgcttcaatagtgggatcaaataaaacacattgcttagagctgttgcaagatgatcattgctggcaattgttggctaaacatgcattccaagatgatagtcatcaaccaaatgcagacttcaaggagattggtacgaagatagtagcaaaatgtaaaggactgcctctggctttgacaacaattggaagtctattacaccaaaaatcatctatttcagaatgggaaggcatattgaaaagcgagatatgggaattttcagaagaggatagctctattgtccctgctttagcactaagctatcaccacctcccttcccgtctcaagagatgttttgcttattgcgccttattccccaaagattacaggtttggcaaggagggtttaattcagttgtggatggcggaaaactttttacaatgtcatcaacagagtaggagtccagaagaagtcggtgaacaatacttcaacgatcttttgtcaaggtccttctttcaacaatcaagcaacatagagggaaaaccttttgtcatgcatgaccttctcaatgatttggcaaaatatgtttgtggggacttctgcttcaggttggaagatgatcaaccaaaacatataccaaagacaacccgtcatttttcagttgcaagcaatcacgttaaatgctttgatgggtttggaactttatacaatgctgaaaggttacgcacatttatgtcattaagtgaggaaacgagttttcataactacagtcgttggtattgcaagatgtcaacacgtgaattattctccaagtttaagttcttacgtgtcttatctgtgtctgattattctaacctaacagagttgcctgactccgtaggtaatcttaaatatctccattccttagacctctcaaatactggcatagaaaaactacctgaatcaacatgttcactctacaacttgcaaatactgaagctaaatggttgtaaacatttgaaggagctgccctcaaatttgcataaactcactgatttgcatcgccttgaattaatgtatactggagtgagaaaggtaccagcacatttgggaaaactggagtatcttcaagtattaatgagttcgtttaatgttggcaaaagtagggagttcagtatccagcagctaggagaactcaatcttcatggaagtctatcaattgagaacctgcagaatgttgagaatccctctgatgcattggctgtggatttaaaaaataaaacacaccttgtcgagctagagttagaatgggattcagactggaaccccgatgattcaacaaaagaaagggatgaaattgtaatcgagaatctacagccttccaaacacttggagaagttgacgatgaggaactatgggggtaaacaatttcctagttggttatcggataattcatcattgaatgtggtgtccttaagcttgaggaactgtcaatcttgccaacgtttgcctccccttggacttttgccatttctgaaggagctttcaattgaagggcttgatgggattgtgagtattaatgctgattttttcgggagtagctcttgttcatttacatccttggaatctttgaggttctccaatatgaaggaatgggaagaatgggaatgtaaaggtgtgacaggtgcttttccacgtcttcaacgtctttctatagggtattgtcccaagctgaaaggtttgcctccccttggacttttgccatttctgaaggagctttcaattgaagggcttgatgggattgtgagtattaatgctgattttttcgggagtagctcttgttcatttacatccttggaatctttgaagttctccgatatgaaggaatgggaagaatgggaatgtaaaggtgtgacaggtgcttttccacgtcttcaacgtctttctataaggtattgtcccaagctgaaaggtttgcctccccttggacttttgccatttctgaaggagctttcaattcaaaggcttgatgggattgtgagtattaatgctgattttttcgggagtagctcttgttcatttacatccttggaatctttggatttctacgatatgaaggaatgggaagaatgggaatgtaaaggtgtgacaggtgcttttccacgtcttcaacgtctttctatatataattgtcccaagctgaaatggcacttgccagagcaactatctcatttaaatcgtctagggatttctggatgggactctctaacgaccattccgctagatatcttcccaatactcagggagcttgatatcagggagtgtcttaatctacaggggatttcacaggggcagactcataatcatctccagcgtctgagtatgagagagtgcccccaattagaatcattgcctgaaggaatgcatgtcctccttccatctcttgattatctgggcataattcgttgtccaaaagttgaaatgtttcccgaaggaggtttgccatcaaatttaaaaaatatgcatctctatggtagttacaaacttatgtcctcattgaaaagtgccttgggaggcaatcactctctagaaaccttacggattggaggagtggatgttgagtgtcttcctgaggaagacatcagtcattgtgaagatctaaaaagactggactacaaaggtctctgccacctctcctctctcaaggaattgactctttggaactgccgcaggctccaatgcttaccagaggagggtctgcccaaatccatttcaactttgacaattcggaggtgtgggtttctcaaacaacgttgccgggaaccccaaggcgaagactggccaaagattgctcacattgaagacgtggatataagatgcttctctattgcctgcaatttactaattaattactctttcaattcatccatattctttaataaaattgattaa

SEQ ID NO: 65 - DNA, 48740 bp, glycine max
acgtcaagtgatgacttaacacttgttttattatcattttattttatttcgttttctaatgatttactttatatttttctctttttttctttatattcaagttttgtccttcctacttcttctcccttatcaacaattcttccccaagacaaccctgccgttactactaccctttctcctcccctctcctctacaatcgtggtcgtggctgcttcactaaaaacactccctcaacatgacaagttttttgagagttattactaatgtcatgtctcccttattgttaacgtcgtcctcatctttgcttcttacctcatgaaacttcgttttttttttttatctttctctttaatttctcccacttcccaccaaaatggtagggatctggagcaagtttagtttttgtagtttgttcttctaggttgttggcaacaacaggtagcaagtgccctccatgattttggtgacaatgaggttttgaatattgggttttttgtgtgcaaggaattgtcgtgttgctatcgtaattcaattgtgatgaaccccatgaatcatatgaccgtgatagggaacttgtgagaggtggttgagggatcaaatgggtttgaaaacagaaattgtgagttctatttcgtgataatgatgaaggttgacgacggtttaaagatctgcttaggatttaaagattcactatccatggtcatgggtttgactttacccactaaaaattaaatttttgttaaatttgaagaatgaaacttccctccgtttccattaaatttgaagaatgaagcttaactccaagtttgttgacctaatttgtgtttccatcttaatttgaaattgtgcatatgatgaatttggatttaggctaataagttttgtgcttgaatttaaaattggaatatagcaatctttgacggtttttaatgcaagaatatgtttaataatttttaaaccaattattcatgtaatttatggcaattttagtggctagtaaacctaataaaataaaataaaataaaaatcaaataaaatggttatgccaccaattagcatctaaaatagtaaaaatgaagaaaaccatggataaaataaaagttgaaggactaagatcaatttcaaaagggtcaaggatgaaaactaaaaatttgaaaaagataaaggacaaaaacatagtttgctctaaagaaaaatttatatgaaccaaaatcaaaacatgatcatcttagaggaactaaaagcatatttatatttatgcctaccagcatatataagaaaagagcaatgggtgaaagtgtccaaccatcaaaaccagcatttcatatatcagcaatgcaatgggcctttatagtcgaagacccttcttcaaaaggccgaaggttttcaatagggattacataaattcttccatcaaagccaattgaacaaccatactagatatattaattaaggatgtgctcaaagaacagaaaattctatgttccatttctaatatctgagtagattcttatgatcatctactacttgtcatttactttatacataaaagataaactgcttcgtcaaaaaaaagaaacaaaaacaaaactgcccttattataacaaaatgtggcaatttcaatctttggtgagcaaagtgcaccaatctagggaattaagtacagataggtagggtttttgcctttcaaaactgccattatttttttttctttttatgatgcagttacttatttattttttatttgataccatcatattttctgcttcccctgttcttcattcataccaaacaaactaataaaaaaaaccaaatctaggacgtgaagagttaaaatcacataaattatggtccattataccaagtacacaacacgagtacaattgcaatatgcaagctaaaagagtaatgttatattttttcatataaaaatcttcctattaatggaccatttttaaacttgagtttctcaactcactaggtttaaaattgagttttcaatgttcttaatccatttttgtgtaaaagcattgtttggtgggttttctgttgacgtacctagattgattatttaagacctagatggatcattgatgctagtcctaatgatactgagaagtttacaagacatacctaagacaatactttttgttctttaatattcaattttgagccttgacttcttaacgtctttcttctattaatttttggctttggtttgaattatgttgagaatgtcttaattgttggctttgtcttcatcaaaattcaccattagaatttttaataaataatcaattttgtccttatttttgtagttattttacaaattaatttttatcctattgaaatgtaaaaaatagttattttctttagatttacaaataagtctttaatgttaaaatttaattttgactattagtcatgtatctatatgacaagtctcatgtaagcaaactcatgtgttgatgtaaacaaacgcgtgtaagaagtctttgtactgaattgaaagccccttttcttttacatctcttctctctcccccacttctttccccctcttcttctatctgcccccatcgtgtaccaccatatctccaccgcacgcaaccgcgccgtgttgccgccaccacctatcccactatgctgcctgccactgaccaaggcttctgcatttccatccatctccacactggtttctgagaccaacgaactgaggagaacgcgtggtgtgaaggtcgcagcgacgatcctcgaagacggcgagcactgagacttgttggtgcggagaagccgtggaaaactctatttattgagtcatttattttgttagacaggagatcgaaagacaaaaatcctaacaacatggaggtgccgtgattggctcctggaggaagcggagaaggcgtcacagtgagggttttgcaagtgcgaacgtgatgatgatgtcgaggctccctatgagtgtagcggagaggtttgttgcaagcttgagtgcagcggcatcgagattccctccgggcgtgaacacgatggcgttgaggctcctgcaagcgcggcggcgaaatgtcgtgccgtcgtttcgctaccgagaaacgaatagatattttttttatttgttcgatttggatttgttttctgaatctgattatttctccaatttattttatctgctatataatttttttttggatctgccatggtttgttctttggggtctgctatgtaattgattttcgggtcctctttttttttggatcctctgtgtaattgattttcacttctgccaatgttgttcttttggatccacttttttggtctgctttatagtgcattgtttttaaactgccattgttgttcttaaaattagaagaatgcatttgaagaaggttgttctttggtagtggaggaagaattgcagtggtctgccggtatcctgcatgtttggaagcggtggaggtgttgctgttgacggcgaccatgtcgacggaagttggaagaatgaaatgagggaggaatacctaaactttagtgtcacgttggtaacaatacatctcactaatgggttattttcaaatttaacaataagaactattttacaacattataaattataaagatagagattgttttttatattttaatagaatagagactaatttgcaaatgaccagatgcctagaatttccttggataatcttcttgagtgttgggatgttctacagttagggagtcattttttttttgtttcttttttatgcttacacccgtgatagtgcttccggggcgtttaagtgctactctaaagcccaggtgaaatttgttgcagctgatgcttttgggccttcctcgtatgattgacctcctgctctcacatttgtattctatcacatggacgtttcattttcacagggataacgagagctaacaaagaatctaattaatcatgaatcaagtccttaaaattttaaggtgtgttttctcttttatgttctatcgtaccttggacatcaagtacaacatttctttgccatattttttcatgggtggatttttaacttgttattggtgaattgttatctatggtataataataataataatacaaaaacaattaatgtaaaccataacaaataatataatagaataatggttgtatcactatccattctctcactttttgcgtggatcgctctgctcagctatcctagattgataattatacttgtagcattcggaatcaatgttatgaatgattaggaatactaaggaagctgcccatagaagacctctaacctagcccagcatggtagagtaacttaattggtaggtatcctcactggtcactgtttatttatcatcatcattgttgttttgatcatttcataatcataatcaatctaaaacagatccaaaattcctttcaattaaccacatgcactaaaatcttaactatagttgactacttcttgtaggtgcgggtgcgtatgacactatagagaagagtaggtgcttcaactctataataatagcacttatgttgagctgcaagtgcaaaaggtacggtcattgcctttaagatctgaaatctcataccttcttcaatgtggtttaaacaatgttatgctgttttccatattcaaaattatttaatttcacataattaacgtagaatgagaaaaataagtccccagctgggtaaataattaaaataaattttgatcaatgtgttttagattgaaagtaaattaatgatggtaatctttataaacacaagcaaattagtatattttcagttgaaaattaatttatcaacatatatttgatgttttttttattaataatacaaatatatcgaagtagaagccgacgtgccgttgacagtgagacccagatcggatcttactttatgtttaatcaagaaattcatactgtccgtttgtgactaatttatcaaattcaattaaagaaaaaaaaaatcagtgtcttactttgtacatttcaccatatacaatgaagtgtatcatttaggtaaatgataatttgcttgtattatttacgtaaataattgattaatttgtatttttaatgtaaagactcccaaatcaatagaaaattctgaacatgaaatatgtgtatgaaagattttaacgtgattaaaaatatgtttaaaataatataagtggaatcaaggctgatcttataagtatgtatttgaggtttagatagatttaaatccaaattttaacaacatttaaagttaatgaagtgatattttaattttaatttaagaataatgccttcactacttaaagaattgaacctaacttttgtccatttggtaataaccaagctctattccctttgattttttttattcatttttaagtgttctgattttgattttctggtatgaatgaattttcttggagctattaagtgttttttgtgggaacgtaaagcctgaatttcaaacactaaacaaaaattataggttgcattaagttgagaaataccctgcgagctcttgaatttgaacgataattctgaaagatgaagattaagagaaatttgggacaacttcttgagtttctcaaaagcttttgatgaatcagatgcatgtgagttttctagaatggttgtttggtggaattttcttattaaattgagtttaactgcaacctttgaagatgaaacgtttaagtaataaggatttgtaaagtttacgaggcatgcaattctctttactggttggaaattgcaatcagctaacttgaagaatcaagctgctagattttaaagttggtatgggatattttgattgaaccatgttgatacttgacaactagcttgaacaattttctttgttgaattattgtagtgcacagaactttaataatgtctaactattattattgttaggcattatagaggatgaaggggtgtcggtacaattaaagttccaccaaactttgtctgttaatgaaattctcccactatttgagtattatttttttaagggtatatttgataaaattagctaaaatattaataaataattaaaagttaaaaaactaatttattaaattaaaagtgtttaataatattagttattgaaataatcataaaaaataacagaaaaataataaaatctagattcatttaaaaaacataacaacaaaaataagtaaatatatcaaagataaaaataaaaaattaaaagtaagcaatttaaaaaatactagttaagcaacatgtctaaaaatattaaaaattattaaaaaaactcatttatcaaaccatcaaataaatttttaattaataaaaaaaaataaaatatcttactaaacataacttaaattattttttatcaattaatactatcttttatatatgcttaagcattcttatgagttctcaaattttcaaatatctcatcattatctctttatatatatatatatatatatatatatatatatatatatatatatatatatatatatatatatatatatatacttgttatattatatcatcaattttattcatcttttatattcatctctttttattatttctctccagctgtttatcctttaggtgtccaaacataattttccttttagttaatatttgtatttaaaattaatttgttactaatttaaaaatgtgatccataactaacaacgcttgattaagttattaaaaatcaaactcatatttcagaaaaatgtaagtactttataaatatttttataaacattcagtgagccataaaatatgttttgatgtcagtaaattctcaagatctacctcatttaaattttagtaataagaaaaaatatatgatgcatgaaatgaaaaaatgagagttatatatttgatggggtttatttatatagtattaaataaatttcacttaataaaaaaggttctctgccgccacatttgaatgtgtacacatattaccattttattacaacaacagggggaggggtcctcatctcatgcctgtgaagtttctttaagaacaagtggagaaaagttcaaactttttacgtaaaattgagttttttttaaaattatttgcaatattttatgatatgagaggcattgaattccagaaccacatcacacgcttccaagcacgctttccattattatactaaattattttttaacaattaatttattcattcaattccttaagataatttcttcctttataactatttaaataactaatttttaataaataataatttttgtatactatttttactcataaaattaaaacaaacaagtgttggttaatatcccaagaggtaccctcagcagaatcatggccacgcattaattcatgaaatacagctgttaaagtttgtcttgttgggaagactcaacagatattggttgactcaatcttaattaaagccatccttcataaaattgcctatcactgatttgatcagttcatgaaccctgcacttcacttcaaattactcattttcattcatccactcaactccctttcctaccatggcactagaattggttggtggtgctcttctttctgctttccttcaggttgcgtttgagaagcttgcttctcctcaagttctggacttctttcgtggaagaaagcttgatgaaaagctgctcaacaacttggaaatcaagctgaactccatacaggctctggctgatgatgcagaactaaagcagttcagagatccacctgtcaggaactggcttcttaaggtcaaagatgctttgtttgacgcagaggatctcttggatgaaatacaacatgagatctccaaatgccaggtcgaagctgaagctgaagctgagtctcaaacctgcacttgcaaggtaccaaatttcttcaaatcttctcctgttggttcctttaacaaggaaattaaatcaaggatggaacaggtccttgaggaccttgaaaatcttgctagccaaagtggctatctaggtttgcaaaatgcaagtggtgttggatcaggatttggtggtgcagtatcactgcattcagaatcaacatctttggttgttgaaagtgttatttatggcagagatgatgacaaagaaatgatctttaattggctcacatctgacattgacaattgtaacaaactatcaatactttctattgtgggcatgggtgggttgggtaagaccacacttgcccaacatgtattcaatgacccaaggatagaaaataaatttgatatcaaagcttgggtctgtgtttcagatgaatttgatgttttcaatgtaacaagaacaattctggaggcagtcactaaatcaactgatgatagtagaaaccgagaaacggttcagggaaggttgagagaaaaattgacaggaaataaattttttcttgttttggatgatgtttggaacagaaatcaaaaggaatggaaagatttgcaaactcctcttaattatggggcttcagggagtaaaattgttgtcactacacgtgacaagaaagttgcttcaatagtgggatcaaataaaacacattgcttagagctgttgcaagatgatcattgctggcgattgttcactaaacatgcattccgagatgatagtcatcaaccaaatccagacttcaaggagattggtacgaagatagtagaaaaatgtaaaggactgcctctggccttgacaacaattggaagtctattacaccaaaaatcatctatttcagaatgggaaggcatattgaaaagcgagatatgggaattttcagaagaggatagctctattgtccctgctttagcactaagctatcaccaccttccttcccatctcaagagatgttttgcttattgcgccttattccccaaagattacaggtttgacaaggagggtttaattcagctgtggatggcggaaaactttttacaatgtcatcaacagagtaggagtccagaaaaagtcggtgaacaatacttcaacgatcttttgtcaaggtccttatttcaacaatcaagcaccgtagagagaacaccttttgtcatgcatgaccttctcaatgatttggcaaaatatgtttgtggggacatctgcttcaggttggaaaatgatcaagcaacaaatataccgaagacaacccgtcatttttcagttgcaagcgatcacgttacatgctttgatgggtttcgaactttatacaatgctgaaaggttacgcacatttatgtcattaagtgaggaaatgagttttcgtaactacaatcgttggtattgcaagatgtcaacacgtgaattattctccaagtttaagttcttacgtgtcttatctctgtctggttattctaacctaacagagttgcctgactctgtaggtaatcttaaatatctccattccttagacctctcaaatactggcataaaaaaactacctgaatcaacatgttcactctacaacttgcaaatactgaagctaaatggttgtaaacatttgaaggagctgccctcaaatttgcataaactcactgatttgcatcgccttgaattaatgtatactggagtgagaaaggtaccagcacatttgggaaaactggagtatcttcaagtattaatgagttcgtttaatgttggcaaaagtagggagttcagtatccagcagctaggagaactcaatcttcatggaagtctatcaataaggcaactgcagaatgttgagaatccatcagatgcattagcggtggatttaaaaaataaaacacaccttgtcgagctagagttagaatgggattcagactggaaccctgatgattcaacaaaagaaagggatgtaatcgagaatctacagccttccaaacacttgaagaagttgacgatgaggaactacaggggtaaacaatttcctagttggttattggataattcatcatgtaatgtggtgtccttaagcttgaataactgtaaaggttgcctatgtttgcctccccttggacgtttgccatctctgaaggagctttatattgaagggcttgatgggattgtgagtattaatgctgattttttggggagtagctcttgttcatttacatccttggaatctttggagttctccgatatgaaggaatgggaagaatgggaatgtaaaggtgtgacaggtgcttttccacgtcttcgacgtctttctatagagcgttgtcccaagctgaaagggcacttgccagagcaactatgtcatttaaattctctaaagatttctgggtgggactctctaacgactattccgctagatatcttcccaatactcaaggagcttcagatctgggagtgtcctaatctacagaggatttcacaggggcaggctcttaatcatctcgagactctgagaatgagagagtgcccccaattagaatcattgcctgaaggaatgcatgtcctccttccatctcttgattctctgtggatagatgattgtccaaaagttgaaatgtttcccgaaggaggtttgccatcaaatttaaaaagtatgggtctctatggtggtagttacaaacttatctccttattgaaaagtgccttgggaggcaatcactctctagaaaccttagatattggaagagtggatgttgagtgtcttcctgacgaaggtgtactgccacactctcttcttactctagtgatcaaggactgtggagatctaaaaagactggactacaaaggtctctgccacctctcctctctcaagacattgactctttggggctgccccaggctccaatgcttaccagaggagggtctgcccaaatccatttcaactttgggaattctcaattgtccgttgctcaaacaacgttgccgggaacccgaaggcgaagactggccaaagattgctcacattaaacgcgtgtggttatatggaagtgaggttgattataaagaataatggtgaaagatgtagatgcttctctattgcctgcaatttactaaataattgttttctaaacttatttttgctttctgcttgctggacgccgccgctcagttatttattattcgacaaaataattgtttaatatagtttcaagtgattgattctgcttggataaaatatttcactcttgaattttactactaattttttcgataaattattatgttatccacattatattttgacatattgttaattaacttgaagagttttaagttctaaaaagatgataggtttaatataattaaaggtgatttcaattcctacctgttgagtaatatgaaattcaatttattacattaggtgtcatttatataaaaaaatttcttttaaaaaagatcaaaatggaagtttaaaaataaattagagaactaaaataataattaaacctataatttattactcttatcctgtaataagactctttcaattcatccatattctttaacaaaattgattaatttagttagtaatattaaatttattagtattttatattatctttttaaattatctttaaaattattgagactatcatcaatctttttttcacttaattactttttggctaattaatgaatatgtgttaatcttcttctttgatccttataagagggaatgtatttatctttttaattgatatttattataaaataattaagagtattttgataaaaaaaaataattaatgtgaaagagaatctaaaaatagtattataaaaaagaaaaatatcttattgacggaggtagtaattgaagcgtacatacaaatttatgttaaaattaaaataatacttcaatcaattactatataatcatttataaataaaaaataattataatatatatatatatatatatatatatatatatattcaaattatatttttaattaattttatataattaattaacttctttataaaaaaaacttaaaaaaacactataattaattaattacataaaaaataatatataataattataaataaaaataaaacaataaatttaaaaaaaaaatgaaaataatctatttttttattaaaatatattaactatttcaaaacaacattctaaataacatattataattaaaaaatacaataaatatttaaaaacatacatcaaaataatctctttttatttaaaaaaacaaacaatagctatttaaaaataaaataaaataaatatatttgcaattgaaaaataactatttaaaaacataattcaaaataaactatttttatttaaaaaagaaacaatagctatttaaaaaataaaattaatatatttacaattagaaaagattaactatttaaaaacataattcaaaataaaatattttttgatttaaaaaacaatagctatttaaaaaataaaattaatatatttacaattaaaaaattaactatttaaaaacataattcaaaataaactatttttatttaaaaaaaaaagaaaaaatagatattgaaaaaaaaattaatatatgtacaattaaaaaattaaatatttaaaaacataattcaaaataaactatttttattttaaaaaatatatatatatttaaaaacaaaaataaaattaagatatttacaatataaaaaattaactattaaaaatcataattcaaaataaaatattcttattttaaaaaagaaacaataaatatttaaaaacaataataaaattaatatacttacaattaaaaaaattaccatttaaaaacataattcaaaattatctatttttatttaaaaagtaaacaataactatttacaaacagaaaacaaaataatatatttatatttaataaaaacaataacaatttagaaacaaaaatcaaaataacatttttataaataaagaacatttactatcaaaaaaattaatttattaataattaaaaaaaacatacaaattacataaaatacgaatttatgtaatccatgtgttttttttaattattaatccatccggattagttaatctgtataattcatatggattacttaatctgtatgtgttatacggatttcaaagaagataaaacaaccattaaagaagggaagaagaaaccttacctcgacgatggaaacctcctcaaagctagcaatggaagcagtcaacggcggcagcacaacacaaaacaaagaagaggatgcaagaagacgaaggaagaagaagtggcgtacggacaaagaataggagggaagctttttaaaatttaagtgaaagacatttttgttacttcacttaaattattaatgcacaagcaatattacagggtgtatctagcaatatcgttcctgtatataagcaattgaaaggggaagaggaggggttacccagaaaccaaacatctaagacttgccaaagattgctcacattgaatttcttggagatttcaccatggtaatggtgttgatttttttaaccatggtaatccaattagcattatttaggggggaaatgggtttgtctgaccaaactaatattcaatacggcaaagtaaatttttgcattagttgaattttccgtttgccatttttttgtaataaaactggtatatgataattggttataaaaatgagtcaaattatttcttaactttaacgttcaaatagaaaattttatgctatatgaccatgctgcggtgaagaactcactaaactcattctaaatgaagtgtttataagttgtatccgatcaatatattttcaatcgatgtcatcgtattggtagacatgacataagggataaaatgtttgaagtttagattgggcacattagagtcattatgatgaatttaaaaggagattggatgttcggagtgtaagaatttaggactcttggattttatggtttggatgataaaagtatttttagtctttactattaacttcttagcaaaaggtcaagggtaacactcactagtatctatggtataatattactatatgatgcttaatttttttttctaaatttctaaaataaatgctatttatctacttcaaaattaacttttaagatcaaaaataagaattctagattttagaaacaacaaaatcttatatcatgagatgaagtcagttacatgaatcacataaccttatttgacacaattgaaaattgaaatttcagaaattaatacattattaaatcttatgtctcccttgatgagttactctaatgtttttttcaatttctctttttcttaagctaaaaatcataacatctactattctaactgatacctcttgttgtcttttttgtaaatggccaaagattttgaaaatgttagaagaaaaatatttgagagacgttttttttactcattttaaaggaatggtaaataattaattttaaattttagagggatgataaataatgaaaaagagcaaactataatttttgtactttaactttttaaaaaaatttatttttgtccctgaactttttgaagactaatcatagtcctccaacttgtattccgtttatgatttctgtctttttggatgttaactaattaatgagtgacacttgatttattattattattattgttgttttattttattacatttcctgacaactaaaaaccatcagaaattattattattttctttattttacaaatttgtctcttgtctttcttctccctcatgtacaattcttccccagcacactcccactgtcactactaccccttccacctccacccaccatcaccgttacttccctgaaaatgctctgtcaatccaacaagtttcttgaataagaatgtttatggatccaggtctatccttgacccaacccgatccaattctgttgggttggtttttttaagtgtttatgttgaattcgacccaactcaattaagtcttgattatatacaaattggataacaagatctattttttaacttaattcaactatacctaactcatatcatataattaaatttattattatatataaattacttattaaatacaaaacacattaatttttagttcacataatttattaaattaaatcacccatgaattttttctttaagtaaatatttttataattgtcttgattttgtttatgtttgtcatgctaatacaatattttatttatatctaaaataaaaatatcgtattagagttgaaatagttaactctattagtcaaatattgtctcttttgaatgcatttagtcattatatacttacaaagttttaggaaaaacaaattattattctaaaaaaatttagtttttgcttttgtatttttttgtggttttgttcttttggtcaattgtcgaagtgggtgttggcaacaacggggttttagataatgagttttatttatttattttgtgcgtgtgtgcaaaggggttttagataatgagttttatttatttattttgtgcgtgtgtggaaaagagccgtcatgttattgttttagtttgaggtggttgagggagcaaacaaggtgcaatagaggaattgcaagatctatttgatggtgacgatgaaggttgatggtggtttgaaaatttgtaaacagggtggtttgaagacttgcgagatctgcgtagggtttgaagatttagtgtccatggccatggatttgactttacccattaaaaattgaatcttggctaaatttgaagaatgaagcttacccgtttccatgaaatttgaagaatgaagcttacctttgagtttgttgatctgatttgtgtttcaatcttaatttgaaattgtgcatatcatcaattttgatttaggctaatcatttttgtgcttgaattaaaatttgagatatagcaatttgtgaaggcttttagctgctagaatatgttcaagacttttcaaataagttattcaagtaatttttggcaatttttagccaccaagaaataaaataaaataataataaacaaatattaggacaccatttaatgtctaaaataaatagaaatgatgaaaactgtgaatgtaataaaagttgaaagattaagaacagttttcaaaaagtttcaggatgaaagacaaaaatttgaaaaagttaaaggatgaaaaacatagtttgctctagaaaaaaatttataagaaccaaaatcaaaacatactcattttatagaaactaaaaatatagttaagcctaagagtatatataaaagagtaataggtagttaagcctaagagtatatataaaagagtaataggtgaatgtgccaaccatcaaaccaccatttcatatatcaacaatgtgatggacctttatagaagacccttcttcaaaaggacaaatgttttcaataggattacacaaattcttccatctaacggaaattctatgttccatttctaatctgagtagatttttgtgatcatctactacttttcattgatttcatgcataaaagataaactgctctgtcaaaaaaaagaagataaactgcccttataagcaaaatgcacctgtgtagggagtaaactacagataggtagggttttgccgttcaaaattaaacacataaagccaagttgcaaaagattgtagcttccatgacagaatgctttgcttcatatcatttattccagaaaggtccaaattttttaagtggcattatctacatgcaaaaatcaaaaggtcaggggaagttgacagaagaaaaaaggagggaaaaggaagcagaggcttgggtggtagtgagtcatcatatcatatatacacaaactttccagaacatcttgccatcaataaggtcaaaaggaacaggaccaaattttctaaataattgttttctaaacttatttttgctttccgctgagttatttattattggacaaaataattgtttaatatagtttaaacgccgcgctgagttatttattattggacaaaataattgtttaatatagtttaaagtgattttagtccctggatattgtgtaatacggaattctagccacattggtgcatgctgctgtgaattaagagcagctaatgatttgtaggcatcctgttcagcgtttcacctgttttgtagacttatattatcattggttgatgctgttcatgctttctagtatttgttacttttttcattagttatattgtttgttatcgtcgtgagttcaaaggatcaatcacaaatgggatgaaattttaattggatttatgaaatctaatttatgtagttactacttttttttctcctttttatgatgcagtgatttattttttatttgataccaacataaataacaccttaagctccccggcaatatctcaaatatttttataagcttcaatggatcgactattgagctaattcttggtcggagtaacactatctaatattttgaattaggactttatctctatattattatgaagtattcgtatgtttaaggccaatgtcgcaaataatttgttagcatgagaaaatgttttctatatgaagatgagatagttataaaatcaacgggttactcctctagacaataaagtcgaagtaatatttctcttaatgtaattaaacattatttaggggaaaaatgaatttatttggtcacactaaatgaaatgtttaagtaatacaggtttgtaaaatttgatgactgtaaatacaaatctatgaggcatgtaattctctttagtggttgcaaattactatacgctaactacaaggcttaagctgatagattttgaagtcttttttttcctgattgaaacaagttgatactcaataacttgcctgaacaatttttctttgttgaattttgcagtgtgtagaactttatagtctattattatgcattataaatgttatttgattttaatggtcaactggtcattggtttatttattttagaggatgaacgggttgcctttttagtctattgtgattgttttccctttaaacatctcttttcatatatcatgctgttatatccttttgtttgacatttctttcaggctatgactcagttagttgctaaatcatgtgaagatgcccggcaaaaggtttgttgtggcttcggctttccttaacttttttgtaaggtttcagttagcaatttaagggaataataatagtgtagttgtggaaagaatgaaattctccctcgctctgtgaatattatccgcttttttaacttgaattatttttatagtatcctaaaagaattgaaatttatagagttactgtagagtttcatgtgaaagtcaaaagtcttttaagtgatcataaaaataaataaaaaatataacataaaggaacaataaaaactatagaaaaatacattcttttactttattgtaagtgaacacttttatcatcaatttgatataaataaaatcaattatgattagtgtaagcggtagtgacttgtgtctcttaattaaatgattaatgatttaaattttatttggcatttaggtatgaaataaattgtgttgcgagaaaaatataacttttatatctacatatattgttgttataatattaacataatgacttcattaattcaaaacctccatgatgttggtgacaatgagattttgaataatgaatttttttttttgcaaagaaatcatcatgttgctatcgtaattcaattgtgatgaatcccatgaatcatatgaccatgataaggaacttgtgaaaggtgattgaggaatcaaatggattggaaaagaggaattgtaatatccatttttttggtattgatgaaggttaacaatggtttaagaatctacttaagatttaaagactccttgtccatgatggatttgactttaaccactaaaaattaaatcttccttaaatttgaagaatgaagcttaactccaagtttgttgacctattttgtgtttccatcttaatttgaaattgtgcatgtgatgaatttagatttatgctaataatttttgtgcttgaatttaaaattggaatatagcaatctttgacggtttttaatgcaagaatatgtctaagactttttaaataaattattcatgtaatttatggcaatttttagtggctagaaaacatactagaataaaataataatcaaagaaaatggtcatgtcaccaattagcatctaaaaaatagacaaaaatgaagaaaaccatgaacgaaataaaagttgaaggactaagatcaatctcaaaagagtcaggaatgaaaactagaaatttgaagaagataaaggacgaaaacatagtttgctctaaaaaaaattacatgaaccaaaatcaaaacatgataatcttagaggaactaaaagcatatttatccctaccagtatatataagaaaagagcaatgggtgaaagtgtccaaccatcaaaccagcatttcatatatcagcaatgcaatgggcctttatagtccaagaccctacttcaaaaggccataggttttcaataggattacataaattcttccatcaaagccaattgaacaaccatactagatatattaaggatgtgctcaaacaacagaaaattctatgagtagattcttatgatcatctactacttgtcatttacttcatgcataaaagataaactgcttcgtcaaaaaaaaaaaaaacctgcccttataaaaaaatgtggcaatttgaatctttggtgagcaaagtgcaccagtctagggaattaactacagataggtagggttttgcctttcaaaattaaacatacaaagccatgctgcaaaagattgtagcttccatgacagaatgctttgcttcagatcatttattccagaaaggtccaaattttttaagtggcgttatctacatccaaaaattaaaagatcaggggaagttcacagaagaaaaaaggaagcagaggcttgtgtggtgagtcatcatatcatacacaaacttaccctccagaacatcttgccatcaataaggtcataaggaacaggaccaaattttcttgaatcattgctgttatacttgttatctccctcaacccaaacggcaccctttggcacctatagagggaaggcatacttagacaatgaaatctccaaaacacattcagcgatggaaaaaattaattatggtcaaaattctctagaccaaaggccatcaaatatcaagccaattcttaatgcgaagaagcaaggatgccagatatcagtcatcccaaaaataacaatcaaaataccagcaaagcaaaacccataatcaaaatgatcacaaaaaatgggaatttaattttcataagaatttgaccataccacaatcgtcttagacttatcaccgttatcaggagaggaaatgcgtgtaaagctatcaccctcatattcataggtctcaggattggaaatgtatgtaacgctatcaccctccaatccaaccacacgtttggtcataaagtgcctagggtgttgaggattgcgcaagactacaatatctccacaagcaactttaccaaaccggggtgagatcttttccattaagaacacacctgttttcaaatcgatagtaggaagcatgctaggaccatagatctgcagcagcactagaagtggttacttgataaaatagaatttaagacaggttgacaaagaaatcatgcttaccacagcaggggcgatcaaataggtttgggtaacgtgtataaaacagtagatcttagaaagcctccatcctgctttaataataggcacaaatgcctccaggcttctcaatatcatcacttccgttttcctctgcaggtacaaatcccaaatactgttatcctaagtggaaaagaaaacaagtgctctgctcactattagcaaacctaaaacatgtcagatgtgagtgttagtatgaagaaggacgaatagcaagggttttgcagcagatgaagtttgggtcagagataatggtggtgtaggtggatcaaaaggtgtcagataaataagatgatgggagaaaagaaaattgagaaaaagaataaataaaaaaaaaaaagcatgtatgtttattgttagctcattatataacacagaagtagcactattacattattttctacttgacaaaacaaattcaactttttctcaatttttatgacaatttatcaatcaaacagcatcaagaaagaagttgtatatctcttggtgaagcaaaagtttattcgcgggtaaagtatataatatcaatgtacctttccataaaatcaactctcacgactaatttaataacaactactataggcaaaagtgagataaaaagaaaatacaaactgaagtagattacaacaaaaggaagaaaaacttccttctacaaaacaaccctgcagcaagtaataacccaattgccattataagcatttgatgctaacaaatttagatgccagagttaaacaaatagtagtaaatcaaacactgcaaaattcattcatcaagtgtctgtttggaaattagaaatctttaattttagtcaaacaatcgagaattttcaaattctctccttttagtcagaaattagacttccttaattttagttaactgaaatttatgcttaaaatttcttgaattttaacatcttccaaataaaatattcaaacaccaaattttatcttaaagaattttaaattcctatttaaattattttccctgttttaaatagttcatccaaacagtgtgcaatggcttttttttaaaaattctgcctcaacaatgaacacctcgtcctttttcaaactaaacaaagttatgaaacctagtagagattagaagaatcataaacaaacagtagatattatcattctaattataaaatctagggttttaaatatcaatagcggtctcatttcggttttattgtgttcactgatatcgcggcaattaaaatgcaacagttcatgattgaggtcatgagcaattaaatttttttaacctttttctaaaacctaacagagaaacaatgtagaaaaccaatgcttcggcgtggatatttgcttgccattgtcccattccatgtaaccaatttcagtaaaaaaaaaaaaaaaactgttgagaacacaaacaagtaagcaaccacacaccaccatttcgaggatttgcaatacaaatttctttctccgcgacagaaacgcaaacgcgaagaaccgaaacaaacacgttcacgctaataaaaacaaatataaaggggaacaaagagtcctccaaaatagaaggcaaagagagagagagagagagagagagagagagttaccggttattgctattctctctctgattttgccacccaaacaccaaagagttgcagaagaaggcttctttttgcaatgaaatgtgcacaaataatgatagtgacgaagctcagaggcccaagaagccactagcaacatggactcctacaccaagcccaacagacaaatcataacaccagccaagttcaaggattatatataaaatattaataaatgtagattattatagttgtgccttatatattgtcctcgaacttttatacaagggagaagaattaatgaagtcattatgttaatattataacaacaatatttgtagatataaaagttatattttgaaattataataaataatttgaattaggctacaaagttccttttaaattgaaatgatataattagttatttttaaacaaattttgacaccaatatattttttgacaccaatttttaaattataacaaacacgcacgagtaacttttagttaatcattaagttttgtttagtgtacatagtagtacactaaagttatattagactttaattggataattgttaaaatataataataataataatttttatgtataaataatttaaaatttaaactttattttttttaatttattatatttttataatcttatttattattttttaaaatataatatataaaattaaattttattttcccataaattagaatatgtataaatatgtaagctttatttttattttatatattatacattatatttttataaatttatgtatttttattttatatattataaattacttaaattctatatatatgatttaataacattgttttctattttttataatatttatgtatgtgatttatggacaataatttttatctatattacaataaataaaatgaatatcttaatatagataaaaaaatattatcactaaaacataaatattatttaaaaaaatatatataatatataaaataaaaataaacattatataaatatacatattctaatgtatgtaaaaatagaatttaatattatatattatatttaaaagataatacataagattataaaaatataataaattaaaaaagaaaaaaattgaactattttcacataaaatttatattaatattatattttataccaaatattcaagcgagcacttacatgtagtgttgaatgagtgtgtatctcactccaaagaccctggttctaatccttcattgaacatttttaaattttgaaatataggaagactaagatagtgaattaaaaaataaggaaaccaatttgatgaacattgggaaccttattgaatttaatataaagcccaaattgggtcgggtttttatgttatagtcaaattgggtctttatcaatgtataaattccacttaaatcatattgaaccttaatgaatcatttgttacaattatactttatattttaaaatacttaattttacaaaatttgattataacatactttcataatataaaaggtatttatttattatcatttttatataattatataaaataaaaaatgagtttgtacaaaacagaaattaaaattataatgataagcatgatttatttatcttaatcaaataattttaaaataggtttttaattttattaaaattatcatagtataattataaatgttatatatttataaacaaatattgtaaatattgatttgtcaatttatgaatattaataatatattcagttcaatatttagtattagattctaaattatttttataaaaattagaattcggtcgctaattcggttgccaatctcaaaatagtattgacttatagccaccgaataacttttggtcgctatcttcataatatagttaccaaatcatattcgatcgctatcttcataatatagttaccgaaacgtattcggttgctatcttcaagtccgcctcggtcgcgaactcggttgctattacacttttgtatgcacagatttatagcgaccgacagtttcagcgacactatctttcggtcgctaaatctgttactgacaatagttagccaccacgcttaagttcgatcgcaaattcagttgtaataagatacataatataattgatcactatttcagtccctaatagccactgaatggtttcggtcgctaaatcccatttttctagtagtgaattacaaaccaagtgacttgacacagatcaattcatgctagacagtgaaaatccctaattggagagaaaaaccatagacaaaaaaaaattcttgtgatccaaaaccattcatcaaagatcgattaagctacaagtatttacagatcacaaagctcagcattcatagataaaatccaacaaagaacctctcttggtgtgcatcatgtgcacgtgtccacgattgagaacatacatcaaacacactttgcaatctaaataaaacaactaatttacacagcaaacaaagtgattattaccaataaaatcaagaataaacatccaaattgtccccaaagaacaacagacataagcagtgtgagttcttcttagttagggcttgcacatatagctaattaatatattattataaagaaattaatatatgcaaatgaatattgcagagataaatctcaaattgaaaattcgattgcgaagggacttactaaataatagttggtgcccaatcgaacacagtcgcagggttgtggttcaatgttcaacatcaggaatatgtgataatatacgtttgttctcttggttatgaacattgggaaccttattgaatttaatataaagcccaaattgggtcgggtctttatgttatagtcaaattgggtctttatcaatgtataaattccacttaaatcatattgaaccttaatgaatcatttgttacaattatgctttatattttaaaatacttaattttacaaaatttggttataacatattttcataatataaatggtatttatttattatcatttttatataattatataaaataaaaaatgagtttgtacaaaacagaaattaaaacactaatgataagcatgatttatttatcttaatcaaataatttaaaaataggtttttaattttattaaaattatcatagtataattataaatgatatatatttataaacaaatattgtaaatattggtttgtcaatttaagaatattaatgatatattcagttcaatatttaatattagattctaaattatttttataaaaatttgaattcggtggctaattcggttgccaatctcaaagtagtattgacttatagccaccgaattacttttgatcgctatcttcataatatagttactgaaccgttttcggtcgctatcttcaagtctgcctcggtcgcgaattcggtagctataacacttttctatgcacagatttgtgcgtcatgtgcacgtgtccacgattgagaacatacatcaaacacagtttgcaatctaaataaaacaactaatttacacagcaaacaaagtgattattaccaataaaatcaagaataaacatccaaattgcccccaaagaacatcagacataagcaatgtgagttcttcttagttagggcttgcatatatagctaattaatatattgttatgaagaaattaatatatgcaaatgaatattgcagagataaatctcaaattgaaaattcgattgcgaagggacttactaaataatagttggtgcccaatcgaacgcagtcgcagggttgtggttcaatgtgtcaacatcaggaatatgcgataatatacgtttgttctcttggtcggcaaacttgcacaaaagatgcacaagttacacgttcccaagggtgcttgtagacgtgatcttgtgtatatgtttttaccattatgctatttcttcactcaattaagtcaacacaccacacaaaagttttgatttagggtttagccgctaataaaggaggaatttcactcaagcaattgaatctgacaggggaaatgaataattaattcatcaagcttgttcaaatcagaagttaatatcaaagaaattaaattaacagttaaagaatcaagtctaggaaaaataagaaacgaattagctacaaaaaataaaattatcgagaggtagatcctaaagaaacgtgactatgcgtcgaaagcaactaccgcataaatcgaattcacagaatcaatcgacaatgcactaataatagagaatcgagggttcaaaaatacaaagatacgataaaatattaaaaggagtgtgaagttaccctaatcgaaggctttgtagtaacgagtgcccacctcttatctgcgctcagattttgcgttttggtcaaagtctaattacgctgttcagcaagatccgaaactttaacctaattaggctggactaagtaatgttttcaaggtgttgctaagtgcaccaagcatattgcttgtgcacctagaatagaaaacaaaattcccaaaatgcccttcatataccacaatgttctaaccaactgagctaataaatcaattatattataaaataaataatgttgttgtatataatagtaaaatttctaatttatatttaatgcacatgtaaatttaaataataagttttgtaacaattaagttgatataacttatgcgattaacattaaatatacattagaaattttactgttatatatagtaatattatttattttataacataattggcctattagcttaattggttagagcatcatgctaataacgcaaaagtcacaagtttgactctttcttgggctattaattttaaattaattcgtaaaacatatttttaaaattaaaaaaaaaacactgagggatcaggtgtaatttgtatgactcatacgaatcacgtgatccgtatgaaggttatttttgggaaattcactttctatgacgggtgcacaagaaatatgcctgatgcacctagcaacacccgtgttttcggtctacaggattggtttttacttccttcatattccattttgcttcttcaccttctagttttaaaatatccctagattaaaaccttccaagtcttgggctttccttcgtacaccaacataattgctagtacaccccataatttatttgaagtgacaaaaatgtatttcatttaaatttttaaaacgcttccttcctcgttgttttctcccttcgtacacttcgtgcctcctcttctttgttctttgtctagtgttctctacgtttgtcatcgttcgtgccgccaatctccgctagcgcaatcgtcatcatgttcttcttcgccaccttaagtgtttattccctctctgtcgattttttgtgctattctagttagtggagactagaaattcatataagactatagattgtcaatttgtataggccatatggattaccaatcgtcattgtttataaggattattaatttgcataattattttttaattataattaattaattaataatttagtcgttaaaaaattatttaataaataaaaaataaatactattgcatgtttttaatatgtaattaattaattaattaattttttaaagttagtttgtaataaatatttaattcaataaattattatatttttgttattttttatgcaaatatattatatttgaaagttgaaatagttatttgtaatatttttatcataaatttatctttactagaatatgggaagtgtagtctacaaatatttttttaaaatgtatattgatttatgaatatataaaagttttcaaaaaagtaaataagttattggtgaatatatttttatgaaagtgaaatattatttgaagaatgacattaatttttatgtctattttaatgtgcaccatagtggattggagtcgatagagaatatttgttcaaataacgtataaataacatgaaacacctttatttcacataacatactttcacaaaccaaatttaatctcaatattatggtatttttgggatagacaaacagtcaaatcatgactaacttgagattagtttttattttttcaaatgaatcaatcattcacaatcaaaatggtgtttattttcatagttctattacaataccaatgggagttcctaatgattattcttttgagacattcaatgttgctctattttttctacaaagtagttatttagaataaaatttataattttttaactacatcattcaaagaattaaaataaactaaaatttttgttgttgttgaatgttacaaaatgtgtataatataatataataaaataaaataaagaagttcattttatcttatcttgaactttacaaaatcaaaattgtctaaattgatatagatccttgtacttgaagttgagttgattgaatactcatctgaaaaataattattaaaatatagttgtttgtaaattaaaattattataataaataaaatataaaaaattgattgtaagcaaaaaatatgtaagactttgaattaaaataatttatttattaaataagttgaattgaaaataattgtcaaaagggatttgtcattccctctattatgagttattgttaacaatggtgttaattactgtattatagtcttatgagtttatctcataactagtttttaattatgtgaatcactttgagaaagtttatctcatttacaattggcctcgatctttagatattttatctctttggcggtcaattagtataattaaatcactaattataacttaagaattatatccaaatatcactactacaaaatacacactttctacatattgatttaaaggcctttttatatcagttattgaactggtgtatagattcaaccatgctacaattttcggaaaggcatcttccccaatcctccccacttacctacatttttttttgttatttttaaattatttgttttcttttcatatctattgacgaattttttcttcctttttggctcttttagataatatgctagactcaattgttttaatatatcctttttttttaacttattgttttgtaaagttgcagttacttggagtttctttattgttgtacggttaattattggagtttgatagtgagaatcaagaattgaaaaattattatacagtgttttctttcattattattatttctaacaatacgtttttctttgaatttcatcagtaaattaccaagttatcttctgctttctatctcatcatcaatattcgcctccaaagtgactgcaaaagacaaaagaaactaagaagaggaaaatggactctccaattatcctggcacaatgattttcctatggtcatgtcaaagcatgtcatgaagattttatatgctttcaattactcgagtggagacaattgaaattttatgtatctagctaaagaaaagtagcatatattgcagaatcataagaattatattaattaagttctcttctccctatatttagacccctccatatcgtacaaagttaaacaatatcaagtctcgttcgttcttacacaagggagtgtcttctgccttcaaacattcaacaatattttttccccaggcagcacctcctcctgccagcagcacaaggttcaaggattttgtctgaggatataaaattggaatggtaactcaatctttgaatttagtgtaatatatgcagatttattccttgtttttgtcacttttgtatttttcctcaatcccattgctcttttcatcttttcccctctcttggatctcccaaattagttatatttattttaaccaaacccagttgacaaccctggcttacttatagtactatttttaagtgtagatatttgattcccttttcggttcaaggtatacaaaaactaccaatttttgaagaaaaatgagaataaaaaaatcccggttttcatattaaactagctatataaaatcaagggcaaagtgtggagacaattataaaaaaggaagcaaattttattagggtagctagtttgattttatcaacaattagggtgatgatttgattttaaattgtaaaaataggtaataaattagggaaggggagtgtatagcgtataatggattgtatagtaccataaagaaagcgtaaaataatggatatttggccatctaacacacacattaattgatcacttctcttttgataaaatttattactattatcatcattattttacaatatagtgataacaaacatggtgaatgcgtcacagaaattaatggtggtgtgagagagaaaaagaaaaaaggaggtagctatagctaggaggaagatcaggagcaaaaaagaagtactactgcaagtgactactactatggcatcttcaaattcaccatgtgcagcatgcaagttcttgcgtcgcaaatgccaacctgagtgtgcatttgcaccatattttccccctgaccagcctcaaaagtttgcaaatgttcatagaatttttggggcaagcaatgtcacaaagcttctcaacgacttgcaccctcaccaacgtgaagatgctgtcaattcccttgcttatgaagctgagatgaggctccgtgaccctgtctatggttgtgtaggagtcatatcactcttacaacaccaacttcgccagcttcaaatggacctctattgtgcgaaatccgagctctcgaggtaccaaaacttgagcatcaccaccaatttgaactctctagcttccgaatccgcgaccacggcctatcatcaccatcaccagaacatgtctggtgctggtgccggtgccggtgccggccgtgaccatcattatcactatcatcgccatcatcagttctttccaagggaccaacaccaacaacaacaagtggtgagaagctttgatgccggcaacagctatgatgcaagtcttttggccatgaacatatccgccaacttagggcaactcaaccagcttcagcatcatagtgcagctggcgggggcgacaaccgcaactgctcctagggtttcatgggggtgacatatttaatgcaataggtaattaatttagttaaacttttttttcttctctcttactcttatttcagctataatggttcatttaatttgtggtttgttaattatcatcattactgatatccacataaatgcatattttgtcactcacaatgatgtataaacatggactcataacgttgccagattcttcaattctatacacatatatataatgtaagcagttactcaagtgcttgccttagttaatttggaaatattactctacaatgtgctgtctaggtttcgaaatgttaatctcactatcaattgtcattaatctggtgcatgagttttagcacacaattgttttaataatatatacttacacacataaatagcatattggtgagacataatgcatgcgttaaaaaaatcgtcataaatgtataatcaagatgtatgattaaagattaaatgaacaacgactaattttatgtatggttgaatgattaaaattaggtttttttttttgttaatgctaaagagttttgacagtacaatggagaaaaaaatagtaccccccaatcttcatttttgacgggtacttctcaagactaagataccgtggaagttcgagttgcattttaataatttttttattcattgcaaagcatctaggcagaaatatgacgtcaataaaaaataagaagaggattcctccacggaaattaagatttgattattaattagcgaacagtttgttctcgtcttttaatcagtaatgatagtacagttttggtcaagtttacgaaatttatggtccaatagcttggcatggtgttagtactgtttcgtaagaatatttgtttattgttctccgtaaccagacgtttaattagagtaagctactattttatctacgaaagtatatagggcctgtttgaagaattttttagagtttctctgctaaaaattagagtcttttttttttttttaaaatagacaaattttcaaaacctttgaaaagttcttctagttttatatccaaaaattttcatgtgctataattatgaaaattttcaattgttttatttaagttttttttacaacaattatttcattcaagtctttaaacttaatcactacttaaacatacatttttttgttttttttatgagttttcgaaacatatattagtattattacttaaatcacatatatactttttttatataaaatttagacaaaaatgtttggccaattacattttcataaacttatttaaaaatttcttaattttaagaactgatgtgataatactgcatgtacactatcaagaactagtacactgggaagtactgtgtatatatattagtcttataattaattgtatcgatttttttggtcatgcgatactttgctttgccaatacacattaaacagttgaagtacttagatacgagaaaaaaaaattagagatgattagactatgctgtaaaagagaagtgcatttatagcagttgcttctgagtaaaactactcaatcaaccctacgtttaattctgaatgtcattggcatttattagacagtgccaacactatggacctgttcttctctatcagaacgcctttgttaattccattgggtatgaaatgaattcaatgactgtgacttgacccattcttttaccttttttccttctgccttatcacactgctatgctatcccctcttgactattccttgtttctatcaatacacctttttcttgaagtttggcattgcacgcttttctatatttgtaactacatacatattcatctttcttccaagttcaaactgttaaataaaagaatatatacacatcacaaaaactatgcccaataaaccacctactgcatttctttcatatctttattacttccttaattacatgctgtttgtcccctcccatttctccttttccttctccttctctttctctatatattgtctcatcccggagaaaagaaaaaggaaacaaattaattaataacacactttgattgtccatgagatgtgacaaatcgacaacgtaatgaattgcattttgcaaatgcatggacactactcaatctcaagtctaacagtaattggagtagttgatagtatttgggttttatatttcatacaaagattttatttatttatttaacagttgtatacccaatttctcatttgatgacacttctggtttactattgacattttcagataattttaaatatataacaatattaactcagcattgtaacaaattaaagaacattttgtaacagttctatatttgctatttgaatttaaattatgataattttttaaaaggaacataaggcaaaactagccaattcaattaatttgtataaaggaatatgacctaacgtaacgatcgattctttcatcacgatatacgtaataattaactttgtaatcagggatttgtttttataaagacattgacatactttgttctacattttccccattattaatataattttatatatgtatgttttatgtttttttagtaaattaaggtatagatatccataatttttcacttaataatttctttcttgtagtatatggcaatttcgtaacaattatgttaaacaggtttggtttgcaggataacaagctaacccgtagaccgtttaatgttagcctaaatattaaaatactaaaacaataccaaatgtttaatgtgaggtaggatttataagtagtactacttttaaaatatatctaccccttaatattttttagttaatatttttagtaataattaaatatattaaaatgatactttaaaatagtgagatcatactatttacttaatgtttagtattaaataaattaaatattaagtattttctattagtcttttagtattttaaaatattttaatattttaaatattatattaaaatgattataaaaaatattttgaagaatgtaaattacttttatttttattagtataccttaacattgtatccccaataatgtataagctagcagcatatagaattataaaaaaaaattgtgagaaaactgtgctcgcacctgataatataatgtgatttattgattttaataaataaagaaatgataaataatagaatatactataagtcatcagtcattatggacagtagattggcccgttagcccgagctatctgagggctaatgtgaggctaaaacaataaggtagttgctatttgcactcttatattttacttgtacaattccttttgctccattttccccgaaatatcctcttaaacacactctccctgttccccctccgtgctactaaaagcaatgcactcacccactgcttgagcatgttgggctgctcaaaataattaatttttaagaaattataataactggctagttataaaattataacccatttttatagttatggtcaattcaaataacatatttttatttaaaaaataattataattatttacataaaaatgattatttcaaataatttgtttttattcttcataaccaattatctaattatttttagctgtaacttattatataactagtttatttataactgattatatataatcatattttaaaacaataactgctatatcaaattataactaattatataaaaaatagttatgattataattataattatgtaaatactcacgtcaagaatactccttataaacaagaaccttggaatcaggtgtgatggcaggtctccaattgttggaattcgtaactattgttgaaagggctatcactgtgccaaaagagatgcaaagtgttgaatgtttataatattattcgcctccttatgggttaaatctagacgaaacattctaaaaggaattattttttaaattaaaaaatcagagtaaattttaaataaataattaaaatttataaataaatacttagaaaggaataaatcataagatatgtaacgacttttgagttcataattgtatttttttttcttttcttataaaaaaattatgattttttaatttttttaaatgtgactttgaagtcacaaattatttacggcttttaaatcataaataattttatttaaatttataatttattttaaattttatttaatatttttatatattattttacttaatattcattattttttatattgtttttatttaatatttttatattattttatttaataaatttggtttttttatattttatttaatattttctatattaaataaatccataacattattcacaactttaaatcgtaaataattttatttaaattaatatttttatatttttgtatattatttttatttaatatttttctatattttttatttaatatttagtttattttttatattttatttaatatttttattttataattttaaataatatttttatattattttatttaatattttatattttattaaatattttaatattaaataaattctttacattagaaaaaaataaaaaaaatattaaatagagaaataaaaaaatattaaataaaactaaaaatattaatttaaataaaattatgactttaaagtcttaaataacttacaactttaaagtcaaatttaaaaaaattaagaaattataaaattttgtatggctacattagaaaaaatagtcataaaatgatttacaattttgaatttagaagttatgagatattttacgaattatttttgttgatatttatttaaaatttatcgggattttataatttagaaaaaaattaagaaatgtttggccttaaatctaatcatatctggggttttctttatccgcttgtcaaagagatacaaagtgttgaattattgtttataatattattattcgttttatataagctcagtgttgagaaattcacggttttcatgttcaaatcttattattatcagatatactatacagtatacaacgtgaaattctattttatatttattgaaagaaatattagctgaacttcaagaattaatatgaatatataggatcaaatctatgtgtaccatgcctaaggaaataccgctacaggattggagtgaatatatattccaaaaatctaagtctatgttttatagaaaatggtcaaatatattcagcccatgtcagactcgctttaattaatcaagtgggaggatatgataattaatgtattgaagttattaaaaggtgggagtattattcaaatttaaaataaaagtaatttttgatagtttctttcaaacaaatttcaaattcatcagaattttttttttatgtgaatcgtaggattaaaaaaaaacaatacactcactttaatttaatatactttaaaatgccaggtttcctcgtcacacatgaatgaccctggtacgaataaaaatgaaagaatcaattaaaataaaaagaaatagcagacagaagaatgaataatatgccaaaaaataggaaatggaagaaatgctaagtgggactggagggggaaaaaggatgccaagctgtgtgttaattaatagttggtccaattcttattttataaagctactttatttaaagttaaaataagaaatttgatgcctagtgctagttctctgttgtttattctaagacaaggcaataaagcagcccactctttaattagctttttcatattttcataaatgcttgattgagtagatccttcatcaggtggattaaatcatcttcaatgatttggagatgtaatttctgcttcttctttcagtcaataatattcagcattttaccctaaaaaaattaaaataaaaaacaaaaaaatatttgaccaagagatgcccagattccttattcttttaacaatcaacatcattttttcttacaaatttctcaattttaattgtattgaccatcttcttaatctaatttttgaagatgtaacacaaatatacaataaacaaaatgtttggtttctaatgacgtaaacctaccatgaaataaatgcataacttattttaaatcgaaactctaaatattaatttgtgggtttaagattgagaagaacaaaacagctagagaaatagatgatgaaaggaaaacagagatataacacaaactacagaaactaaagttattgcattaaacataattaaaaatacaaagaagtttcgataaaagctctctctaagtctaaaatctattagaataaatattgacaaatatctgatacaacttaatatttaatttaccttttcaaagtttaattgatatttagtttcataaattcaaacttagacttatacaaaaccaaaattaaattggaagtaacaattgcaattgacaagaagaacaaagataagataaacaaaacaaaaagaatatataaataataataaaaaaaagagaataattattaatatgtctcaaaattaaagttattaatttattatgtagttataaataacatttaattaattcctgaaataaattataatcaaaccaatattacagaaacgaaaacaaattcatttgattacaaatataaatacttacaacctttaaaataaaaatagtatattataaaatgaaaagatattaacaaataaaataaataacatctaatattttgtgaatgtaattatttcaatgaaaactataaaaaaagagtatttatgaaaaaggagtagtatatttgtttaaaaagaggaaaacatattattatttttgtaatgaatactataaaatgggaataaatattttatcaaaaatagaataaaatagataattaatacttaaatattataatttttataatcaacatctttaaataaaaaaaattagtttaataataaaatattacattaaatatcaagggaccttggccccctccaatatgagtaggtccctccctgcctatgttgtgtccatccacctttatcgtccaacaactttattacatatcatgtaaacattatttaggattatagatcaaattatttagaccatccaataatttctcttatacaagggacgcctcaatggaagcaaattctccgtttatgattctttttacttgtattgtagtcgtgtacttctaagctattaagaagagtagagaagaacctcacatggatcatgcatgcgtctaatataacttaaaaattcacaaattaaccagattcttgtttatttaattatttttaaacttgaatcttcttaatcttatagattacaaattatatcaaagggcatcactcatgatagcagaagcactcagagtatattattttctttgttcgcaattttactttatccttttggaaacataacactctttatagtaccgtttgccacccatattctttccttgaaggtttgaatcgaagtaggtttatgagtagcaatagtttagggaatcctaacgaatatattgtcggttatatccttaattattagatagctatctatcatacatcatgaacccattaagggcatattatgaaatctatacgagagagcaaacttaggggccacaaaagctacataaattttatattatgtaattcactctctttcttgatccatactgaagctcgttattttggctttgcattttaaactagtgtacttttcttggctctataaataggcatttttgggtttcaattatttgcctaagtactttggaagattggtagggtgaatcaccaacatgtctaagcaagtttccagtagcactatgccacaggagaagctcctgctgaacccatttgaagtcagtgacgatcagatcttggagagaatatacataacccatttccattgtgttgagaagtatgacgttggagttctttatagtgttgcttcaaatgttatcaaacactcaattgaaattgctgacatgattatcaaggtaaccttgccaataaactttacactcttgtatgtactgttgttgtacattgaacacattaacattttatttgaatgcaaataaatgtcggataatgatgctagcttgtccaaatcacttagtgataccaatcttaacttatctagtcaattggaatggtccaatgtgttatctatgagttacatgtacaattttggaagaagaaaaaaaataggctaaaaagtcaatgcatctggatcttctgccattgttgagtaagagaaagacacattatatcatacaaaaattcaccataattgattgtagtgtatcctgtatggaagagaatctatatcctgggttcactggaaatacattttttaatgatttaggtcacacatgtatctatatactcaattaatcaatgatcattatatgtgacttgtacatgattgcaagctctttttagttaccaattaccatgtatgtgtttataattttctttacaggatggccaacaaattgaacaagtcagagaagaaacggatccattaacctctttccaacgactccctgccatgaagcgaattgcttgccaggtaaagttagaattaaattaaaaaaaatcttttaaaattttgagaagttatttaactatgagttaactctgatttctatatttctgttaaaaaactcacataaatataagaaattgtttgatggtctggaatcatttgatatcttgatatgacacgttattaactctttggtaaattgaaaaatgtgattacttatcacatgaagattgggtggaccattttaatcgagaccatataagaatttccctagacacgcactcctatgataaataataaatgttttgcatcaatatatggtaacaattctcaataatagcactacagtttagagaagaagaaataattatatatttaagactcaaacttgagaacattggttaaactgtaaaaactgaattggttgcagatgatgtgcactgctcgcggcgagcaatatgcgcatcaaacaacaatgttgatacttgaacagctgagagattattcctgggatgcaaaagcagtgatagtactagcagcatttgctctggagtttggaaagttttggcaacttgcgcatatcccacgtgacaaacttggacaatcattggcagagttgaatggacttcaaagcatcatggaaaacatacagcacctggctaactttaacaatttggtgaagaagattgtgcaagtggttaaatgcatcactgattggaagaagatgattactgcggagtataacgtaaaggatgtgccttctttgacggacactttgcacgagattcctgttcttgcatactggacaatcagcacattggtcacttgtaccagccacattgatttcttgggtgataagtaagcttaattgattcatacccttgcataaaatttgaatctcgtgttacctatgcctatgaagcacggacacttcacttatttgtggtgtcagatatgttttcaacaccgacccacctaaacacttttcagtacttcttatgaggtgtttaatttaaaaattatttgtgttgagttaaaacacttaagttgtcaccttacataactcatacagttgaaaaacatagaatgttggtttaaaaagatgatataccaataatttagatatttttcttatatgttacttatattttatttcattaaagtctttatgtttgtatgtgtatacggtgttcccgtattttaaattttagacattagtcatgtcaaaatatccgtattattctggtgtcaatgtcgaagttcgtgctacagttagtaagtagtataattatgtgatgctataagagagacaacaacaacaaaaatcttatccagctagataaatcatatgttatttggctcgattaaagacaaatttttagagttatcttgaaattgctttaacaatttcttttagtttccttttggtcactctccttttcataagattaatatccatgtaatttaatttactttcctggttggtgcttccaatagtcttctttgtatgtgtccaaaccttcttaactaattttctcaatcaatgtcatatcaatatcttcttgtatgcaaccatcaatgatataagagagaaagacacaaaataatggtgttaactacttcatattgtttttgtgtctctattgtaatatgtcattatcatgtcacttattaacatgagaagttccaaatatgttatctatataataaaatttgtataatctataactattataaagggaataatcccattcggtgtcatctttttttttgaacatttttatccttaagtattataaagtactataacattttattttaactttcaacgacaattatgtctttcaaaacaagttacaacccaactacaggtaacttcaaagtccaggtacagagtgtcatttttgcactagtaattcatagttgcaattgcttggattcaggggttacagatacgatttatccaaatttgactataagcttgacttcattctcaaaaatttcaaggaccaccaggacaagtgcagcacacagataggttagttgcatgttttatgctttgcagatcgattgtatgaatattagaagtgtgcttcccgtaaattctctgaactagtgaaggtgcagaattgaactaaaatggtgataggaggttaaggttctctgtgttaattgcagggcaaatagaagattactcgaggcgcaaggatattatcacttccattcaaactgatacacaaattgatattgtaaaatttttggaagctcttattatccccagttattcccaggattccagaccaatagtgtataatggtcttactggaccacaggtatcaactatagtaatagtactatcttaataagtgtcctatctctactacaactatttccttatgctaagaattaatccaatgcacattgaccataaaacaaagggtttaatcttttaaatgtcctagtctttagaaaaataacatattcattgttcattattatctccgtgactcttttgcaggttgcattaggagagtttaagaataagcatgtcttattgttcatttctggccttgaccacattgacaatgagattcagcttttaaaatcgatcaatgcgaaattgaaggaagaaccaaacgagctggagggttataggaaagaggatttcaagattttgtggatccccatagtgagtgtttgggatgaagagcaaaagaaaaagttagatgttactaaggttgagtggtatgtggtgaaggaattcaatttccaaacaggtattgatctgataaaagaggtattcaattacaaaggtaaccccattatcatgctcataagccctgagggtaaagtggaaaactctgatgcaaagcagattatttccaaatgggggattgatggcttccctttcagaacctcagatcacactcgactcactcagcaatggaactggttttggaatgaaatgatcaccctcagtcctataataagagaattggtaagtaccaagcactacgaagtacaaactaatgattgtcctgcttgttatttacataattcttgggtgattgttttttcattgttggatttctccgaagagagtgccacagtagtacttgaaccagtgaaaagcatcaaaataagtagtgatttgtgttaatctgttacaaaatgaaatcatgttatcatgcatattatctttatgtttaattaaaagttaaatttctatgcactattatggaatcaaaaatcatgttaggtatatttttaaaataattattataaaaattaacaaacttaccttataatttttaattggataattgtataaaaactttttatgctatcagaccatctaaattaaattttttaaataaatacaacttgtatatattgatgaaattatctttaaagggtgttgcaacccaaagtttttttggcattatctttatcttataaattgacatagaacttaaaagaaattgatctatagttttcatgtccacaaattagatcaaaagggactcttacatcttcatatatggaggcaccaacactaagtggatccaagatttcaccacagctgtggagaaattgaaaaagaatgaaactcttaccctagaggaagaaaccacaattgagtcctatccattgggaagggacagcccaaagattgttccgcgcttctggatcaccattgacaacttgcttgctagcaggaaactaacaaaaaagggtagtgaacaagtgcaagattctacaaccagagagattcagaaattgatgttcctcaaacaagatccacttggatgggccattcttacaaaagggtctcatgtgaagctcctaggtcatggtgatgctatgcttagaactgtcacagactttgagagttggaaagggacaatgcacaatgaagtgagctttgatgtagctttcaagaactactatgataaatgcaaggttaagagtgttcctcccaaatgtgaacatagggaatttgccaattaccccactgacattctagctcacataccatgtccaaataagtgtggacatgaaatggaagtttcttctgttaaatacatgtgttgtcatggtcgtgaagccagtgacattgcctaagacaaatgcatgaaataactgctacatgctcagtggtttcttcatttcatgaatgagacttcttcttcaagtttggtttaaatttttgtgtgtagatggggcaatatatttttagtatatattgattcttatgcagtttgccgtagatgagtattatattgagtgaattgtttagaaagaataactagttctgtgctctgcttgattggcgcagaatcatattcaatcccttcctgctccccaagcttttggttcagttaaatatacagtcaactatttggctcatgcactcatcattgtaacgaaatttaagtattgttgaaacaaaaagttcaaattgaccaataaagtcatgtttaatagaagtttaggctgtgactttattattattattttaaatagataaattatatattttaacttttaaggcgttaggcaaaatttatatttatatttaaaaatcaacaaaataataattacaagcattattaattattgataatattttaaaaataatgaatgtatttaagaaaaaaaattaaataattaatatttttagcaaattatcaatttttttgtccatacttcttgaaatatctttatccaaaaaaaatttaaataattaatatttttagtataaaacctttttaagattaatataatatttttttaatccatcttcatccaaaacaattgtttatttaatagaacttaagtctttctaaaaccaactcgattaataaaatttgatcataaaccaagcatcgcaaacaagcaaacacaacacgaaaccaatgaagatgcaagcataggcaagaataacacagttcattagttccacacaacaaatcagccatcactaatcaaccctaggaagaaaccaaagagagagagagaattcgaggttgtgagagacgaaccagagggtgcaaggatgcgagtccaagaatttcattattttagtggtttaaaattctaaattttagatttctacttaataataataacacatccaaacagaagtttattaacgctttatttttttggggtgttaaagaaaattttgaaagtagtttataattttaattttttaaaattatttttttctaaataataaagaagttactgtcatggaaaaggaaaaagattctagaagatatgtttacattcaagaataagaagttcgttccaggtttgattttattcatcttttaaggagcattgataagaagtatgaaagaagcatgctacaaagatctaaaggtacgagaaagagatatttatagtaaaaataaaatattgatatatttgaactaaaacaagcgtttgtttaatcttttttttctaaagaaaagtgattttatagtaaaatatttttttttttgctttttttaagcgagtatcttttttccttataaaaacactttttttaattgcttctttttaaagtttaaacaaaatgaacattaatatcaaattaatgtttattaaaattttaaagattaagaaatatttcttttaactgtcttttaaaattctcaattattatgatttgagagcttggagggatgcaagacaatgatctcattggacttgtcatgataaaaactttactcaattgatcaattgacaacacacattgagtttgagtttgtaaaaagatgtgattttgattcccacataataatcttttggaaatagtgataaggtttaaatattattaagcaaaattaatctcataattgatctaaaaaatttaaagttataaggataccactagagagcttaacacatgatactaaatcaaataaaattgtattaagctacacaagcttctttcaattcatgttaccacaaaagaaactcaaaaagttgatatgaattatactattctcaacaaattttgttgcaatacaagttaagatcagatgcaacagggtctacttagatcttgtagccaaggcaacaagaaagtctctgtagtagtcaccagatggaaagccttcacatatagcatcagcaagttccttcccatacttttctttgaaaacccttctaatttcatctatgtctatctcagccctgcttaccaatgttcgtgccaaaactcttctgtcccttgtttcccctttgatgcttgagtacagtatcttccattattaatcaaattgtaacttttaaaacaatgatttcattccaaatcacctaaaaaataaaatctcttcctaaactatatttactgttttaagatttgtaacaagaagttaggaacaaacaactgcaccctaccctatgccagttggttctgtgaaggaaaaaaaaagaagctaaaatgattacctttgcataatagtgtgctggattacaaatgcatttcacaaccaccattaaagctttaccaaattggccatatttccccctcttgatagactgaaacacagtccatagaatgacacacaataaaattcaaaatccttcaaataatgacaacgcaaaaaaaagggtaagatggggaaaagactagtagcatattctttttattgatatacctttgtgtagtcatgcccataaatatgtttataactaaaaaatgttagctttagttggggaatgctcctcttgcttaaaatttccaacacaacagcttcaattacagttcctaagcttccctccccagtctcatagagcctcctagcatcacacttggatatatgatggttgacatctgcttgatgagctttgtgtgatgcagccaatgccacaataatctgcaaaataaaaaatcctcttctttaaataaatttaccaaaataattgacaattcccagaaaccaattgagtgattcccaagaatatatctctcaccttttgaaaggggtgtggagggtccaaattgataatatcttggtccaagtgtcttttgaacattttctggtaggcctgtgtgatgagaagaacatgacttgattttctacccacgaaaatctccacaagagccttgaaattggtctcatcttgttggagagcttctctagccacaacagcatccctgccatgtgtgtcaagcatccacaaggaactagcagaacagttcatagaagaagaagaagaagaagaagaagaagaagaagaagaagaagaagaagaagaagaaggagaataaaacgcatcttcatatctttgaagatgacttatcaagtcttctccattcactgccttaaaggtctccctgagttgttgtctttcatggtgggttacacaagccagagatgtgatcaattgactcaaactccccaaagaatcatgagttctcttgcaatcaagttcgaaattgaggtttgtcatggtaagaatgttgttagccatatgaaggaaacaaatggaattaatggttttgttttggttcaggttcaagtaaatatatagtccaccaaccatatgtatttaagcaacaattacttggagggtaaggcagggatattcctactttttagtttttgtgtattccatggaaagacaaaaggcaaaggatgaactcatcatcactcactaattgctacatatagggttggcttcaaagctcaaaaagtctttagagcaactgaagtttctatgcattgagttgatggtgatgtgtaacgcaggttgtacttgtatgtgcatgccacgtgaaagtttgttttgtagctaaatttttcattggtgtccaccatagcacattagcacctttccaatcaattttttttaattaacatcaactaacttgagagttgccaatgaataacaatttattattcaatcagaacaaccactctttaagacttaagtaatgcaactttttatggggttatttattgtgtgtctatggacacataaaattattaatacttacatcgtcttagacttttgtgttttatttacatagtaaatgcattttttctagtataaatattgtttttcaacttctcatctatttacatattcataaaagaaaatctctattagttaaagccctttttaatttagaagtagaaaaatattctaaatgagataatcatatttaagtcaaaagtaactacattatgatgaaattaattatcataaaatatatttttcttatatacatgttcatgatcaacttaaaggataaaacaactaaaatttatgttttagacacatcaaataaatatttgctattagtatttataaaataaatatagaccttatatattaataaaaaaaagttacatatgttagcaaaaagattcaacatatttaaagtaaaaaaataaatttattttaaatttcacttatggttggggaggttttgtataagactcaaaatcacggaagggaaataaagtatatttcacctcaaccaattaccggttaatattaatccattctttctaatgttaatacttgttctgaaaaaaattatctatttggaaagacaattaaattgacttaaaaaagctagaaaagtgacatgtttagtaaaggaaaagaagtaaaaatattgatctgatttattaacaaccatacaacaacataaatagaaaataatggttgatgactcatttacacacaaagccacaaaggtgctgttcttaaaggaaatgatggatcatgttggttcaaaatgaatccgcagtgcctaccccactaattccacagggagtacctaatctttgggctagcttatggttctgggtttttctgttgtttaccttggtataggtgcatggaaggctgataatacagacatgtaccataaggttattattttagatacagggtgcgtaacccaagatcttgaatttcgatgatggcgtcctcatcctcttccttgtcaccgtctcaggcatggcaaccgtgatggtttccttcacgcattaagctcgacg